(19) United States
(12) Patent Application Publication
(10) Pub. No.: US 2010/0311749 A1
(43) Pub. Date: Dec. 9, 2010

(54) 4-BENZOYL-1-SUBSTITUTED-PIPERAZIN-2-ONE DERIVATIVES AS P2X7 MODULATORS

(76) Inventors: Laura Jane Chambers, Essex (GB); Katharine Laura Collis, Essex (GB); David Kenneth Dean, Essex (GB); Jorge Munoz-Muriedas, Hertfordshire (GB); Jon Graham Anthony Steadman, Essex (GB); Daryl Simon Walter, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/739,462

(22) PCT Filed: Oct. 24, 2008
(86) PCT No.: PCT/EP2008/064429

§ 371 Date: Apr. 23, 2010

(30) Foreign Application Priority Data
Oct. 26, 2007 (GB) 0721096.6
Oct. 15, 2008 (GB) 0818905.2

(51) Int. Cl. A61K 31/5377; C07D 241/08; A61K 31/495; C07D 413/10; C07D 401/06; A61K 31/496; C07D 405/06; C07D 403/10; A61P 29/00; A61P 25/28; A61P 19/02

(52) U.S. Cl. 514/235.8; 544/383; 514/255.02; 544/121; 544/360; 514/253.01; 544/374; 514/254.1; 544/372; 514/254.01

(57) ABSTRACT

The invention provides a compound of formula (I) or a pharmaceutically acceptable salt thereof:

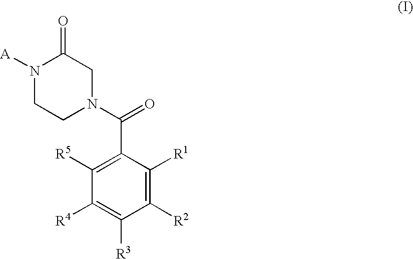

wherein:
A is C₁₋₆alkyl, C₃₋₆cycloalkyl, —CH₂—R⁶, —CHMe-R⁷, —CMe₂-R⁷, or optionally substituted aryl; wherein, when A is optionally substituted aryl, said aryl group is optionally substituted with 1 to 3 substituents, which may be the same or different, selected from the group consisting of halogen, C₁₋₆alkyl, —CF₃, C₁₋₄alkoxy, C₁fluoroalkoxy, cyano, NR⁸R⁹, and pyridyl wherein the pyridyl is optionally substituted by one methyl;
R¹ is chlorine, fluorine, —CF₃, cyano or C₁₋₆alkyl;
R², R³ and R⁵ independently are hydrogen, fluorine, chlorine, —CF₃, cyano or C₁₋₆alkyl, such that at least one of R², R³ and R⁵ is other than hydrogen;
R⁴ is hydrogen.

These compounds and salts are thought to be P2X7 receptor antagonists. The invention also provides for the treatment of pain, inflammation, rheumatoid arthritis, osteoarthritis, or a neurodegenerative disease.

The present invention relates to piperazinone derivatives which modulate P2X7 receptor function and are capable of antagonizing the effects of ATP at the P2X7 receptor (P2X7 receptor antagonists); to processes for their preparation; to pharmaceutical compositions containing them; and to the use of such compounds in therapy.

The P2X7 receptor is a ligand-gated ion-channel which is expressed in cells of the hematopoietic lineage, e.g. macrophages, microglia, mast cells, and lymphocytes (T and B) (see, for example, Collo, et al. Neuropharmacology, Vol. 36, pp 1277-1283 (1997)), and is activated by extracellular nucleotides, particularly adenosine triphosphate (ATP). Activation of P2X7 receptors has been implicated in giant cell formation, degranulation, cytolytic cell death, CD62L shedding, regulation of cell proliferation, and release of proinflammatory cytokines such as interleukin 1 beta (IL-1β) (e.g. Ferrari, et al., J. Immunol., Vol. 176, pp 3877-3883 (2006)), interleukin 18 (IL-18), and tumour necrosis factor alpha (TNFα) (e.g. Hide, et al. Journal of Neurochemistry, Vol. 75, pp 965-972 (2000)). P2X7 receptors are also located on antigen presenting cells, keratinocytes, parotid cells, hepatocytes, erythrocytes, erythroleukaemic cells, monocytes, fibroblasts, bone marrow cells, neurones, and renal mesangial cells. Furthermore, the P2X7 receptor is expressed by presynaptic terminals in the central and peripheral nervous systems and has been shown to mediate glutamate release in glial cells (Anderson, C. et al. Drug. Dev. Res., Vol. 50, page 92 (2000)).

The localisation of the P2X7 receptor to key cells of the immune system, coupled with its ability to release important inflammatory mediators from these cells suggests a potential role of P2X7 receptor antagonists in the treatment of a wide range of diseases including pain and neurodegenerative disorders. Recent preclinical in vivo studies have directly implicated the P2X7 receptor in both inflammatory and neuropathic pain (Dell'Antonio et al., Neurosci. Lett., Vol. 327, pp 87-90 (2002), Chessell, I. P., et al., Pain, Vol. 114, pp 386-396 (2005), Honore et al., J. Pharmacol. Exp. Ther., Vol. 319, p 1376-1385 (2006)) while there is in vitro evidence that P2X7 receptors mediate microglial cell induced death of cortical neurons (Skaper, S. D., et al., Glia, Vol. 54, p 234-242 (2006)). In addition, up-regulation of the P2X7 receptor has been observed around β-amyloid plaques in a transgenic mouse model of Alzheimer's disease (Parvathenani, L. et al., J. Biol. Chem., Vol. 278(15), pp 13309-13317 (2003)).

JP49110680 (Tanabe Seiyaku Co. Ltd) describes a series of 4-acyl-2-piperazinone derivatives for a range of disorders. US 2003/186960 (Lauffer, D.) describes a series of cyclised amino acid derivatives which are claimed to be useful for treating neuronal diseases. WO 95/25443 (Merck & Co Ltd) describes a series of piperazine derivatives which are claimed to be oxytocin or vasopressin antagonists. WO 2004/101529 (Ono Pharm Co Ltd) describes a series of nitrogen containing heterocyclic derivatives which are claimed to be useful as p38 mitogen activated protein kinase inhibitors. WO 99/37304 (Rhone-Poulenc Rorer Pharm Inc) describes a series of heterocyclic compounds which are claimed to be useful for treating unstable angina, stroke, etc. WO 2003/017939 (University of Yale) describes a series of piperazinone compounds which are claimed to be useful as GGTase inhibitors. WO 2006/034315 and WO 2006/034440 (both Xenon Pharm Inc) describe a series of heterocyclic derivatives which are claimed to be stearoyl coenzyme A desaturase inhibitors.

The present invention provides compounds which modulate P2X7 receptor function and are capable of antagonizing the effects of ATP at the P2X7 receptor (“P2X7 receptor antagonists”).

In a first aspect of the invention, there is provided a compound of formula (I) or a pharmaceutically acceptable salt thereof

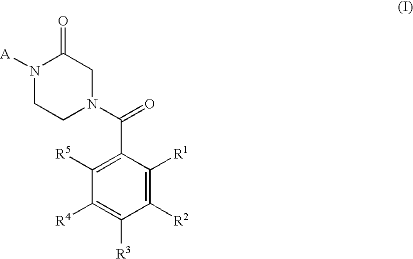

wherein:
A is C₁₋₆alkyl, C₃₋₆cycloalkyl, —CH₂—R⁶, —CHMe-R⁷, —CMe₂-R⁷, or optionally substituted aryl;
wherein, when A is optionally substituted aryl, said aryl group is optionally substituted with 1 to 3 (e.g. 1 or 2) substituents, which may be the same or different, selected from the group consisting of halogen, C₁₋₆alkyl, —CF₃, C₁₋₄alkoxy, C₁fluoroalkoxy, cyano, NR⁸R⁹, and pyridyl wherein the pyridyl is optionally substituted by one methyl;
and wherein:
R¹ is chlorine, fluorine, —CF₃, cyano or C₁₋₆alkyl;
R², R³ and R⁵ independently are hydrogen, fluorine, chlorine, —CF₃, cyano or C₁₋₆alkyl, such that at least one of R², R³ and R⁵ is other than hydrogen;
R⁴ is hydrogen; and
R⁶ and R⁷ independently are C₃₋₆cycloalkyl, C₁fluoroalkyl (e.g. —CF₃), —(CH₂)ₘ—O—C₁₋₃alkyl wherein m is 1 or 2, —(CH₂)ₙ—CN wherein n is 0 or 1, tetrahydrofuranyl (e.g. tetrahydrofuran-2-yl or tetrahydrofuran-3-yl), tetrahydro-2H-pyranyl (e.g. tetrahydro-2H-pyran-2-yl, tetrahydro-2H-pyran-3-yl or tetrahydro-2H-pyran-4-yl), unsubstituted pyridyl, or optionally substituted phenyl;
wherein, in R⁶ and R⁷, independently, the phenyl is optionally substituted with 1 to 3 substituents which may be the same or different and which are selected from the group consisting of halogen (e.g. chlorine or fluorine), C₁₋₆alkyl (e.g. methyl), —CF₃, C₁₋₄alkoxy (e.g. methoxy), C₁fluoroalkoxy (e.g. —OCF₃, —OCHF₂, or —OCH₂F), cyano, NR⁸R⁹, and pyridyl wherein the pyridyl is optionally substituted by one methyl;
and wherein:
R⁸ and R⁹ are taken together and are: —(CH₂)₂—X—(CH₂)₂—, —(CH₂)₂—X—(CH₂)₃—, —(CH₂)ₚ¹—, —C(O)—(CH₂)ₚ²—, or —(CH₂)ₚ³—CH(R¹⁰)—(CH₂)ₚ⁴—;

### X is O or S;

p¹ is 3, 4, 5 or 6 (e.g. 4 or 5);
p² is 2, 3, 4 or 5 (e.g. 3 or 4);
p³ is 1 or 2, and p⁴ is 1, 2 or 3 (e.g. 2 or 3), provided that p³+p⁴ is 2, 3 or 4 (e.g. 3 or 4); and
R¹⁰ is OH or C₁₋₃alkoxy (e.g. OH or methoxy, such as OH).

In a particular embodiment, the invention provides a compound of formula (I) or a pharmaceutically acceptable salt thereof

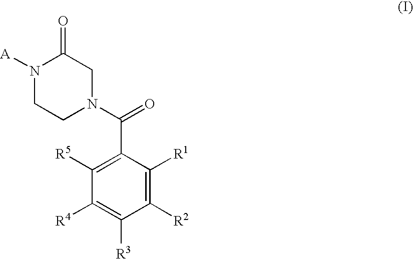

wherein:
A is C₁₋₆alkyl or optionally substituted aryl; wherein said aryl group is optionally substituted with 1 to 3 (e.g. 1 or 2) substituents, which may be the same or different, selected from the group consisting of halogen, C₁₋₆alkyl, —CF₃ and cyano;
R¹ is chlorine, —CF₃, cyano or C₁₋₆alkyl;
R², R³ and R⁵ independently are hydrogen, fluorine, chlorine, —CF₃, cyano or C₁₋₆alkyl, such that at least one of R², R³ and R⁵ is other than hydrogen; and
R⁴ is hydrogen.

As used herein, the term “alkyl” (when used as a group, or as part of a group such as in “alkoxy”) means a straight or branched hydrocarbon chain containing the specified number of carbon atoms. For example, C₁₋₆alkyl means a straight or branched hydrocarbon chain containing at least 1 and at most 6 carbon atoms. Examples of alkyl include, but are not limited to: methyl (Me), ethyl (Et), n-propyl, isopropyl, n-butyl, s-butyl, isobutyl, t-butyl, n-hexyl and isohexyl. In a particular embodiment of the invention, the alkyl is C₁₋₃alkyl, i.e. methyl (Me), ethyl (Et), n-propyl, or isopropyl.

In a particular embodiment of the invention, “alkoxy” is C₁₋₃alkoxy, i.e. methoxy, ethoxy, n-propyloxy, or isopropyloxy.

The term ‘C₃₋₆cycloalkyl’, unless otherwise stated (e.g. by virtue of a different specified number of carbon atoms), means a closed 3 to 6 membered saturated carbocyclic ring, for example cyclopropyl, cyclobutyl, cyclopentyl, or cyclohexyl.

The term ‘halogen’ is used herein to mean, unless otherwise stated, a group which is fluorine, chlorine, bromine or iodine.

The term ‘aryl’ as used herein means a C₆₋₁₀ monocyclic or bicyclic hydrocarbon ring wherein at least one ring is aromatic. Examples of such groups include phenyl, naphthyl or tetrahydronaphthyl. In a particular embodiment, the aryl is phenyl.

It is to be understood that the present invention covers and discloses all possible combinations of particular, preferred, suitable, or other embodiments of groups or features (e.g. of A, R¹, R², R³, R⁴, R⁵, R⁶, R⁷, R⁸, R⁹, R¹⁰, X, n, m, p¹, p², p³ and/or p⁴), e.g. all possible combinations of embodiments of different groups or features, which embodiments are described herein.

In certain particular embodiments, A is aryl optionally substituted with 1 to 3 (e.g. 1 or 2) substituents, which may be the same or different, selected from the group consisting of halogen, C₁₋₆alkyl, —CF₃, C₁₋₄alkoxy (e.g. C₁₋₃alkoxy such as methoxy or isopropyloxy), C₁fluoroalkoxy (e.g. —OCF₃, —OCHF₂, or —OCH₂F), cyano, NR⁸R⁹, or pyridyl wherein the pyridyl is optionally substituted by methyl.

In certain particular embodiments, A is aryl optionally substituted by 1 to 3 (e.g. 1 or 2) substituents, which may be the same or different, and being halogen (e.g. fluorine, chlorine or bromine, in particular fluorine or chlorine), C₁₋₃alkyl (methyl, ethyl, n-propyl or isopropyl, in particular methyl or isopropyl, e.g. methyl), C₁₋₃alkoxy (e.g. methoxy or isopropyloxy), cyano, or NR⁸R⁹.

In certain more particular embodiments, A is phenyl optionally substituted by 1 to 3 (e.g. 1 or 2) substituents, which may be the same or different, and being halogen (e.g. fluorine, chlorine or bromine, in particular fluorine or chlorine), C₁₋₃alkyl (methyl, ethyl, n-propyl or isopropyl, in particular methyl or isopropyl, e.g. methyl), C₁₋₃alkoxy (e.g. methoxy or isopropyloxy), cyano, or NR⁸R⁹; or A is unsubstituted naphthyl (e.g. unsubstituted 1-naphthyl).

In certain still more particular embodiments, A is phenyl optionally substituted (e.g. substituted) by 1 to 3 (e.g. 1 or 2) substituents, which may be the same or different, and being halogen (e.g. fluorine, chlorine or bromine, in particular fluorine or chlorine), C₁₋₃alkyl (methyl, ethyl, n-propyl or isopropyl, in particular methyl or isopropyl, e.g. methyl), or NR⁸R⁹.

In certain particular embodiments, where A is optionally substituted phenyl, the phenyl is substituted by 1 to 3 (e.g. 1 or 2) substituents. Preferably, one of the phenyl substituent(s) is NR⁸R⁹. Preferably, the phenyl is substituted by one NR⁸R⁹ substituent and one fluorine, chlorine or methyl (e.g. chlorine or methyl) substituent.

Preferably, A is phenyl substituted by 1 to 3 (e.g. 1 or 2) substituents, which may be the same or different, and being halogen (e.g. fluorine, chlorine or bromine, in particular fluorine or chlorine), C₁₋₃alkyl (methyl, ethyl, n-propyl or isopropyl, in particular methyl or isopropyl, e.g. methyl), or NR⁸R⁹; provided that one of the phenyl substituent(s) is NR⁸R⁹. In particular, the phenyl can be substituted by one NR⁸R⁹ substituent and one fluorine, chlorine or methyl (e.g. chlorine or methyl) substituent.

In certain particular embodiments, R⁸ and R⁹ taken together are:

—(CH₂)₂—X—(CH₂)₂—, —(CH₂)ₚ¹—, —C(O)—(CH₂)ₚ²—, or —(CH₂)ₚ³—CH(R¹⁰)—(CH₂)ₚ⁴—.

In certain particular embodiments, X is O.

In certain particular embodiments, p¹ is 4 or 5.

In certain particular embodiments, p² is 3 or 4.

In certain particular embodiments, p⁴ is 2 or 3, and p³+p⁴ is 3 or 4.

In certain particular embodiments, R¹⁰ is OH or methoxy, more particularly OH.

Preferably, R⁸ and R⁹ taken together are —(CH₂)₂—O—(CH₂)₂—; —(CH₂)ₚ¹— wherein p¹ is 4 or 5; or —(CH₂)ₚ³—CH(R¹⁰)—(CH₂)ₚ⁴— wherein R¹⁰ is OH, p³ is 1 or 2, and p⁴ is 2 or 3, provided that p³+p⁴ is 3 or 4.

More preferably, R⁸ and R⁹ taken together are —(CH₂)₂—O—(CH₂)₂—.

In certain particular embodiments, A is aryl optionally substituted by 1 to 3 substituents, which may be the same or different, and being halogen (e.g. fluorine or chlorine) or C₁₋₆alkyl (e.g. methyl, ethyl or isopropyl, such as methyl).

In certain embodiments, A is unsubstituted naphthyl, e.g. unsubstituted 1-naphthyl.

In certain particular embodiments, A is phenyl optionally substituted by 1 to 3 substituents, which may be the same or different, and being fluorine, chlorine or methyl.

In certain embodiments, A is C₁₋₆alkyl (e.g. methyl, ethyl or isopropyl).

When A is phenyl optionally substituted by 1 to 3 (e.g. 1 or 2) substituents, which may be the same or different, and being halogen (e.g. fluorine, chlorine or bromine, in particular fluorine or chlorine), C₁₋₃alkyl (methyl, ethyl, n-propyl or isopropyl, in particular methyl or isopropyl, e.g. methyl), C₁₋₃alkoxy (e.g. methoxy or isopropyloxy), cyano, or NR⁸R⁹; then:

preferably A is substituted phenyl and has the following sub-formula (a):

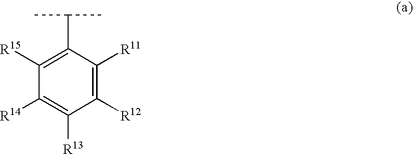

wherein:
R¹¹ is chlorine, C₁₋₃alkyl (e.g. methyl or isopropyl, in particular methyl), C₁₋₃alkoxy (e.g. methoxy or isopropyloxy), or cyano;
R¹² and R¹⁴ independently are hydrogen, halogen (e.g. fluorine, chlorine or bromine, in particular fluorine or chlorine), C₁₋₃alkyl (methyl, ethyl, n-propyl or isopropyl, in particular methyl or isopropyl, e.g. methyl), C₁₋₃alkoxy (e.g. methoxy or isopropyloxy), cyano, or NR⁸R⁹;
R¹³ is hydrogen or fluorine; and
R¹⁵ is hydrogen.

In certain particular embodiments, at least one of R¹² and R¹⁴ is other than hydrogen.

In these embodiments, preferably:

R¹¹ is chlorine, C₁₋₃alkyl (e.g. methyl or isopropyl), or cyano (in particular chlorine, methyl, or cyano);
R¹² and R¹⁴ independently are hydrogen, halogen (e.g. fluorine, chlorine or bromine, in particular fluorine or chlorine), or NR⁸R⁹; such that at least one of R¹² and R¹⁴ is other than hydrogen (preferably NR⁸R⁹);
R¹³ is hydrogen or fluorine (in particular hydrogen); and
R¹⁵ is hydrogen.

In these embodiments, preferably, one of R¹² and R¹⁴ is other than hydrogen (preferably NR⁸R⁹), and the other of R¹² and R¹⁴ is hydrogen.

In certain alternative particular embodiments,

R¹¹ is chlorine, C₁₋₃alkyl (e.g. methyl or isopropyl, in particular methyl), C₁₋₃alkoxy (e.g. methoxy or isopropyloxy), or cyano;
R¹³ is fluorine; and
R¹², R¹⁴ and R¹⁵ are hydrogen.

In these embodiments, preferably, R¹¹ is chlorine, methyl, or cyano.

In certain particular embodiments, R¹ is chlorine, fluorine or methyl, such as chlorine or fluorine.

Preferably, R¹ is chlorine.

In certain particular embodiments, R², R³ and R⁵ independently are hydrogen, fluorine, chlorine, —CF₃ or C₁₋₆alkyl (e.g. methyl).

In certain particular embodiments, R² is hydrogen, fluorine or chlorine (e.g. chlorine), —CF₃ or C₁₋₆alkyl (e.g. methyl).

In certain particular embodiments, R³ is hydrogen, fluorine or chlorine.

In the invention, R⁴ is hydrogen.

In certain particular embodiments, R⁵ is hydrogen, fluorine, chlorine, —CF₃, or C₁₋₆alkyl (e.g. methyl); in particular hydrogen, fluorine, chlorine, or methyl; more particularly hydrogen or chlorine. R⁵ can for example be hydrogen.

When R⁵ is fluorine, chlorine, —CF₃, cyano or C₁₋₆alkyl (i.e. when R⁵ is other than hydrogen), then preferably R² is hydrogen. For example, when R⁵ is fluorine, chlorine, or methyl, then preferably R² is hydrogen.

Preferably,

R² is hydrogen, chlorine, —CF₃ or methyl;
R³ is hydrogen, fluorine or chlorine; and
R⁵ is hydrogen, fluorine, chlorine, or methyl; such that at least one of R², R³ and R⁵ is other than hydrogen.

More preferably,

R¹ is chlorine;
R² is hydrogen, chlorine, —CF₃ or methyl;
R³ is hydrogen, fluorine or chlorine; and
R⁵ is hydrogen, fluorine, chlorine, or methyl;
such that at least one of R², R³ and R⁵ is other than hydrogen.

In one preferable embodiment of the invention, there is provided a compound of formula (I), or a pharmaceutically acceptable salt thereof, which is:
4-{[2-Chloro-3-(trifluoromethyl)phenyl]carbonyl}-1-(2-methylphenyl)-2-piperazinone (e.g. E1);
4-[(2,4-Dichlorophenyl)carbonyl]-1-(2-methylphenyl)-2-piperazinone (e.g. E2);
4-[(2-Chloro-4-fluorophenyl)carbonyl]-1-(2-methylphenyl)-2-piperazinone (e.g. E3);
4-[(2,3-Dichlorophenyl)carbonyl]-1-(2-methylphenyl)-2-piperazinone (e.g. E4);
4-[(2-Chloro-4-fluorophenyl)carbonyl]-1-(2-chlorophenyl)-2-piperazinone (e.g. E5);
1-(2-Dichlorophenyl)-4-[(2,3-dichlorophenyl)carbonyl]-2-piperazinone (e.g. E6);
1-(4-Chlorophenyl)-4-[(2,3-dichlorophenyl)carbonyl]-2-piperazinone (e.g. E7);
1-(2-Chlorophenyl)-4-[(2,4-dichlorophenyl)carbonyl]-2-piperazinone (e.g. E8);
1-(2-Chlorophenyl)-4-{[2-chloro-3-(trifluoromethyl)phenyl]carbonyl}-2-piperazinone (e.g. E9);
1-(2-Chloro-4-fluorophenyl)-4-{[2-chloro-3-(trifluoromethyl)phenyl]carbonyl}-2-piperazinone (e.g. E10);
1-(2-Chloro-4-fluorophenyl)-4-[(2,3-dichlorophenyl)carbonyl]-2-piperazinone (e.g. E11);
4-{[2-Chloro-3-(trifluoromethyl)phenyl]carbonyl}-1-(4-fluorophenyl)-2-piperazinone (e.g. E12);
4-[(2,3-Dichlorophenyl)carbonyl]-1-(4-fluorophenyl)-2-piperazinone (e.g. E13);
1-(3-Chlorophenyl)-4-[(2,3-dichlorophenyl)carbonyl]-2-piperazinone (e.g. E14);
1-[(2-Chloro-4-fluorophenyl)methyl]-4-[(2,3-dichlorophenyl)carbonyl]-2-piperazinone (e.g. E15);
4-[(2,3-Dichlorophenyl)carbonyl]-1-methyl-2-piperazinone (e.g. E16);
4-[(2,3-Dichlorophenyl)carbonyl]-1-ethyl-2-piperazinone (e.g. E1 7);
4-[(2,3-Dichlorophenyl)carbonyl]-1-(1-methylethyl)-2-piperazinone (e.g. E18);
4-[(2,3-Dichlorophenyl)carbonyl]-1-(2,4-difluorophenyl)-2-piperazinone (e.g. E19); or
4-[(2,4-dichlorophenyl)carbonyl]-1-(2,4-difluorophenyl)-2-piperazinone (e.g. E20).

In an alternative preferable embodiment of the invention, there is provided a compound of formula (I), or a pharmaceutically acceptable salt thereof, which is a compound of any one of Examples 21 to 87, or a pharmaceutically acceptable salt thereof.

Antagonists of P2X7 may be useful in preventing, treating, or ameliorating a variety of pain states (e.g. neuropathic pain, chronic inflammatory pain, and visceral pain), inflammation, or neurodegenerative diseases such as Alzheimer's disease. P2X7 antagonists may also constitute useful therapeutic agents in the management of rheumatoid arthritis or osteoarthritis.

Compounds or salts of the present invention which modulate P2X7 receptor function and are capable of antagonizing the effects of ATP at the P2X7 receptor (“P2X7 receptor antagonists”) may be competitive antagonists, inverse agonists, or negative allosteric modulators of P2X7 receptor function.

Certain compounds of formula (I) may in some circumstances form acid addition salts thereof. It will be appreciated that for use in medicine compounds of formula (I) may be used as salts, in which case the salts should be pharmaceutically acceptable. Pharmaceutically acceptable salts include those described by Berge, Bighley and Monkhouse, J. Pharm. Sci., 1977, 66, 1-19.

When a compound of formula (I) is basic, in one embodiment a pharmaceutically acceptable salt is prepared from a pharmaceutically acceptable acid such as an inorganic or organic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In a particular embodiment, the pharmaceutically acceptable acid is benzenesulfonic, camphorsulfonic, ethanesulfonic, hydrobromic, hydrochloric, methanesulfonic, nitric, phosphoric, sulfuric, or p-toluenesulfonic acid.

Examples of pharmaceutically acceptable salts include those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds of formula (I) or pharmaceutically acceptable salts thereof may be prepared in crystalline or non-crystalline form (e.g. in crystalline or amorphous solid form), and, in particular if crystalline, may optionally be solvated, e.g. as the hydrate. This invention includes within its scope solvates (e.g. hydrates) of compounds of formula (I) or pharmaceutically acceptable salts thereof, for example stoichiometric solvates (e.g. hydrates); as well as compounds or salts thereof containing variable amounts of solvent (e.g. water).

Certain compounds of formula (I) or salts thereof may be capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labelled compounds, which are identical to those recited in formula (I) or salts thereof, but for the fact that one or more atoms are replaced by an atom having an atomic mass or mass number different from the atomic mass or mass number most commonly found in nature. Examples of isotopes that can be incorporated into compounds or salts of the invention include isotopes of hydrogen, carbon, nitrogen, oxygen, phosphorous, fluorine, iodine, and chlorine, such as 3H, 11C, 14C, 18F, 123I and 125I.

Compounds of the present invention and pharmaceutically acceptable salts of said compounds that contain the aforementioned isotopes and/or other isotopes of other atoms are within the scope of the present invention. Isotopically-labelled compounds or salts of the present invention, for example those into which radioactive isotopes such as 3H, 14C are incorporated, are potentially useful in drug and/or substrate tissue distribution assays. Tritiated, i.e., 3H, and carbon-14, i.e., 14C, isotopes are for example optionally chosen for their (in some cases) ease of preparation and/or detectability. 11C and 8F isotopes are generally useful in PET (positron emission tomography), and 125I isotopes are generally useful in SPECT (single photon emission computerized tomography). PET and SPECT are generally useful in brain imaging. Further, substitution with heavier isotopes such as deuterium, i.e., 2H, can sometimes afford certain effects resulting from greater metabolic stability, for example increased in vivo half-life or reduced dosage requirements and, hence, may be chosen in some circumstances. Isotopically labelled compounds of formula (I) or salts thereof of this invention are in one embodiment and in some cases prepared by carrying out the procedures disclosed in the Schemes and/or in the Examples below, by substituting a readily available isotopically labelled reagent for a non-isotopically labelled reagent.

A further particular aspect of the invention provides a compound of formula (I) or a pharmaceutically acceptable salt thereof which is not a radioactive isotopically-labelled compound or salt. In a particular embodiment, the compound or salt is not an isotopically-labelled compound or salt.

### Preparation of Compounds

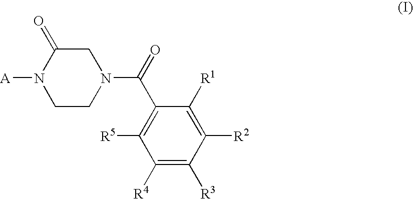

Compounds of formula (I), wherein the variables are as defined herein, and pharmaceutically acceptable salts thereof may be prepared by the methodology described hereinafter, constituting a further aspect of this invention.

According to a further aspect of the invention, a process for preparing a compound of formula (I) or a pharmaceutically acceptable salt thereof comprises step (a), (b), (c) or (d), as described below;

and optionally preparing a pharmaceutically acceptable salt of the compound.

(a) Preparation of a compound of formula (I) by coupling of a compound of general formula (4) with a carboxylic acid of general formula (5) (or an activated derivative thereof) (see Scheme 1, Scheme 3, Scheme 4 and/or Scheme 5) wherein A, R¹, R², R³, R⁴, and R⁵ are as defined herein. Compounds (4) and (5) are optionally protected.

(b) Preparation of a compound of formula (I) by reacting a compound of general formula (6) with a compound of general formula (7) (see Scheme 2) wherein A, R¹, R², R³, R⁴, and R⁵ are as defined herein. Compounds (6) and (7) are optionally protected.

(c) Deprotecting a compound of formula (I) which is protected. Examples of protecting groups and the means for their removal can be found in T. W. Greene and P. G. M. Wuts ‘Protective Groups in Organic Synthesis’ (Wiley-Interscience, 4^{th} ed., 2006).

(d) Interconversion of compounds of formula (I) to other compounds of formula (I). Examples of conventional interconversion procedures include epimerisation, oxidation, reduction, alkylation, aromatic substitution, nucleophilic substitution, amide coupling and ester hydrolysis.

Representative methods for the preparation of compounds of formula (I) are shown in Schemes 1 to 5 below:

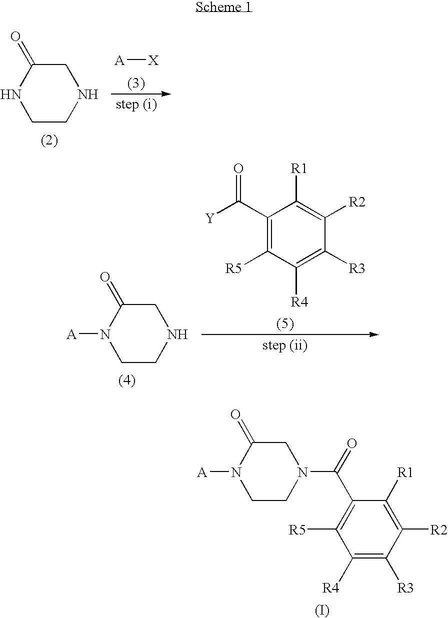

Step (i) typically comprises coupling of compound (2) with a compound of formula (3) wherein A represents a phenyl or a monocyclic heteroaryl group and X represents a suitable leaving group such as halogen (e.g. bromine or iodine) in the presence of a copper (I) salt, such as copper (I) iodide, in the presence of an amine ligand such as N,N′-dimethyl-1,2-cyclohexane diamine and a base such as potassium phosphate, in an appropriate solvent such as 1,4-dioxane, at an appropriate temperature such as reflux.

Step (ii) typically comprises coupling of a compound of formula (4) with a carboxylic acid of formula (5) (where Y═OH) in the presence of an activating agent, such as water soluble carbodiimide and a suitable base such as N,N-dimethylamino-4-pyridinamine, in a suitable solvent such as dichloromethane and at a suitable temperature e.g. between 0° C. and room temperature.

Alternatively, the compound of formula (5) may be employed as an activated derivative (e.g. acid chloride) and under such circumstances step (ii) typically comprises treatment of said activated derivative (5) (Y═Cl) with compound (4) in the presence of a suitable base such as N-ethyl-N-(1-methylethyl)-2-propanamine, in a suitable solvent such as dichloromethane and at a suitable temperature e.g. between 0° C. and room temperature.

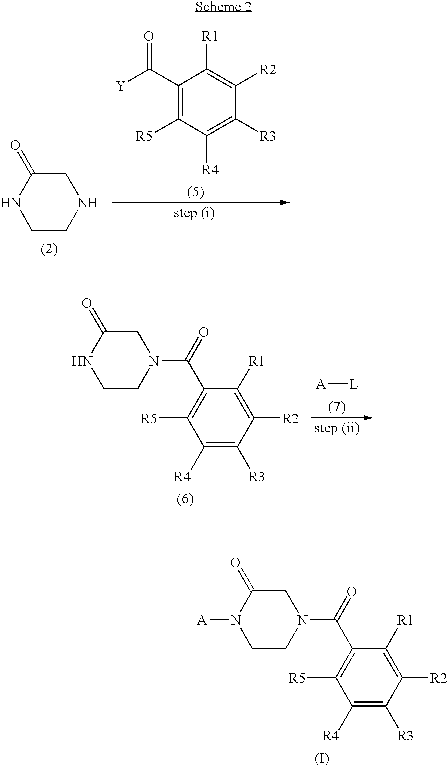

Step (i) typically comprises reactions analogous to those described in Scheme 1 step (ii).

Step (ii) typically comprises reacting a compound of formula (6) with a compound of formula (7), wherein A represents a C₁₋₆alkyl group or —CH₂—R⁶, and L represents a suitable leaving group such as a halogen atom (e.g. bromine or iodine), in the presence of a suitable base such as sodium hydride, in a suitable solvent such as N,N-dimethylformamide and at a suitable temperature e.g. between 0° C. and room temperature.

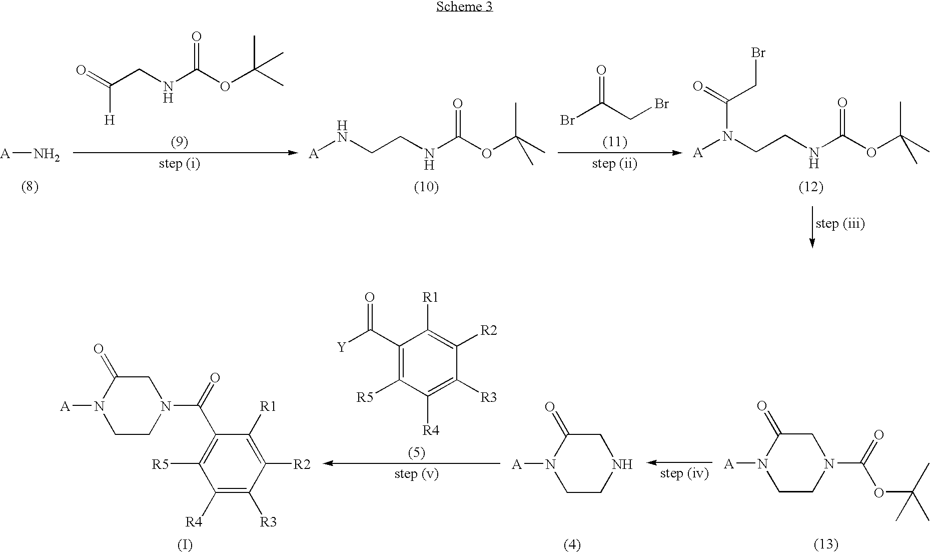

Step (i) typically comprises treatment of compound (8) with compound (9) with a suitable reducing agent such as sodium triacetoxyborohydride and a suitable dehydrating agent such as 4 Å molecular sieves in a suitable solvent such as dichloromethane and at a suitable temperature such as 0° C. or room temperature.

Step (ii) typically comprises treatment of compound (10) with a suitable reagent such as bromoacetyl bromide (11), with a suitable base such as sodium hydroxide, in a suitable solvent such as dichloromethane and at a suitable temperature such as 0° C. or room temperature.

Step (iii) typically comprises treatment of compound (12) with a suitable base such as potassium carbonate in a suitable solvent such as N,N-dimethylformamide and at a suitable temperature such as 50° C. or room temperature.

Step (iv) typically comprises treatment of compound (13) with a suitable acid such as hydrochloric acid, in a suitable solvent such as 1,4-dioxane and at a suitable temperature such as room temperature.

Step (v) typically comprises reactions analogous to those described in Scheme 1 step (ii).

Compounds of the general formulae (2), (3), (5), (7), (8), (9) and (11) are typically either available from commercial sources or can be prepared by a person skilled in the art using methods described in the chemical literature (or using analogous methods).

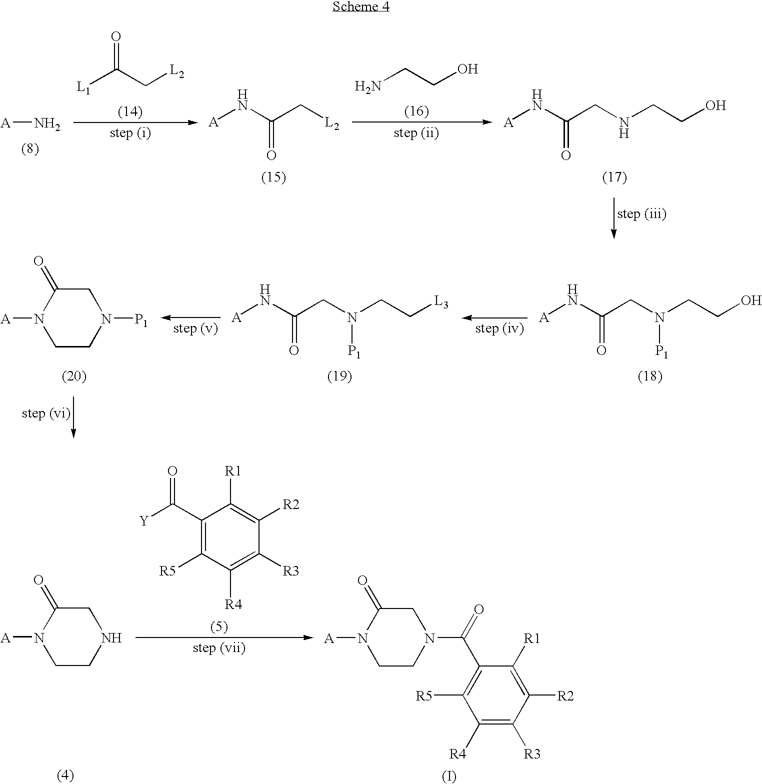

Step (i) typically comprises treatment of compound (8) with compound (14), in which L₁ and L₂ represent suitable leaving groups such as chlorine, in the presence of a suitable base such as potassium carbonate in a suitable solvent such as tetrahydrofuran and at a suitable temperature such as 5° C. or room temperature.

Step (ii) typically comprises treatment of compound (15) with compound (16), in a suitable solvent such as tetrahydrofuran and at a suitable temperature such as between 5° C. and 50° C.

Step (iii) typically comprises protecting compound (17) with a suitable amine protecting group (P₁), such as t-butoxy carbamoyl. Examples of protecting groups and the means for their removal can be found in T. W. Greene and P. G. M. Wuts ‘Protective Groups in Organic Synthesis’ (Wiley-Interscience, 4^{th} ed., 2006).

Step (iv) typically comprises conversion of the hydroxyl group of compound (18) into a suitable leaving group (L₃), such as mesylate, using a suitable reagent, such as methane sulfonyl chloride in the presence of a suitable base such as triethylamine, in a suitable solvent such as dichloromethane and at a suitable temperature such as room temperature.

Step (v) typically comprises treatment of compound (19) with a suitable base such as sodium hydride in a suitable solvent such as N,N-dimethylformamide at a suitable temperature such as room temperature.

Step (vi) typically comprises deprotection of compound (20) with a suitable reagent such as hydrochloric acid, in a suitable solvent such as 1,4-dioxane and at a suitable temperature such as room temperature.

Step (vii) typically comprises reactions analogous to those described in Scheme 1 step (ii).

Compounds of the general formulae (5), (8), (14), and (16) are typically either available from commercial sources or can be prepared by a person skilled in the art using methods described in the chemical literature (or using analogous methods).

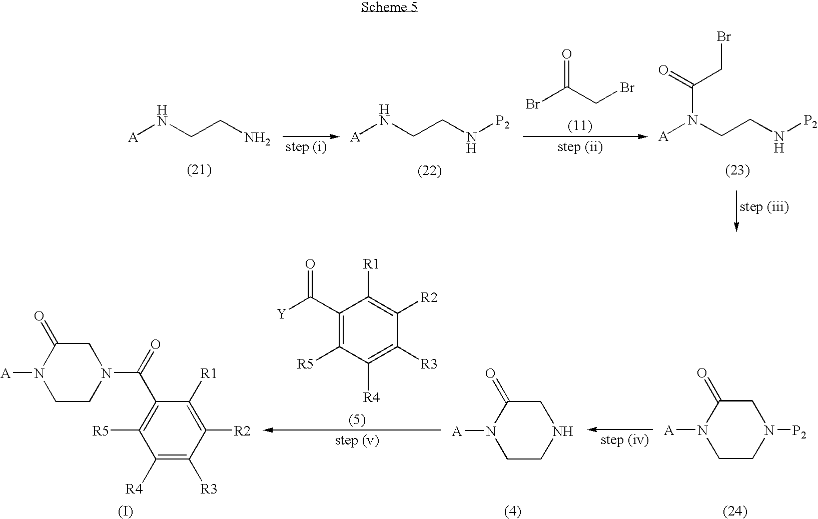

Step (i) typically comprises protecting compound (21) with a suitable amine protecting group (P₂), such as benzyloxy carbamoyl. Examples of protecting groups and the means for their removal can be found in T. W. Greene and P. G. M. Wuts ‘Protective Groups in Organic Synthesis’ (Wiley-Interscience, 4^{th} ed., 2006).

Step (ii) typically comprises treatment of compound (22) with a suitable reagent such as bromoacetyl bromide (11), with a suitable base such as sodium hydroxide, in a suitable solvent such as dichloromethane and at a suitable temperature such as 0° C. or room temperature.

Step (iii) typically comprises treatment of compound (23) with a suitable base such as potassium carbonate in a suitable solvent such as N,N-dimethylformamide and at a suitable temperature such as 50° C. or room temperature.

Step (iv) typically comprises deprotection of compound (24) with a reagents such as hydrogen over palladium on charcoal, in a suitable solvent such as a mixture of ethanol and acetic acid and at a suitable temperature such as room temperature.

Step (v) typically comprises reactions analogous to those described in Scheme 1 step (ii).

Compounds of the general formulae (5), (11), and (21) are typically either available from commercial sources or can be prepared by a person skilled in the art using methods described in the chemical literature (or using analogous methods).

Where relevant, pharmaceutically acceptable salts may be prepared conventionally by reaction with the appropriate acid or acid derivative.

### Clinical Indications

It is believed that, as the compounds or pharmaceutically acceptable salts of the present invention modulate P2X7 receptor function and are capable of antagonizing the effects of ATP at the P2X7 receptor (“P2X7 receptor antagonists”), they may be useful in the treatment of pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, cancer chemotherapy, headache, toothache and dysmenorrhea.

Chronic articular pain conditions include rheumatoid arthritis, osteoarthritis, rheumatoid spondylitis, gouty arthritis and juvenile arthritis.

In particular, the compounds or pharmaceutically acceptable salts of the present invention may be useful in the treatment or prevention of pain (e.g. inflammatory pain) in arthritis, such as pain (e.g. inflammatory pain) in rheumatoid arthritis or osteoarthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions which could potentially be treated by compounds or pharmaceutically acceptable salts of the present invention include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) or cyclooxygenase-2 (COX-2) inhibitors, cardiovascular diseases; neurodegenerative diseases and neurodegeneration, neurodegeneration following trauma, tinnitus, dependence on a dependence-inducing agent such as opioids (e.g. morphine), CNS (central nervous system) depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence. Depression and alcoholism could potentially also be treated by compounds or salts of the present invention.

Inflammation and the inflammatory conditions associated with said inflammation include skin conditions (e.g. sunburn, burns, eczema, dermatitis, allergic dermatitis, psoriasis), meningitis, ophthalmic diseases such as glaucoma, retinitis, retinopathies, uveitis and of acute injury to the eye tissue (e.g. conjunctivitis), inflammatory lung disorders (e.g. asthma, allergic rhinitis, respiratory distress syndrome, pigeon fancier's disease, farmer's lung, chronic obstructive pulmonary disease (COPD, which includes bronchitis and/or emphysema), or airways hyperresponsiveness); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, or gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

A bone disease characterised by abnormal bone metabolism or resorbtion may particular be rheumatoid arthritis or osteoarthritis, for potential treatment by compounds or pharmaceutically acceptable salts of the present invention.

Cardiovascular diseases include hypertension or myocardiac ischemia; atherosclerosis; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, dementia with Lewy bodies, Alzheimer's disease, Pick's disease, Huntingdon's chorea, Parkinson's disease and Creutzfeldt-Jakob disease, Amyotrophic Lateral Sclerosis (ALS) and motor neuron disease; in particular Alzheimer's disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment e.g. associated with ageing, particularly age associated memory impairment.

The neurodegenerative disease to be treated by the compound or salt can for example be degenerative dementia (in particular Alzheimer's disease), vascular dementia (in particular multi-infarct dementia), or mild cognitive impairment (MCI) e.g. MCI associated with ageing such as age associated memory impairment.

The compounds of formula (I) or pharmaceutically acceptable salts thereof may also be useful for neuroprotection and in the treatment of neurodegeneration following trauma such as stroke, cardiac arrest, pulmonary bypass, traumatic brain injury, spinal cord injury or the like.

The compounds or pharmaceutically acceptable salts of the present invention may also be useful in the treatment of malignant cell growth and/or metastasis, and myoblastic leukaemia.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, glomerulonephritis, particularly mesangial proliferative glomerulonephritis and nephritic syndrome.

It is to be understood that reference to treatment includes both treatment of established symptoms and prophylactic treatment, unless explicitly stated otherwise.

According to a further aspect of the invention, we therefore provide a compound of formula (I) or a pharmaceutically acceptable salt thereof for use in therapy and/or for use in human or veterinary medicine.

According to another aspect of the invention, we provide a compound of formula (I) or a pharmaceutically acceptable salt thereof for use in the treatment or prevention (e.g. treatment) of a condition which is mediated by P2X7 receptors, for example a condition or disease disclosed herein (in particular pain, inflammation, rheumatoid arthritis, osteoarthritis or a neurodegenerative disease, more particularly pain such as inflammatory pain, neuropathic pain or visceral pain), e.g. in a mammal such as a human or rodent e.g. human or rat e.g. human.

According to a further aspect of the invention, we provide a method of treating a human or animal (e.g. rodent e.g. rat) subject, for example a human subject, suffering from a condition which is mediated by P2X7 receptors, for example a condition or disease disclosed herein (in particular pain, inflammation, rheumatoid arthritis, osteoarthritis or a neurodegenerative disease, more particularly pain such as inflammatory pain, neuropathic pain or visceral pain), which comprises administering to said subject an effective amount of a compound of formula (I) or a pharmaceutically acceptable salt thereof.

According to a further aspect of the invention we provide a method of treating a human or animal (e.g. rodent e.g. rat) subject, for example a human subject, suffering from pain, inflammation, rheumatoid arthritis, osteoarthritis or a neurodegenerative disease (more particularly pain such as inflammatory pain, neuropathic pain or visceral pain), which method comprises administering to said subject an effective amount of a compound of formula (I) or a pharmaceutically acceptable salt thereof.

According to a yet further aspect of the invention we provide a method of treating a human or animal (e.g. rodent e.g. rat) subject, for example a human subject, suffering from inflammatory pain, neuropathic pain or visceral pain (e.g. pain, such as inflammatory pain, in arthritis (e.g. rheumatoid arthritis or osteoarthritis)) which method comprises administering to said subject an effective amount of a compound of formula (I) or a pharmaceutically acceptable salt thereof.

According to a further aspect of the invention we provide a method of treating a subject, for example a human subject, suffering from Alzheimer's disease which method comprises administering to said subject an effective amount of a compound of formula (I) or a pharmaceutically acceptable salt thereof.

According to another aspect of the invention, we provide the use of a compound of formula (I) or a pharmaceutically acceptable salt thereof for the manufacture of a medicament for the treatment or prevention (e.g. treatment) of a condition which is mediated by the action of P2X7 receptors, for example a condition or disease disclosed herein (in particular pain, inflammation, rheumatoid arthritis, osteoarthritis or a neurodegenerative disease; more particularly pain such as inflammatory pain, neuropathic pain or visceral pain; still more particularly pain, such as inflammatory pain, in arthritis (e.g. rheumatoid arthritis or osteoarthritis)), e.g. in a mammal such as a human or rodent e.g. human or rat e.g. human.

According to another aspect of the invention we provide the use of a compound of formula (I) or a pharmaceutically acceptable salt thereof for the manufacture of a medicament for the treatment or prevention (e.g. treatment) of pain, inflammation, rheumatoid arthritis, osteoarthritis or a neurodegenerative disease (in particular pain such as inflammatory pain, neuropathic pain or visceral pain; more particularly pain, such as inflammatory pain, in arthritis (e.g. rheumatoid arthritis or osteoarthritis)); e.g. in a mammal such as a human or rodent e.g. human or rat e.g. human.

According to another aspect of the invention we provide the use of a compound of formula (I) or a pharmaceutically acceptable salt thereof for the manufacture of a medicament for the treatment or prevention (e.g. treatment) of inflammatory pain, neuropathic pain or visceral pain (in particular inflammatory pain in arthritis such as rheumatoid arthritis or osteoarthritis), e.g. in a mammal such as a human or rodent e.g. human or rat e.g. human.

In one aspect of the invention we provide the use of a compound of formula (I) or a pharmaceutically acceptable salt thereof for the manufacture of a medicament for the treatment or prevention (e.g. treatment) of Alzheimer's disease, e.g. in a mammal such as a human or rodent e.g. human or rat e.g. human.

In order to use a compound of formula (I) or a pharmaceutically acceptable salt thereof for the treatment of humans and/or other mammals it is normally formulated in accordance with standard pharmaceutical practice as a pharmaceutical composition. Therefore in another aspect of the invention there is provided a pharmaceutical composition comprising a compound of formula (I), or a pharmaceutically acceptable salt thereof, adapted for use in human or veterinary medicine.

In order to use a compound of formula (I) or a pharmaceutically acceptable salt thereof in therapy, it will normally be formulated into a pharmaceutical composition in accordance with standard pharmaceutical practice. The present invention also provides a pharmaceutical composition, which comprises a compound of formula (I), or a pharmaceutically acceptable salt thereof, and a pharmaceutically acceptable carrier or excipient.

The pharmaceutical composition may be for use in a method of treatment or in a use or in a treatment or prevention, as described herein.

A pharmaceutical composition of the invention, which may be prepared by admixture, for example at ambient temperature and atmospheric pressure, is usually adapted for oral, parenteral or rectal administration. As such, the pharmaceutical composition may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain excipient(s), such as a binding agent, a filler, a tabletting lubricant, a disintegrant (e.g. tablet disintegrant) and/or an acceptable wetting agent. The tablets may be coated according to methods known in pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain additives such as suspending agents, emulsifying agents, non-aqueous vehicles (which may include edible oils), and/or preservatives, and/or, if desired, flavourings or colourants.

For parenteral administration, fluid unit dosage forms are for example prepared utilising a compound of the invention or pharmaceutically acceptable salt thereof and a sterile vehicle. In one embodiment, the compound or salt, depending on the vehicle and concentration used, is either suspended or dissolved in the vehicle. In preparing solutions, the compound or salt can be dissolved for injection and filter sterilised before filling into a suitable vial or ampoule and sealing. In one embodiment, an adjuvant(s) such as a local anaesthetic, a preservative and/or a buffering agent is or are dissolved in the vehicle. To enhance the stability, the composition can for example be frozen after filling into the vial and the water removed under vacuum. Parenteral suspensions are typically prepared in substantially the same manner, except that the compound or salt is typically suspended in the vehicle instead of being dissolved, and sterilization is not usually readily accomplished by filtration. The compound or salt can be sterilised e.g. by exposure to ethylene oxide before suspension in a sterile vehicle. In a particular embodiment, a surfactant or wetting agent is included in the composition to facilitate uniform distribution of the compound or salt of the invention.

In one embodiment, the composition contains from 0.1% to 99% (by weight of the composition), in particular from 0.1 to 60% or 1 to 60% or 10 to 60% by weight, of the active material (the compound or pharmaceutically acceptable salt of the invention), e.g. depending on the method of administration. The carrier(s) and/or excipient(s) contained in the composition can for example be present in from 1% to 99.9%, e.g. from 10% to 99%, by weight of the composition.

The dose of the compound or pharmaceutically acceptable salt thereof used in the treatment or prevention (e.g. treatment) of the aforementioned disorders/diseases/conditions may vary in the usual way with the seriousness of the disorders, the weight of the sufferer, and/or other similar factors. However, as a general guide, in one embodiment a suitable unit dose of 0.05 to 1000 mg, for example 0.05 to 200 mg, such as 20 to 40 mg, of the compound or pharmaceutically acceptable salt of the invention (measured as the compound), may be used. In one embodiment, such a unit dose is for administration once a day e.g. to a mammal such as a human; alternatively such a unit dose may be for administration more than once (e.g. twice) a day e.g. to a mammal such as a human. Such therapy may extend for a number of weeks or months.

### Combinations

Compounds of formula (I) or pharmaceutically acceptable salts thereof may be used in combination with other therapeutic agents, for example medicaments claimed to be useful in the treatment or prevention (e.g. treatment) of the above mentioned disorders.

Suitable examples of other such therapeutic agents may include a β2-agonist (also known as β2 adrenoceptor agonists; e.g. formoterol) and/or a corticosteroid (e.g. budesonide, fluticasone (e.g. as propionate or furoate esters), mometasone (e.g. as furoate), beclomethasone (e.g. as 17-propionate or 17,21-dipropionate esters), ciclesonide, triamcinolone (e.g. as acetonide), flunisolide, rofleponide and butixocort (e.g. as propionate ester), for the treatment of respiratory disorders (such as asthma and chronic obstructive pulmonary disease (COPD)), e.g. as described in WO 2007/008155 and/or WO 2007/008157.

A further therapeutic agent may include a 3-hydroxy-3-methylglutaryl coenzyme A (HMG CoA) reductase inhibitor (e.g. atorvastatin, fluvastatin, lovastatin, pravastatin, rosuvastatin, and simvastatin) for the treatment of cardiovascular disorders (such as atherosclerosis), e.g. as described in WO 2006/083214.

A further therapeutic agent may include a non-steroid anti-inflammatory drug (NSAID; e.g. ibuprofen, naproxen, aspirin, celecoxib, diclofenac, etodolac, fenoprofen, indomethacin, ketoprofen, ketoralac, oxaprozin, nabumetone, sulindac, tolmetin, rofecoxib, valdecoxib, lumaricoxib, meloxicam, etoricoxiband and parecoxib) for the treatment of an inflammatory disease or disorder (such as rheumatoid arthritis or osteoarthritis), e.g. as described in WO 2005/025571.

A further therapeutic agent may in particular include a tumour necrosis factor α (TNFα) inhibitor (e.g. Etanercept or an anti-TNFα antibody such as infliximab and adalimumab) (e.g. for parenteral administration such as subcutaneous or intravenous administration) for the treatment of an inflammatory disease or disorder (such as rheumatoid arthritis or osteoarthritis), e.g. as described in WO 2004/105798.

A further therapeutic agent may in particular include an anti-CD20 monoclonal antibody (e.g. for parenteral such as intravenous administration), such as ofatumumab (HuMax-CD20™, developed in part by Genmab AS) (e.g. ofatumumab for intravenous administration), rituximab, PRO70769, AME-133 (Applied Molecular Evolution), or hA20 (Immunomedics, Inc.); in particular ofatumumab or rituximab.

A further therapeutic agent may in particular include 2-hydroxy-5-[[4-[(2-pyridinylamino) sulfonyl]phenyl]azo]benzoic acid (sulfasalazine) for the treatment of an inflammatory disease or disorder (such as rheumatoid arthritis), e.g. as described in WO 2004/105797.

A further therapeutic agent may in particular include N-[4-[[(2,4-diamino-6-pteridinyl)methyl]methylamino]benzoyl]-L-glutamic acid (methotrexate) for the treatment of an inflammatory disease or disorder (such as rheumatoid arthritis), e.g. as described in WO 2004/105796.

A further therapeutic agent may include an inhibitor of pro TNFα convertase enzyme (TACE) for the treatment of an inflammatory disease or disorder (such as rheumatoid arthritis), e.g. as described in WO 2004/073704.

A further therapeutic agent may include:

a) sulfasalazine;
b) a statin (e.g. for oral administration), such as atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin or itavastatin;
c) a glucocorticoid agent (e.g. for oral or skin-topical administration), such as dexamethasone, methylprednisolone, prednisolone, prednisone and hydrocortisone;
d) an inhibitor of p38 kinase (e.g. for oral administration);
e) an anti-IL-6-receptor antibody e.g. an anti-IL-6-receptor monoclonal antibody (e.g. for parenteral such as intravenous administration);
f) anakinra;
g) an anti-IL-1 monoclonal antibody (e.g. for parenteral such as intravenous administration);
h) an inhibitor of JAK3 protein tyrosine kinase;
i) an anti-macrophage colony stimulation factor (M-CSF) monoclonal antibody; or
j) an anti-CD20 monoclonal antibody (e.g. for parenteral such as intravenous administration), such as ofatumumab (HuMax-CD20™, developed in part by Genmab AS) (e.g. ofatumumab for intravenous administration), rituximab, PRO70769, AME-133 (Applied Molecular Evolution), or hA20 (Immunomedics, Inc.), in particular ofatumumab or rituximab;
for the treatment of an IL-1 mediated disease (such as rheumatoid arthritis), e.g. as described in WO 2006/003517.

When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable salt thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable salt thereof is used in combination with a second therapeutic agent active against the same disease state the dose of each compound may differ from that when the compound is used alone.

The following Examples illustrate the preparation of compounds or salts of the invention but are not intended to be limiting.

### EXAMPLES

The general methods (a)-(d), along with the synthetic methods outlined in Schemes 1 to 5 above, for the preparation of compounds or salts of the present invention, are further illustrated by the following non-limiting examples.

Abbreviations, some of which may be used herein, include the following:
BINAP 2,2′-bis(diphenylphosphino)-1,1′-binaphthalene
BOC tert-butyl oxy carbonyl
BOC₂O di tert-butyl carbonate
DMSO dimethyl sulfoxide
DCM dichloromethane
DMAP 4-dimethylaminopyridine
DMF N,N-dimethylformamide
DIPEA N,N-diisopropylethyl amine (ⁱPr₂NEt)
EDC 1-(3-Dimethylaminopropyl)-3-ethylcarbodiimide hydrochloride
EtOAc ethyl acetate
Et₂O diethyl ether
EtOH ethanol
HEPES 4-(2-hydroxyethyl)-1-piperazine-1-ethanesulfonic acid

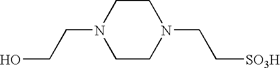

IPA isopropanol (isopropyl alcohol)
MeCN acetonitrile
MeOH methanol
MDAP mass directed autoprep HPLC
THF Tetrahydrofuran
TFA Trifluoroacetic acid
eq equivalents
HPLC high performance liquid chromatography
h hours
min minutes
LCMS or LC/MS liquid chromatography/mass spectroscopy
NMR nuclear magnetic resonance
SCX solid phase extraction (SPE) column with benzene sulfonic acid residues immobilised on the solid phase (eg. IST Isolute™ columns). When eluting with ammonia/methanol, it is thought that compounds isolated by SCX are usually in the free base form.
TLC thin layer chromatography
RT room temperature (ambient temperature); this is usually in the range of about 18 to about 25° C., or a sub-range within this range, except as disclosed herein.

### Example 1

### 4-{[2-Chloro-3-(trifluoromethyl)phenyl]carbonyl}-1-(2-methyl phenyl)-2-piperazinone (E1)

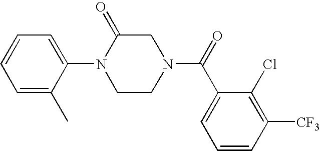

A solution of 1-(2-methylphenyl)-2-piperazinone (150 mg, 0.79 mmol, prepared as described below), 1-(3-dimethylaminopropyl)-3-ethylcarbodiimide hydrochloride (242 mg, 1.26 mmol), and N,N-dimethyl-4-pyridinamine (385 mg, 3.15 mmol) in dichloromethane (4 ml) was stirred at room temperature under argon. 2-Chloro-3-(trifluoromethyl)benzoic acid (177 mg, 0.79 mmol) was added portionwise, and the reaction mixture was left to stir at room temperature under argon overnight. Dichloromethane and aqueous 3N citric acid were added and the mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product was purified by flash-silica gel chromatography, eluting with 30-100% ethyl acetate in isohexane, to give the product 4-{[2-chloro-3-(trifluoromethyl)phenyl]carbonyl}-1-(2-methylphenyl)-2-piperazinone (138 mg) as a white solid. LC/MS [M+H]⁺=397, retention time=2.77 minutes.

The 1-(2-methylphenyl)-2-piperazinone used in the above procedure was prepared as follows:

A suspension of 2-piperazinone (1.65 g, 16.5 mmol), 1-iodo-2-methylbenzene (2.09 ml, 16.5 mmol), copper(I) iodide (0.628 g, 3.3 mmol), N,N′-dimethyl-1,2-cyclohexanediamine (1.04 ml, 6.6 mmol) and potassium phosphate (10.49 g, 49.4 mmol) in 1,4-dioxane (20 ml) was heated at reflux (100° C.) under argon for 20 hours. The mixture was allowed to cool to room temperature and then diluted with dichloromethane and water. The mixture was extracted into dichloromethane (×3), and then the combined organic extracts were washed with water (×4) and dried over magnesium sulphate. The solvent was evaporated in vacuo and then the crude product was purified further by column chromatography on flash-silica gel, eluting with 0-10% methanol in dichloromethane. The relevant fractions were combined and the solvent was evaporated in vacuo to give a yellow oil which was purified again by column chromatography on flash-silica gel, eluting with 5% methanol in dichloromethane to give 1-(2-methylphenyl)-2-piperazinone (1.33 g) as a dark yellow oil, which was used without further purification. LC/MS [M+H]⁺=191, retention time=0.85 minutes.

### Example 2

### 4-[(2,4-Dichlorophenyl)carbonyl]-1-(2-methylphenyl)-2-piperazinone (E2)

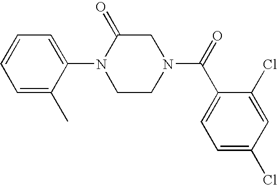

4-[(2,4-Dichlorophenyl)carbonyl]-1-(2-methylphenyl)-2-piperazinone was prepared in a manner analogous to that described above for the synthesis of 4-{[2-chloro-3-(trifluoromethyl)phenyl]carbonyl}-1-(2-methylphenyl)-2-piperazinone in Example 1 but 2,4-dichlorobenzoic acid was used in place of the 2-chloro-3-(trifluoromethyl)benzoic acid.

LC/MS [M+H]⁺=363, retention time=2.71 minutes.

### Example 3

### 4-[(2-Chloro-4-fluorophenyl)carbonyl]-1-(2-methylphenyl)-2-piperazinone (E3)

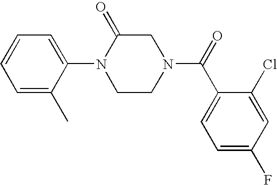

A solution of 1-(2-methylphenyl)-2-piperazinone (146 mg, 0.77 mmol, prepared as described above for Example 1) and N-ethyl-N-(1-methylethyl)-2-propanamine (0.20 ml, 1.15 mmol) in dichloromethane (4 ml) was stirred at 0° C. under argon. 2-Chloro-4-fluorobenzoyl chloride (148 mg, 0.77 mmol) was added portionwise. The mixture was allowed to warm to room temperature and was stirred over the weekend. Dichloromethane and aqueous 3N citric acid were added and the resulting mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product purified by flash-silica gel chromatography, eluting with 0-60% ethyl acetate in isohexane, to give 4-[(2-chloro-4-fluorophenyl)carbonyl]-1-(2-methylphenyl)-2-piperazinone (54 mg) as a white solid. LC/MS [M+H]⁺=347, retention time=2.51 minutes.

### Example 4

### 4-[(2,3-Dichlorophenyl)carbonyl]-1-(2-methylphenyl)-2-piperazinone (E4)

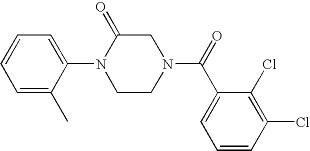

1-(2-Methylphenyl)-2-piperazinone (72 mg, 0.38 mmol, prepared as described below) was suspended in dichloromethane (4 ml). Triethylamine (0.06 ml, 0.45 mmol) and 2,3-dichlorobenzoyl chloride (95 mg, 0.45 mmol) were added and the mixture was stirred at room temperature for 16 hours. The reaction mixture was concentrated under vacuum and purified by mass-directed automated HPLC. Product-containing fractions were concentrated under vacuum to give 4-[(2,3-dichlorophenyl)carbonyl]-1-(2-methylphenyl)-2-piperazinone (79 mg) as an off-white solid. LC/MS [M+H]⁺=363, retention time=2.63 minutes.

The 1-(2-methylphenyl)-2-piperazinone used in the above procedure was prepared as follows:

(i) N-Boc-2-aminoacetaldehyde (2 g, 12.6 mmol) was dissolved in dichloromethane (50 ml), and 4 Angstrom molecular sieves (0.3 g) were added. The mixture was cooled to 0° C. and acetic acid (2.16 ml, 37.7 mmol), sodium triacetoxyborohydride (3.99 g, 18.9 mmol) and o-toluidine (1.43 ml, 13.2 mmol) were added. The dark brown mixture was warmed to room temperature and stirred for 15 hours. The reaction was quenched with saturated aqueous sodium bicarbonate (30 ml) and stirred for 10 minutes. The organic layer was separated using a hydrophobic frit and concentrated under vacuum. The crude material was purified by automated flash-silica gel column chromatography (Biotage SP4), eluting with a 0-70% gradient of ethyl acetate in hexane, to give 1,1-dimethylethyl {2-[(2-methylphenyl)amino]ethyl}carbamate (2.31 g) as an orange oil.
(ii) 1,1-Dimethylethyl {2-[(2-methylphenyl)amino]ethyl}carbamate (2.31 g, 9.2 mmol) was dissolved in dichloromethane (30 ml) and cooled to 0° C. 2N Sodium hydroxide (5.8 ml, 11.6 mmol) was added and the biphasic mixture stirred vigorously. Bromoacetyl bromide (0.88 ml, 10.2 mmol) was added and the mixture was stirred at 0° C. for 30 minutes. After this time, the mixture was warmed to room temperature and stirred for a further 90 minutes. Water (20 ml) was added and the mixture stirred. The organic layer was separated using a hydrophobic frit and concentrated under vacuum to give 1,1-dimethylethyl {2-[(bromoacetyl)(2-methylphenyl)amino]ethyl}carbamate (3.33 g) as an orange oil, which was used without further purification. LC/MS [M-BOC+H]⁺=271, 273.
(iii) 1,1-Dimethylethyl {2-[(bromoacetyl)(2-methylphenyl)amino]ethyl}carbamate (3.33 g, 9.0 mmol) was dissolved in N,N-dimethylformamide (50 ml) and potassium carbonate (3.71 g, 26.9 mmol) was added. The orange suspension was stirred at room temperature for 24 hours, and was then warmed to 50° C. and stirred for a further 24 hours. The reaction mixture was concentrated under vacuum. The residue was partitioned between dichloromethane (80 ml) and water (80 ml), and the organic layer was separated using a hydrophobic frit and concentrated under vacuum. The residue was purified by automated flash-silica gel column chromatography (Biotage SP4), eluting with a gradient of 0-50% ethyl acetate in hexane to give 1,1-dimethylethyl 4-(2-methylphenyl)-3-oxo-1-piperazinecarboxylate (1.08 g) as an orange oil. LC/MS [M+H]⁺=291.
(iv) 1,1-Dimethylethyl 4-(2-methylphenyl)-3-oxo-1-piperazinecarboxylate (1.08 g, 3.7 mmol) was dissolved in hydrochloric acid in dioxane (4N) (9 ml, 36.0 mmol) and stirred at room temperature for 1 hour. The reaction was concentrated under vacuum and the residue was dissolved in methanol (˜10 ml) and loaded onto a pre-conditioned SCX cartridge and washed with methanol (80 ml), followed by 2M ammonia in methanol (80 ml). The ammonia fractions were concentrated under vacuum to give 1-(2-methylphenyl)-2-piperazinone (0.604 g) as an orange oil which was used without further purification.

### Example 5

### 4-[(2-Chloro-4-fluorophenyl)carbonyl]-1-(2-chlorophenyl)-2-piperazinone (E5)

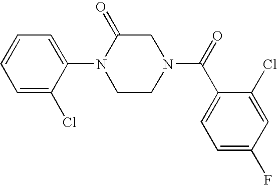

1-(2-Chlorophenyl)-2-piperazinone hydrochloride (100 mg, 0.41 mmol) and N-ethyl-N-(1-methylethyl)-2-propanamine (0.11 ml, 0.61 mmol) were added together in dichloromethane (4 ml) at 0° C. Subsequently 2-chloro-4-fluorobenzoyl chloride (86 mg, 0.45 mmol) was added portionwise. The reactants were left under argon and in an icebath and allowed to return to room temperature whilst being stirred constantly overnight. Dichloromethane and aqueous 3N citric acid were added and the mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product was purified by flash-silica gel chromatography, eluting with 30-100% ethyl acetate in isohexane, to give 4-[(2-chloro-4-fluorophenyl)carbonyl]-1-(2-chlorophenyl)-2-piperazinone (79 mg) as a white solid. LC/MS [M+H]⁺=367, retention time=2.54 minutes.

### Example 6

### 1-(2-Dichlorophenyl)-4-[(2,3-dichlorophenyl)carbonyl]-2-piperazinone (E6)

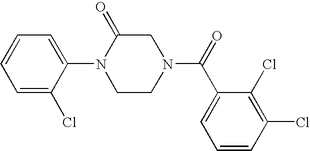

1-(2-Dichlorophenyl)-4-[(2,3-dichlorophenyl)carbonyl]-2-piperazinone was prepared in a manner analogous to that described above for the synthesis of 4-[(2-chloro-4-fluorophenyl)carbonyl]-1-(2-chlorophenyl)-2-piperazinone in Example 5 but 2,3-dichlorobenzoyl chloride was used in place of the 2-chloro-4-fluorobenzoyl chloride.

LC/MS [M+H]⁺=385, retention time=2.70 minutes.

### Example 7

### 1-(4-Chlorophenyl)-4-[(2,3-dichlorophenyl)carbonyl]-2-piperazinone (E7)

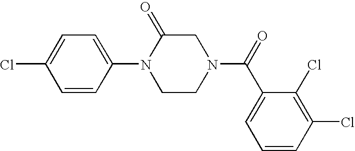

1-(4-Chlorophenyl)-4-[(2,3-dichlorophenyl)carbonyl]-2-piperazinone was prepared in a manner analogous to that described above for the synthesis of 4-[(2-chloro-4-fluorophenyl)carbonyl]-1-(2-chlorophenyl)-2-piperazinone in Example 6 but 1-(4-chlorophenyl)-2-piperazinone hydrochloride was used in place of the 1-(2-chlorophenyl)-2-piperazinone hydrochloride. LC/MS [M+H]⁺=385, retention time=2.81 minutes.

### Example 8

### 1-(2-Chlorophenyl)-4-[(2,4-dichlorophenyl)carbonyl]-2-piperazinone (E8)

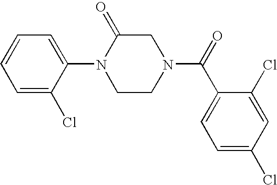

A solution of 1-(2-chlorophenyl)-2-piperazinone hydrochloride (100 mg, 0.41 mmol), N-(3-dimethylaminopropyl)-N′-ethyl-carbodiimide (0.115 ml, 0.647 mmol), and N,N-dimethyl-4-pyridinamine (198 mg, 1.62 mmol) in dichloromethane (4 ml) was stirred at room temperature under argon. 2,4-Dichlorobenzoic acid (77 mg, 0.41 mmol) was added portionwise and the reaction mixture was left to stir at room temperature under argon overnight. Dichloromethane and aqueous 3N citric acid were added and the mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product purified by flash-silica gel chromatography, eluting with 30-100% ethyl acetate in isohexane, to give 1-(2-chlorophenyl)-4-[(2,4-dichlorophenyl)carbonyl]-2-piperazinone (75 mg) as a white solid. LC/MS [M+H]⁺=385, retention time=2.74 minutes.

### Example 9

### 1-(2-Chlorophenyl)-4-{[2-chloro-3-(trifluoromethyl)phenyl]carbonyl}-2-piperazinone (E9)

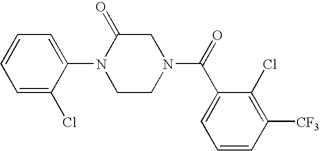

1-(2-Chlorophenyl)-4-{[2-chloro-3-(trifluoromethyl)phenyl]carbonyl}-2-piperazinone was prepared in a manner analogous to that described above for the synthesis of 1-(2-chlorophenyl)-4-[(2,4-dichlorophenyl)carbonyl]-2-piperazinone in Example 8 but 2-chloro-3-(trifluoromethyl)benzoic acid was used in place of the 2,4-dichlorobenzoic acid. LC/MS [M+H]⁺=417, retention time=2.79 minutes.

### Example 10

### 1-(2-Chloro-4-fluorophenyl)-4-{[2-chloro-3-(trifluoromethyl)phenyl]carbonyl}-2-piperazinone (E10)

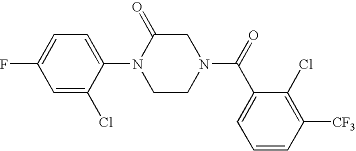

A solution of 1-(2-chloro-4-fluorophenyl)-2-piperazinone (75 mg, 0.33 mmol, prepared as described below), 1-(3-dimethylaminopropyl)-3-ethylcarbodiimide hydrochloride (101 mg, 0.525 mmol), and N,N-dimethyl-4-pyridinamine (160 mg, 1.31 mmol) in dichloromethane (4 ml) was stirred at room temperature under argon. 2-chloro-3-(trifluoromethyl)benzoic acid (74 mg, 0.33 mmol) was added portionwise and the reaction mixture was left to stir at room temperature under argon overnight. Dichloromethane and aqueous 3N citric acid were added and the product was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product was purified by flash-silica gel chromatography eluting with 30-100% ethyl acetate in isohexane. The resulting product was repurified by flash-silica gel chromatography, eluting with 30-70% ethyl acetate in isohexane, to give the product 1-(2-chloro-4-fluorophenyl)-4-{[2-chloro-3-(trifluoromethyl)phenyl]carbonyl}-2-piperazinone (18 mg), as a white solid.

LC/MS [M+H]⁺=435, retention time=2.84 minutes.

The 1-(2-chloro-4-fluorophenyl)-2-piperazinone used in the above procedure was prepared as follows: A suspension of 2-piperazinone (1.5 g, 15.0 mmol), 2-chloro-4-fluoro-1-iodobenzene (3.8 g, 15.0 mmol), copper(I) iodide (0.57 g, 3.0 mmol), N,N′-dimethyl-1,2-cyclohexanediamine (1.3 g, 6.0 mmol), and potassium phosphate (6.4 g, 44.9 mmol) in 1,4-dioxane (20 ml) was heated at reflux (100° C.) under argon for 20 hours. The mixture was allowed to cool to room temperature and then diluted with dichloromethane and 0.88M ammonia diluted in water (1:5). The mixture was extracted into dichloromethane (×3), and then the combined organic extracts were washed with water (×2) and dried over magnesium sulphate. The solvent was evaporated in vacuo and the crude product was purified by flash-silica gel chromatography, eluting with 5% 2M ammonia in methanol in dichloromethane, to give the product 1-(2-chloro-4-fluorophenyl)-2-piperazinone (250 mg) as a brown oil which was used without further purification. LC/MS [M+H]⁺=229.

### Example 11

### 1-(2-Chloro-4-fluorophenyl)-4-[(2,3-dichlorophenyl)carbonyl]-2-piperazinone (E11)

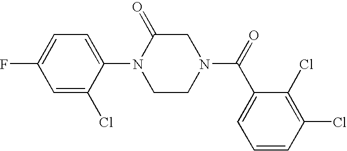

1-(2-Chloro-4-fluorophenyl)-2-piperazinone (100 mg, 0.44 mmol, prepared as described above for Example 10) and N-ethyl-N-(1-methylethyl)-2-propanamine (0.12 ml, 0.70 mmol) were added together in dichloromethane (4 ml) at 0° C. Subsequently 2,3-dichlorobenzoyl chloride (101 mg, 0.48 mmol) was added portionwise. The mixture was left under argon and in an icebath and allowed to return to room temperature whilst being stirred constantly overnight. Dichloromethane and aqueous 3N citric acid were added and the mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product was purified by flash-silica gel chromatography, eluting with 30-100% ethyl acetate in isohexane, to give 1-(2-chloro-4-fluorophenyl)-4-[(2,3-dichlorophenyl)carbonyl]-2-piperazinone (47 mg) as a white solid. LC/MS [M+H]⁺=403, retention time=2.76 minutes.

### Example 12

4-{[2-Chloro-3-(trifluoromethyl)phenyl]carbonyl}-1-(4-fluorophenyl)-2-piperazinone (E12)

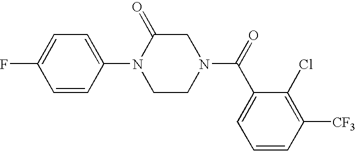

A solution of 1-(4-fluorophenyl)-2-piperazinone (100 mg, 0.52 mmol, prepared as described below), 1-(3-dimethylaminopropyl)-3-ethylcarbodiimide hydrochloride (158 mg, 0.82 mmol), and N,N-dimethyl-4-pyridinamine (252 mg, 2.06 mmol) in dichloromethane (4 ml) was stirred at room temperature under argon. 2-Chloro-3-(trifluoromethyl)benzoic acid (116 mg, 0.52 mmol) was added portionwise and the mixture was left overnight. Dichloromethane and aqueous 3N citric acid were then added and the mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo and the crude product was purified by flash-silica gel chromatography, eluting with 30-70% ethyl acetate in isohexane, to give 4-{[2-chloro-3-(trifluoromethyl)phenyl]carbonyl}-1-(4-fluorophenyl)-2-piperazinone (101 mg) as a white solid. LC/MS [M+H]⁺=401, retention time=2.70 minutes.

The 1-(4-fluorophenyl)-2-piperazinone used in the above procedure was prepared as follows:

A mixture of 2-piperazinone (1.5 g, 15.0 mmol), 1-fluoro-4-iodobenzene (3.5 ml, 30.0 mmol), copper(I) iodide (0.57 g, 3.0 mmol), N,N′-dimethyl-1,2-cyclohexanediamine (0.95 ml, 6.0 mmol) and potassium phosphate (9.5 g, 44.9 mmol) in 1,4-dioxane (20 ml) was heated at reflux (110° C.) under argon for 24 hours. The mixture was allowed to cool to room temperature and then diluted with methanol, and filtered through a pad of celite, washing with methanol. The filtrate was evaporated in vacuo and the resulting residue was dissolved in dichloromethane and 0.88 aqueous ammonia solution (˜5 ml) in water (˜30 ml). The mixture was then extracted into dichloromethane (×3), and the combined organic extracts were washed with water (×1) and dried over magnesium sulphate. The solvent was evaporated in vacuo and the crude product was purified further by column chromatography on flash-silica gel, eluting with 0-10% methanol in dichloromethane. The relevant fractions were combined and the solvent was evaporated in vacuo. The residue was purified by SCX, eluting first with methanol and then with 2M ammonia in methanol. The basic fractions were combined and the solvent was evaporated in vacuo to give crude product which was purified again by flash-silica gel column chromatography, eluting with 20% 2M ammonia in methanol in dichloromethane, to give 1-(4-fluorophenyl)-2-piperazinone (560 mg) as a colourless solid. LC/MS [M+H]⁺=195.

### Example 13

### 4-[(2,3-Dichlorophenyl)carbonyl]-1-(4-fluorophenyl)-2-piperazinone (E13)

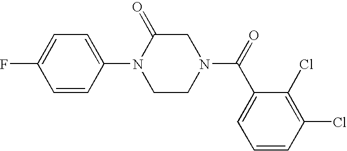

1-(4-Fluorophenyl)-2-piperazinone (150 mg, 0.77 mmol, prepared as described above for Example 12) and N-ethyl-N-(1-methylethyl)-2-propanamine (0.20 ml, 1.16 mmol) were added together in dichloromethane (4 ml) at 0° C. Subsequently 2,3-dichlorobenzoyl chloride (178 mg, 0.85 mmol) was added portionwise. The mixture was left under argon and in an icebath and allowed to return to room temperature whilst being stirred constantly overnight. Dichloromethane and aqueous 3N citric acid were then added and the mixture was extracted into dichloromethane (×2). The dichloromethane layers were combined and washed sequentially with water (×1), saturated aqueous sodium hydrogen carbonate (×1), water (×1), and brine (×1), and then dried over magnesium sulphate. The solvent was evaporated in vacuo to give 4-[(2,3-dichlorophenyl)carbonyl]-1-(4-fluorophenyl)-2-piperazinone (224 mg) as a white solid. LC/MS [M+H]⁺=367, retention time=2.62 minutes.

### Example 14

### 1-(3-Chlorophenyl)-4-[(2,3-dichlorophenyl)carbonyl]-2-piperazinone (E14)

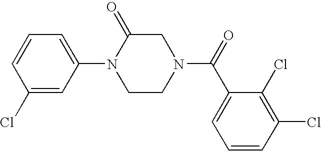

1-(3-Chlorophenyl)-4-[(2,3-dichlorophenyl)carbonyl]-2-piperazinone was prepared in a manner analogous to that described above for the synthesis of 4-[(2,3-Dichlorophenyl)carbonyl]-1-(4-fluorophenyl)-2-piperazinone in Example 13 but 1-(3-chlorophenyl)-2-piperazinone was used in place of the 1-(4-fluorophenyl)-2-piperazinone. LC/MS [M+H]⁺=385, retention time=2.81 minutes.

### Example 15

### 1-[(2-Chloro-4-fluorophenyl)methyl]-4-[(2,3-dichlorophenyl)carbonyl]-2-piperazinone (E15)

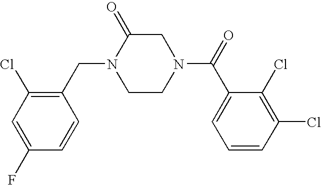

4-[(2,3-Dichlorophenyl)carbonyl]-2-piperazinone (200 mg, 0.73 mmol, prepared as described below) and sodium hydride (60% dispersion in mineral oil) (35.1 mg, 0.88 mmol) were added together in N,N-dimethylformamide (3 ml) at 0° C. After 15 minutes, 1-(bromomethyl)-2-chloro-4-fluorobenzene (327 mg, 1.47 mmol) was added. The mixture was left under argon and in an icebath and allowed to return to room temperature whilst being stirred constantly overnight. The reaction was quenched by the addition of methanol and saturated aqueous ammonium chloride and then the solvents were evaporated in vacuo. The residue was dissolved in dichloromethane and saturated aqueous sodium hydrogen carbonate, and then extracted into dichloromethane (×3). The combined organic extracts were washed with water (×1), then brine (×1), and dried with magnesium sulphate. The solvent was evaporated in vacuo and the crude product was purified by flash-silica gel chromatography, eluting with 30-70% ethyl acetate in isohexane, to give 1-[(2-chloro-4-fluorophenyl)methyl]-4-[(2,3-dichlorophenyl)carbonyl]-2-piperazinone (165 mg) as a clear/white solid. LC/MS [M+H]⁺=417, retention time=2.88 minutes.

The 4-[(2,3-dichlorophenyl)carbonyl]-2-piperazinone used in the above procedure was prepared as follows:

2-piperazinone (1 g, 9.99 mmol) and N-ethyl-N-(1-methylethyl)-2-propanamine (2.62 ml, 14.98 mmol) were added together in dichloromethane (20 ml) at 0° C. Subsequently 2,3-dichlorobenzoyl chloride (2.30 g, 10.99 mmol) was added portionwise. The mixture was left under argon and in an icebath and allowed to return to room temperature whilst being stirred constantly for 3 hours. Dichloromethane and saturated aqueous sodium hydrogen carbonate were added and the mixture was extracted into dichloromethane (×3). The dichloromethane layers were combined and washed with water (×1), and then brine (×1), and dried with magnesium sulphate. The solvent was evaporated in vacuo and the crude product was purified by flash-silica gel chromatography, eluting with 0-5% methanol in dichloromethane, to give 4-[(2,3-dichlorophenyl)carbonyl]-2-piperazinone (1.4 g) as a white solid. LC/MS [M+H]⁺=273, retention time=1.76 minutes.

### Examples 16-18 (E16-E18)

In a manner analogous to that described for Example 15 above the compounds tabulated below (Table 1) were prepared by substituting the appropriate alkyl bromide (or iodide) for the 1-(bromomethyl)-2-chloro-4-fluorobenzene used in the above procedure. All of the alkyl halides used in Table 1 are available from commercial sources.

| Example | | | | |
|---|---|---|---|---|
| Number | Structure & Name | Alkyl Halide | M + H | RT |
| E16 | 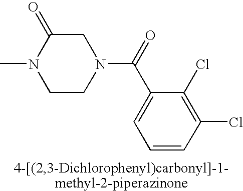 | Iodomethane | 287 | 1.92 |
| E17 | 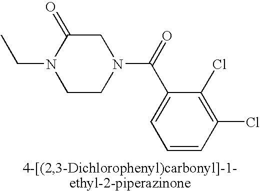 | Iodoethane | 301 | 2.10 |
| E18 | 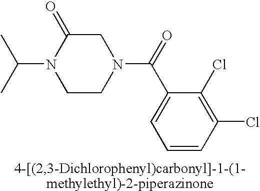 | 2-Bromo propane | 315 | 2.27 |

### Example 19

### 4-[(2,3-Dichlorophenyl)carbonyl]-1-(2,4-difluorophenyl)-2-piperazinone (E19)

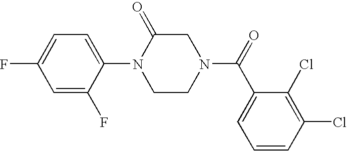

To 1-(2,4-difluorophenyl)-2-piperazinone (200 mg, 0.94 mmol, prepared as described below) in dichloromethane (3 ml) at 0° C. was added N-ethyl-N-(1-methylethyl)-2-propanamine (0.25 ml, 1.41 mmol), and then, portionwise, 2,3-dichlorobenzoyl chloride (217 mg, 1.04 mmol). The mixture was stirred at 0° C. for 30 minutes and then at room temperature overnight. Dichloromethane and aqueous 3N citric acid were added and the mixture was extracted into dichloromethane (×2). The dichloromethane layers were combined and washed sequentially with water (×1), saturated aqueous sodium hydrogen carbonate (×1), brine (×1), and then dried over magnesium sulphate. The solvent was evaporated in vacuo and the crude product purified further by column chromatography on flash-silica gel, eluting with 0-100% ethyl acetate in iso-hexane. Relevant fractions were combined and the solvent was evaporated in vacuo. The crude product was purified further by mass-directed automated HPLC to give 4-[(2,3-dichlorophenyl)carbonyl]-1-(2,4-difluorophenyl)-2-piperazinone (99 mg) as a white solid.

LC/MS [M+H]⁺=385, retention time=2.65 minutes.

The 1-(2,4-difluorophenyl)-2-piperazinone used in the above procedure was prepared as follows:

A suspension of 2-piperazinone (2 g, 20.0 mmol), 2,4-difluoro-1-iodobenzene (7.2 ml, 60.0 mmol), copper(I) iodide (0.761 g, 4.0 mmol), N,N,N′,N′-tetramethyl-1,2-ethanediamine (1.21 ml, 8.0 mmol) and potassium phosphate (12.7 g, 60.0 mmol) in 1,4-dioxane (30 ml) was heated at reflux (110° C.) under argon overnight. Further 2,4-difluoro-1-iodobenzene (1.5 eq.), copper(I) iodide (0.1 eq.) and N,N,N′,N′-tetramethyl-1,2-ethanediamine (0.2 eq.) were added and the mixture left for a further 24 hours. The mixture was cooled to room temperature, and then diluted with methanol and filtered through celite, washing with methanol. The filtrate was evaporated in vacuo and the residue was dissolved in dichloromethane and a solution of 0.88 aqueous ammonia in water (20%). The resulting mixture was extracted into dichloromethane (×3), and then the combined organic extracts were washed with water (×2) and then dried over magnesium sulphate. The solvent was evaporated in vacuo and the residue was dissolved in methanol and then purified further by SCX, eluting first with methanol and then with 2M ammonia in methanol. The basic fractions were combined and the solvent was evaporated in vacuo. The crude product was purified further by column chromatography on flash-silica gel, eluting with 0-12% methanol in dichloromethane. The relevant fractions were combined and the solvent evaporated in vacuo to give a brown oil which was further purified by column chromatography, eluting with 50-100% ethyl actetate in isohexane, and then 0-50% methanol in ethyl acetate to give 1-(2,4-difluorophenyl)-2-piperazinone (600 mg) as a dark brown gum, which was used without further purification. LC/MS [M+H]⁺=213, retention time=0.71 minutes.

### Example 20

### 4-[(2,4-Dichlorophenyl)carbonyl]-1-(2,4-difluorophenyl)-2-piperazinone (E20)

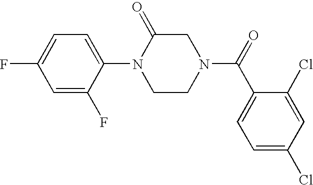

To 1-(2,4-difluorophenyl)-2-piperazinone (200 mg, 0.94 mmol, prepared as described above for Example 19) in dichloromethane (3 ml) at 0° C. was added N-ethyl-N-(1-methylethyl)-2-propanamine (0.25 ml, 1.41 mmol), and then, portionwise, 2,4-dichlorobenzoyl chloride (217 mg, 1.04 mmol). The reaction was stirred at 0° C. for 30 minutes and then at room temperature overnight.

Saturated aqueous sodium hydrogen carbonate was then added and the mixture was extracted into dichloromethane (×2). The combined organic extracts were washed with water (×1) and then dried over magnesium sulphate. The solvent was evaporated in vacuo and the crude product was purified further by column chromatography on flash-silica gel, eluting with 0-60% ethyl acetate in iso-hexane. The relevant fractions were combined and the solvent evaporated in vacuo. The resulting residue was purified again by mass-directed automated HPLC to give 4-[(2,4-dichlorophenyl)carbonyl]-1-(2,4-difluorophenyl)-2-piperazinone (160 mg) as a white solid. LC/MS [M+H]⁺=385, retention time=2.70 minutes.

### Example 21

### 4-{[2-chloro-3-(trifluoromethyl)phenyl]carbonyl}-1-(2,4-difluorophenyl)-2-piperazinone (E21)

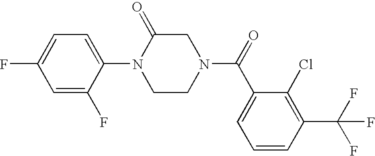

To a stirred solution of 1-(2,4-difluorophenyl)-2-piperazinone (200 mg, 0.943 mmol, prepared as described above for Example 19), DMAP (4-dimethylaminopyridine, 461 mg, 3.77 mmol) and 1-(3-dimethylaminopropyl)-3-ethylcarbodiimide hydrochloride (289 mg, 1.508 mmol) in dichloromethane (DCM) (3 ml) was added 2-chloro-3-(trifluoromethyl)benzoic acid (233 mg, 1.037 mmol), and the reaction was stirred at RT overnight. The reaction mixture was diluted with DCM and 3N Citric Acid (aq.), and the product was extracted into DCM (×2). The combined organic extracts were washed with water (×1), NaHCO₃ (sat., aq.), brine (×1) and then dried (MgSO₄). The solvent was evaporated in vacuo to give a dark brown oil, 360 mg, which was purified by MDAP. The relevant fractions were combined and the solvent evaporated in vacuo to give a yellow oil, which by TLC (50% EtOAc/iso-Hexane) contained a baseline impurity. This was purified further by column chromatography on silica gel, eluting with 0-100% EtOAc/iso-Hexane. The relevant fractions were combined and the solvent evaporated in vacuo to give a pale yellow foam. LCMS and NMR shows this to contain impurities, so this was purified again by MDAP. The relevant fractions were combined and the solvent evaporated in vacuo to give a white solid, 4-{[2-chloro-3-(trifluoromethyl)phenyl]carbonyl}-1-(2,4-difluorophenyl)-2-piperazinone (95 mg, 0.216 mmol, 22.87% yield), LC/MS [M+H]⁺=419, retention time=2.76 minutes.

### Example 22

### 4-[(2,3-dichlorophenyl)carbonyl]-1-(1-naphthalenyl)-2-piperazinone (E22)

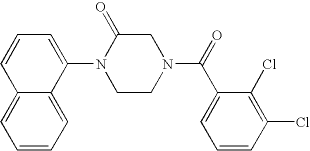

In a round-bottomed flask was added 1-(1-naphthalenyl)-2-piperazinone (100 mg, 0.442 mmol) and DIPEA (0.116 ml, 0.663 mmol) in Dichloromethane (DCM) (4 ml) to give a colorless solution. The reagents were cooled in an ice bath and 2,3-dichlorobenzoyl chloride (102 mg, 0.486 mmol) was slowly added under an argon atmosphere. Once the 2,3-dichlorobenzoyl chloride had been added the reaction was stirred for one hour at 0° C. and then at ambient temperature for 2 hours. Dichloromethane and aqueous 3N citric acid were added and the product was extracted into dichloromethane. The dichloromethane layer was washed with water, saturated aqueous sodium hydrogen carbonate, water and brine and then dried over magnesium sulphate. The solvent was evaporated in vacuo. The product was triturated with methanol and then dried on the high vacuum and placed in the oven overnight to give 4-[(2,3-dichlorophenyl)carbonyl]-1-(1-naphthalenyl)-2-piperazinone (110 mg, 0.262 mmol, 59.2% yield) as a pale orange solid. LC/MS [M+H]⁺=399, retention time=2.89 minutes.

The 1-(1-naphthalenyl)-2-piperazinone used in the above reaction was prepared in a manner analogous to that described in Example 4 for the preparation of 1-(2-methylphenyl)-2-piperazinone but using 1-naphthylamine in the place of o-toluidine.

### Example 23

### 4-[(2-chloro-4-fluorophenyl)carbonyl]-1-(1-naphthalenyl)-2-piperazinone (E23)

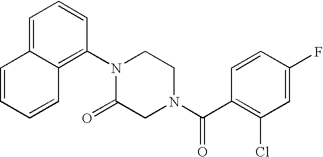

Thionyl chloride (0.016 mL, 0.22 mmol) in dichloromethane (0.5 mL) was added to a solution of 2-chloro-4-fluorobenzoic acid (0.036 g, 0.21 mmol) and pyridine (0.043 mL, 0.42 mmol) in dichloromethane (1.5 mL) and stirred under argon for 30 minutes at room temperature. 1-(1-Naphthalenyl)-2-piperazinone hydrochloride (0.070 g, 0.21 mmol) and pyridine (0.043 mL, 0.42 mmol) in dichloromethane (2 mL) was then added (solution turned clear yellow) and the reaction stirred at room temperature under argon for 2 hrs. Reaction washed with 2N HCl (10 mL) and saturated sodium hydrogen carbonate (10 mL), separated by hydrophobic frit, and reduced under vacuum to leave an off-white solid. This was purified by MDAP and freeze-dried to leave a white solid. Some remaining impurities were removed by further MDAP and freeze drying then gave pure 4-[(2-chloro-4-fluorophenyl)carbonyl]-1-(1-naphthalenyl)-2-piperazinone (0.012 g) as a white solid. LC/MS [M+H]⁺=382.93, retention time=2.71 minutes.

The 1-(1-Naphthalenyl)-2-piperazinone hydrochloride used in the above synthesis was prepared in the following manner:

i) Triethylamine (3.4 mL, 24.54 mmol) was added to a suspension of N-(1-naphthyl)ethylenediamine dihydrochloride (2.12 g, 8.18 mmol) in tetrahydrofuran (60 mL). Benzylchloroformate (1.16 mL, 8.18 mmol) was added slowly and the reaction stirred at room temperature for 3 hrs. Reaction stirred at room temperature for a further 3 hrs and then overnight. Benzyl chloroformate (0.12 mL, 0.82 mmol) was added again and the reaction stirred for 6 hrs. Solvent removed under vacuum and partitioned between DCM (100 mL) and water (100 mL). Aqueous layer extracted with IPA/CHCl₃ (3:1) (100 mL) and combined organics washed with saturated sodium hydrogen carbonate solution (50 mL), then 2N HCl (50 mL), and separated by hydrophobic frit, then concentrated under vacuum. Purification by automated silica gel flash column chromatography, eluting with a 0-40% gradient of ethyl acetate in hexane over 40 minutes, gave phenylmethyl [2-(1-naphthalenylamino)ethyl]-carbamate (0.888 g) as a clear oil.
ii) Sodium hydroxide (1.7 mL, 3.43 mmol) was added to a solution of phenylmethyl [2-(1-naphthalenylamino)ethyl]carbamate (0.88 g, 2.75 mmol) in DCM (10 mL) and stirred vigorously for 5 minutes, then bromoacetyl bromide (0.263 mL, 3.02 mmol) was added and the reaction stirred at room temperature for 3 hrs. Reaction washed with water and organic layer separated by hydrophobic frit. Aqueous layer extracted with DCM (10 mL) and combined organics concentrated under vacuum to leave phenylmethyl {2-[(bromoacetyl)(1-naphthalenyl)amino]ethyl}carbamate (1.29 g) as a clear oil which was used in the next step without further purification.
iii) Potassium carbonate (1.14 g, 8.25 mmol) was added to a solution of phenylmethyl {2-[(bromoacetyl)(1-naphthalenyl)amino]ethyl}carbamate (1.29 g, 2.75 mmol) in DMF (15 mL) and stirred overnight at room temperature. Reaction then stirred at 50° C. overnight. Reaction reduced to dryness under vacuum and partitioned between DCM (50 mL) and water (50 mL). Aqueous layer extracted with DCM (30 mL) and separated by hydrophobic frit. Organic layers reduced under vacuum and purified by automated silica-gel flash column chromatography, eluting with a gradient of 10%-100% of ethyl acetate in hexane, to give partially pure phenylmethyl 4-(1-naphthalenyl)-3-oxo-1-piperazinecarboxylate which was used in the next step.
iv) Partially pure phenylmethyl 4-(1-naphthalenyl)-3-oxo-1-piperazinecarboxylate (0.500 g, ˜0.78 mmol) was dissolved in ethanol (50 mL) and acetic acid (5 mL) and treated with palladium on carbon (10%, 0.100 g) then stirred under hydrogen at room temperature overnight. Reaction filtered through kieselguhr (washing with ethanol) then reduced under vacuum to leave a yellow gum. Dissolved in minimum of DCM and treated with 1M HCl in ethanol (2 mL). The resulting yellow solid was filtered off and dried to give 1-(1-Naphthalenyl)-2-piperazinone hydrochloride (0.144 g).

### Example 24

### 4-{[2-chloro-3-(trifluoromethyl)phenyl]carbonyl}-1-(1-naphthalenyl)-2-piperazinone (E24)

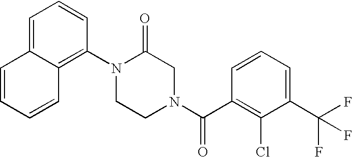

A solution of 1-(1-naphthalenyl)-2-piperazinone (112 mg, 0.495 mmol, prepared in a manner analogous to that described in Example 22), EDC (152 mg, 0.792 mmol) and DMAP (242 mg, 1.980 mmol) in Dichloromethane (DCM) (4 ml) was stirred at room temperature under an argon atmosphere. 2-chloro-3-(trifluoromethyl)benzoic acid (111 mg, 0.495 mmol) was added portionwise and leave to stir overnight. Dichloromethane and aqueous 3N citric acid were added and the product was extracted into dichloromethane (×2). The dichloromethane layer was washed with water (×1), saturated aqueous sodium hydrogen carbonate (×1), water (×1), and brine (×1) then it was dried on magnesium sulfate and evaporated in vacuo.

Product was purified by MDAP and fractions combined and then the solvent was evaporated in vacuo to give a white solid 4-{[2-chloro-3-(trifluoromethyl)phenyl]carbonyl}-1-(1-naphthalenyl)-2-piperazinone (75 mg, 0.173 mmol, 35.0% yield). LC/MS [M+H]⁺=432.9, retention time=2.97 minutes.

### Example 25

### 4-{[2-chloro-3-(trifluoromethyl)phenyl]carbonyl}-1-[2-(1-methylethyl)phenyl]-2-piperazinone (E25)

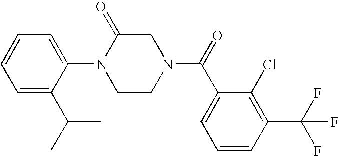

A solution of 1-[2-(1-methylethyl)phenyl]-2-piperazinone (100 mg, 0.458 mmol), EDC (141 mg, 0.733 mmol) and DMAP (224 mg, 1.832 mmol) in dichloromethane (DCM) (4 mL) was stirred at room temperature under an argon atmosphere. 2-chloro-3-(trifluoromethyl)benzoic acid (103 mg, 0.458 mmol) was added portionwise and leave to stir overnight. Dichloromethane and aqueous 3N citric acid were added and the product was extracted into dichloromethane (×2). The dichloromethane layer was washed with water (×1), saturated aqueous sodium hydrogen carbonate (×1), water (×1), and brine (×1) then it was dried on magnesium sulfate and evaporated in vacuo. Product was purified by MDAP and fractions combined and then the solvent was evaporated in vacuo to give a white solid 4-{[2-chloro-3-(trifluoromethyl)phenyl]carbonyl}-1-[2-(1-methylethyl)phenyl]-2-piperazinone (35 mg, 0.082 mmol, 17.98% yield).

LC/MS [M+H]⁺=425, retention time=3.05 minutes.

The 1-[2-(1-methylethyl)phenyl]-2-piperazinone used in the above reaction was prepared in a manner analogous to that described in Example 4 for the preparation of 1-(2-methylphenyl)-2-piperazinone but using [2-(1-methylethyl)phenyl]amine in the place of o-toluidine.

### Example 26

### 4-[(2-chloro-4-fluorophenyl)carbonyl]-1-[2-(1-methylethyl)phenyl]-2-piperazinone (E26)

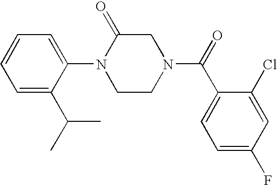

4-[(2-chloro-4-fluorophenyl)carbonyl]-1-[2-(1-methylethyl)phenyl]-2-piperazinone was prepared in a manner analogous to that described above for the synthesis 4-{[2-chloro-3-(trifluoromethyl)phenyl]carbonyl}-1-[2-(1-methylethyl)phenyl]-2-piperazinone in Example 25 but 2-chloro-4-fluoro benzoic acid was used in place of the 2-chloro-3-(trifluoromethyl)benzoic acid. LC/MS [M+H]⁺=375.0, retention time=2.82 minutes.

### Example 27

### 4-[(2,3-dichlorophenyl)carbonyl]-1-[2-(1-methylethyl)phenyl]-2-piperazinone (E27)

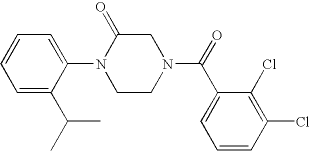

In a round-bottomed flask was added 1-[2-(1-methylethyl)phenyl]-2-piperazinone (100 mg, 0.458 mmol, prepared in an analogous manner to that described in Example 25) and DIPEA (0.120 ml, 0.687 mmol) in dichloromethane (DCM) (4 ml) to give a colorless solution. The reagents were cooled in an ice bath and then 2,3-dichlorobenzoyl chloride (106 mg, 0.504 mmol) was slowly added under an argon atmosphere. The reaction was left to stir overnight. Dichloromethane and aqueous 3N citric acid were added and the product was extracted into dichloromethane. Dichloromethane layer was washed with water, saturated aqueous sodium hydrogen carbonate, water and brine and then dried over magnesium sulfate. Solvent was evaporated in vacuo. The product was purified by column chromatography on silica gel eluting with a gradient of 0% to 50% ethyl acetate in iso-hexane. Fractions were collected and solvent was evaporated in vacuo. The product was purified again by MDAP and fractions combined and then the solvent was evaporated in vacuo to give a white solid 4-[(2,3-dichlorophenyl)carbonyl]-1-[2-(1-methylethyl)phenyl]-2-piperazinone (40 mg, 0.102 mmol, 22.32% yield). LC/MS [M+H]⁺=390.9, retention time=2.95 minutes.

### Example 28

### 4-[(2,3-dichlorophenyl)carbonyl]-1-(4-fluoro-2-methyl phenyl)-2-piperazinone (E28)

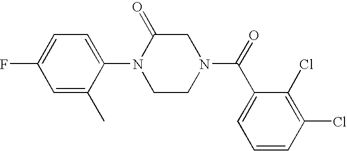

In a round-bottomed flask was added 1-(4-fluoro-2-methylphenyl)-2-piperazinone (90 mg, 0.432 mmol) and DIPEA (0.113 ml, 0.648 mmol) in dichloromethane (DCM) (4 ml) to give a colorless solution. The reagents were cooled in an ice bath and then 2,3-dichlorobenzoyl chloride (100 mg, 0.475 mmol) was slowly added under an argon atmosphere. The reaction was left to stir overnight. Dichloromethane and aqueous 3N citric acid were added and the product was extracted into dichloromethane. Dichloromethane layer was washed with water, saturated aqueous sodium hydrogen carbonate, water and brine and then dried over magnesium sulfate. Solvent was evaporated in vacuo. Product was purified by MDAP and fractions combined and then the solvent was evaporated in vacuo to give a white solid 4-[(2,3-dichlorophenyl)carbonyl]-1-(4-fluoro-2-methylphenyl)-2-piperazinone (73.5 mg, 0.193 mmol, 44.6% yield). LC/MS [M+H]⁺=380.9, retention time=2.72 minutes.

The 1-(4-fluoro-2-methylphenyl)-2-piperazinone used in the above synthesis was prepared in the following manner:

To a stirred mixture of 2-piperazinone (500 mg, 4.99 mmol), 4-fluoro-1-iodo-2-methylbenzene (1000 mg, 4.24 mmol) in 1,4-dioxane (15 ml) was added potassium phosphate (5301 mg, 24.97 mmol), copper (I) iodide (951 mg, 4.99 mmol) and trans-N,N-dimethylcyclohexane-1,2-diamine (0.787 ml, 4.99 mmol) and the mixture was heated at reflux under argon for 3 hours. The mixture was cooled to room temperature and then diluted with MeOH. Reaction mixture was filtered through a pad of celite, washing with MeOH and then the filtrate evaporated in vacuo. Residue was dissolved in DCM and a solution of 0.88 aqueous NH₃ (˜10 ml) in water (˜100 ml), and product was extracted into DCM (×2). Combined organic extracts were washed with water (×1) and dried (MgSO₄). Solvent was evaporated in vacuo to give a dark brown oil (˜1 g). Crude product was purified by reverse phase column chromatography, eluting with 5-100% acetonitrile in water. Relevant fractions were passed through an SCX cartridge, eluting first with MeOH and then with 2M NH₃/MeOH. Basic fractions were combined and solvent evaporated in vacuo to give a dark yellow oil. This was purified further by column chromatography on silica gel, eluting with 0-20% MeOH/DCM. Relevant fractions were combined and solvent evaporated in vacuo to give a yellow gum, 1-(4-fluoro-2-methylphenyl)-2-piperazinone (110 mg, 0.528 mmol, 10.58% yield).

### Example 30

### 1-(2-chloro-4-fluorophenyl)-4-[(2-chloro-3-methylphenyl)carbonyl]-2-piperazinone (E30)

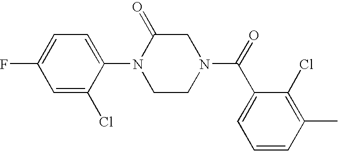

1-(2-chloro-4-fluorophenyl)-4-[(2-chloro-3-methylphenyl)carbonyl]-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but 2-chloro-3-methyl benzoic acid was used in place of the 2-chloro-3-(trifluoromethyl)benzoic acid. LC/MS [M+H]⁺=380.9, retention time=1.00 minutes. (2 minute method)

### Example 31

### 1-(2-chloro-4-fluorophenyl)-4-[(2,6-dichlorophenyl)carbonyl]-2-piperazinone (E31)

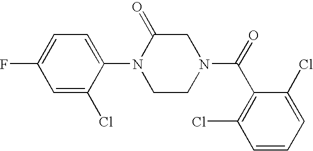

1-(2-chloro-4-fluorophenyl)-4-[(2,6-dichlorophenyl)carbonyl]-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 2,6-dichlorobenzoyl chloride was used in place of the 2,3-dichlorobenzoyl chloride. LC/MS [M+H]⁺=402.9, retention time=2.64 minutes.

### Example 32

### 4-[(2-chloro-3,4-difluorophenyl)carbonyl]-1-(2-chloro-4-fluorophenyl)-2-piperazinone (E32)

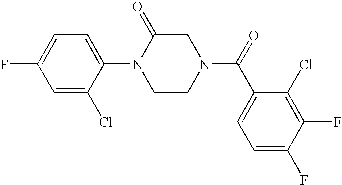

4-[(2-chloro-3,4-difluorophenyl)carbonyl]-1-(2-chloro-4-fluorophenyl)-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but 2-chloro-3,4-difluorobenzoic acid was used in place of the 2-chloro-3-(trifluoromethyl)benzoic acid. LC/MS [M+H]⁺=402.8, retention time=2.72 minutes.

### Example 33

### 4-[(2-chloro-3,6-difluorophenyl)carbonyl]-1-(2-chloro-4-fluorophenyl)-2-piperazinone (E33)

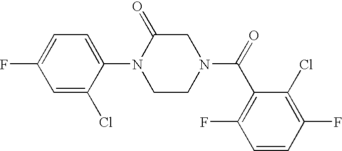

4-[(2-chloro-3,6-difluorophenyl)carbonyl]-1-(2-chloro-4-fluorophenyl)-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but 2-chloro-3,6-difluorobenzoic acid was used in place of the 2-chloro-3-(trifluoromethyl)benzoic acid. LC/MS [M+H]⁺=402.9, retention time=2.67 minutes.

### Example 35

### 4-{[2-chloro-3-(trifluoromethyl)phenyl]carbonyl}-1-methyl-2-piperazinon (E35)

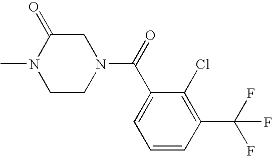

4-{[2-chloro-3-(trifluoromethyl)phenyl]carbonyl}-1-methyl-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but 2-chloro-3-(trifluoromethyl)benzoic acid and 1-methyl-2-piperazinone were used in place of the 2-chloro-3-(trifluoromethyl)benzoic acid and 1-(2-chloro-4-fluorophenyl)-2-piperazinone respectively. LC/MS [M+H]⁺=321.1, retention time=2.21 minutes.

### Example 36

### 4-[(2,4-dichlorophenyl)carbonyl]-1-methyl-2-piperazinone (E36)

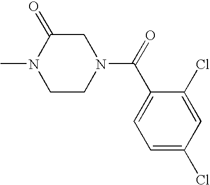

4-[(2,4-dichlorophenyl)carbonyl]-1-methyl-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 2,4-dichlorobenzoyl chloride and 1-methyl-2-piperazinone were used in place of the 2,3-dichlorobenzoyl chloride and 1-(2-chloro-4-fluorophenyl)-2-piperazinone respectively. LC/MS [M+H]⁺=287.1, retention time=2.07 minutes.

### Example 37

### 4-[(2-chloro-4-fluorophenyl)carbonyl]-1-methyl-2-piperazinone (E37)

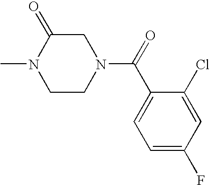

4-[(2-chloro-4-fluorophenyl)carbonyl]-1-methyl-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 2-chloro-4-fluorobenzoyl chloride and 1-methyl-2-piperazinone were used in place of the 2,3-dichlorobenzoyl chloride and 1-(2-chloro-4-fluorophenyl)-2-piperazinone respectively. LC/MS [M+H]⁺=271.1, retention time=1.81 minutes.

### Example 38

### 2-{-4-[(2,3-dichlorophenyl)carbonyl]-2-oxo-1-piperazinyl}-5-fluorobenzonitrile (E38)

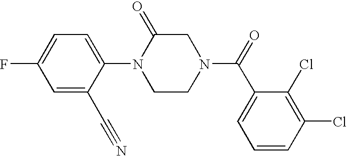

To a suspension of 5-fluoro-2-(2-oxo-1-piperazinyl)benzonitrile.HCl (90 mg, 0.352 mmol) in dichloromethane (DCM) (7 ml) at 0° C. under argon was added the DIPEA (0.154 ml, 0.880 mmol) and then, portionwise, the 2,3-dichlorobenzoyl chloride (81 mg, 0.387 mmol). The reaction mixture was allowed to warm to RT and left to stir at RT under argon overnight.

Dichloromethane (15 ml) and aqueous 3N citric acid (15 ml) were added and the product extracted into dichloromethane (×2). The dichloromethane layers were combined and washed sequentially with water (15 ml) (×1), saturated aqueous sodium hydrogen carbonate (15 ml) (×1), water (15 ml) (×1), and brine (15 ml) (×1), and the dried over magnesium sulphate. The solvent was evaporated in vacuo to give a brown oil.

The crude product was purified by flash-silica gel chromatography, eluting with 0-10% MeOH/EtOAc. Product-containing fractions were concentrated under vacuum to give an orange solid, 2-{-4-[(2,3-dichlorophenyl)carbonyl]-2-oxo-1-piperazinyl}-5-fluorobenzonitrile (45 mg, 0.109 mmol, 31.0% yield), LC/MS [M+H]⁺=392.0, retention time=2.28 minutes.

The 5-fluoro-2-(2-oxo-1-piperazinyl)benzonitrile.HCl used as the starting material in the above synthesis was prepared in the following manner:

i) To methyl N-{[(1,1-dimethylethyl)oxy]carbonyl}glycinate (15.63 ml, 106 mmol) in N,N-dimethylformamide (DMF) (100 ml) at 0° C. under argon was added, portionwise, the sodium hydride (4.44 g, 111 mmol) and the reaction mixture stirred for 10 minutes before adding the allyl bromide (10.06 ml, 116 mmol) portionwise. The reaction mixture was left to stir at 0° C. under argon for 1 hour and then allowed to warm to RT and stirred at RT overnight. The reaction mixture was cooled to 0° C. and then quenched by the portionwise addition of NH₄Cl (sat., aq.) (˜100 ml) and ethyl acetate (˜120 ml). The resulting mixture was extracted into ethyl acetate (×3) and then the combined organic layers were washed with brine and dried over magnesium sulphate. The solvent was evaporated in vacuo to give a yellow oil.

The crude product was purified further by flash-silica gel chromatography, eluting with 0-50% Et₂O/iso-hexane. The fractions containing product were combined and the solvent evaporated in vacuo to give a pale yellow oil, methyl N-{[(1,1-dimethylethyl)oxy]carbonyl}-N-2-propen-1-ylglycinate (15.1 g, 65.9 mmol, 62.3% yield).

ii) A solution of methyl N-{[(1,1-dimethylethyl)oxy]carbonyl}-N-2-propen-1-ylglycinate (15.0 g, 65.4 mmol) in dichloromethane (DCM) (300 ml) at −70° C. was flushed with argon for 10 mins and then with ozone for ˜3 hours (until solution turned blue). The excess ozone was purged with argon, and then the dimethyl sulfide (14.52 ml, 196 mmol) was added and the reaction mixture was allowed to warm to RT and stirred at RT under argon overnight.

The solvent was evaporated in vacuo to give a colourless oil, which was taken up in EtOAc and washed with water (×2). The EtOAc layer was evaporated in vacuo to give a colourless oil, methyl N-{[(1,1-dimethylethyl)oxy]carbonyl}-N-(2-oxoethyl)glycinate (17.2 g, 37.2 mmol, 56.8% yield).

iii) To methyl N-{[(1,1-dimethylethyl)oxy]carbonyl}-N-(2-oxoethyl)glycinate (1 g, 2.162 mmol) in dichloromethane (DCM) (15 ml) at 0° C. was added, in sequence, the Molecular Sieves (4 Å) (500 mg, 2.162 mmol), the 2-amino-5-fluorobenzonitrile (0.294 g, 2.162 mmol) and the acetic acid (3 drops) (catalytic). The reaction mixture was stirred at 0° C. for 15 minutes and then the sodium triacetoxyborohydride (0.550 g, 2.59 mmol) was added portionwise. The reaction mixture was allowed to warm to RT and stirred at RT under argon over the weekend.

The reaction mixture was diluted with DCM and then filtered. The DCM filtrate was quenched by the addition of NaHCO₃ (sat., aq.) (˜25 ml) and then the product was extracted into DCM (×2). The combined organic extracts were dried over magnesium sulphate and then the solvent evaporated in vacuo to give a yellow oil.

The crude product was purified further by flash-silica gel chromatography, eluting with 0-40% EtOAc/iso-hexane. Fractions containing product were combined and the solvent evaporated in vacuo to give methyl N-{2-[(2-cyano-4-fluorophenyl)amino]ethyl}-N-{[(1,1-dimethylethyl)oxy]carbonyl}glycinate (240 mg, 0.683 mmol, 31.6% yield).

iv) To methyl N-{2-[(2-cyano-4-fluorophenyl)amino]ethyl}-N-{[(1,1-dimethylethyl)oxy]carbonyl}glycinate (240 mg, 0.683 mmol) in 1,4-dioxane (2.5 ml) was added the hydrochloric acid (conc.) (0.4 ml, 13.16 mmol) and the reaction stirred at RT for 10 minutes and then heated at reflux for 2 hours.

The reaction mixture was cooled to RT and then the solvent was evaporated in vacuo to give a dark brown foam, 5-fluoro-2-(2-oxo-1-piperazinyl)benzonitrile.HCl (180 mg, 0.704 mmol, 103% yield).

### Example 39

### 4-[(2,3-dichlorophenyl)carbonyl]-1-(2-methylpropyl)-2-piperazinone (E39)

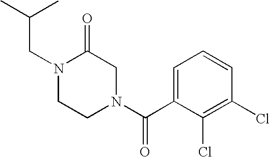

4-[(2,3-Dichlorophenyl)carbonyl]-1-(2-methylpropyl)-2-piperazinone was prepared in a manner analogous to that described above for the synthesis of 1-[(2-chloro-4-fluorophenyl)methyl]-4-[(2,3-dichlorophenyl)carbonyl]-2-piperazinone in Example 15 but 1-bromo-2-methylpropane was used in place of the 1-(bromomethyl)-2-chloro-4-fluorobenzene.

LC/MS=329/331 (M+H)⁺, retention time=2.44 (5 minute).

### Example 40

### 1-(2-chloro-4-fluorophenyl)-4-[(2-chloro-4-fluorophenyl)carbonyl]-2-piperazinone (E40)

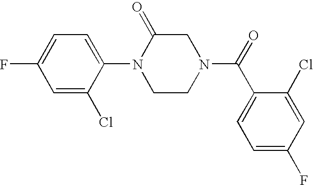

1-(2-Chloro-4-fluorophenyl)-4-[(2-chloro-4-fluorophenyl)carbonyl]-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 2-chloro-4-fluorobenzoyl chloride was used in place of the 2,3-dichlorobenzoyl chloride. LC/MS [M+H]⁺=384.8, retention time=0.97 minutes (2 minute method).

### Example 41

### 4-[(2-chloro-3-methylphenyl)carbonyl]-1-methyl-2-piperazinone (E41)

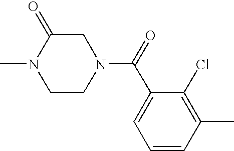

4-[(2-Chloro-3-methylphenyl)carbonyl]-1-methyl-2-piperazinone was prepared in a manner analogous to that described above for the synthesis of 1-(2-chloro-4-fluorophenyl)-4-{[2-chloro-3-(trifluoromethyl)phenyl]carbonyl}-2-piperazinone in Example 10 but 2-chloro-3-methylbenzoic acid and 1-methyl-2-piperazinone were used in place of the 2-chloro-3-(trifluoromethyl)benzoic acid and 1-(2-chloro-4-fluorophenyl)-2-piperazinone respectively. LC/MS [M+H]⁺=267.1, retention time=1.88 minutes.

### Example 42

### 4-{[2-chloro-3-(trifluoromethyl)phenyl]carbonyl}-1-ethyl-2-piperazinone (E42)

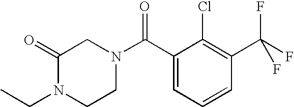

4-{[2-Chloro-3-(trifluoromethyl)phenyl]carbonyl}-1-ethyl-2-piperazinone was prepared in a manner analogous to that described above for the synthesis of 1-[(2-chloro-4-fluorophenyl)methyl]-4-[(2,3-dichlorophenyl)carbonyl]-2-piperazinone in Example 15 but iodoethane and 4-{[2-chloro-3-(trifluoromethyl)phenyl]carbonyl}-2-piperazinone were used in place of the 1-(bromomethyl)-2-chloro-4-fluorobenzene and 4-[(2,3-Dichlorophenyl)carbonyl]-2-piperazinone respectively.

LC/MS=335/337 (M+H)⁺, retention time=2.31 min.

The 4-{[2-chloro-3-(trifluoromethyl)phenyl]carbonyl}-2-piperazinone was in turn also prepared using the method described in Example 15 but using 2-chloro-3-(trifluoromethyl)benzoyl chloride instead of 2,3-dichlorobenzoyl chloride.

### Example 43

### 4-{[2-chloro-3-(trifluoromethyl)phenyl]carbonyl}-1-(1-methylethyl)-2-piperazinone (E43)

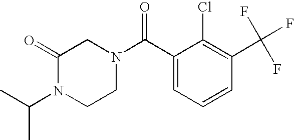

4-{[2-Chloro-3-(trifluoromethyl)phenyl]carbonyl}-1-(1-methylethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 2-bromopropane in place of the iodoethane.

LC/MS=349/351(M+H)⁺, retention time=2.47

### Example 44

### 4-{[2-chloro-3-(trifluoromethyl)phenyl]carbonyl}-1-(2-methylpropyl)-2-piperazinone (E44)

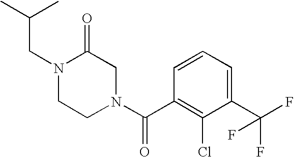

4-{[2-Chloro-3-(trifluoromethyl)phenyl]carbonyl}-1-(2-methylpropyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 1-iodo-2-methylpropane in place of the iodoethane.

LC/MS=363/365 (M+H)⁺, retention time=2.67.

### Example 45

### 1-(5-bromo-4-fluoro-2-methylphenyl)-4-{[2-chloro-3-(trifluoromethyl)phenyl]carbonyl}-2-piperazinone (E45)

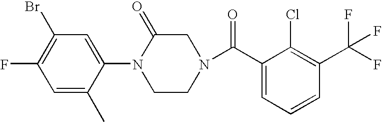

1-(5-Bromo-4-fluoro-2-methylphenyl)-4-{[2-chloro-3-(trifluoromethyl)phenyl]carbonyl}-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but using 1-(5-bromo-4-fluoro-2-methylphenyl)-2-piperazinone in place of 1-(2-chloro-4-fluorophenyl)-2-piperazinone. LC/MS [M+H]⁺=495, retention time=3.06 minutes.

The 1-(5-bromo-4-fluoro-2-methylphenyl)-2-piperazinone was in turn prepared using the method described in Example 4 for the preparation of 1-(2-methylphenyl)-2-piperazinone but using 5-bromo-4-fluoro-2-methylaniline in the place of o-toluidine.

### Example 46

### 1-(3-bromo-2-methyl phenyl)-4-{[2-chloro-3-(trifluoromethyl)phenyl]carbonyl}-2-piperazinone (E46)

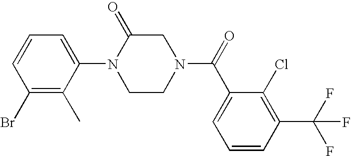

1-(3-Bromo-2-methylphenyl)-4-{[2-chloro-3-(trifluoromethyl)phenyl]carbonyl}-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 1-(3-bromo-2-methylphenyl)-2-piperazinone and 2-chloro-3-(trifluoromethyl)benzoyl chloride were used in place of 1-(2-chloro-4-fluorophenyl)-2-piperazinone and 2,3-dichlorobenzoyl chloride respectively. LC/MS [M+H]⁺=476.7, retention time=1.11 minutes (2 minute method).

The 1-(3-bromo-2-methylphenyl)-2-piperazinone was in turn prepared using the method described in Example 4 for the preparation of 1-(2-methylphenyl)-2-piperazinone but using 3-bromo-2-methylaniline in the place of o-toluidine.

### Example 47

### 4-{[2-chloro-3-(trifluoromethyl)phenyl]carbonyl}-1-(1-ethylpropyl)-2-piperazinone (E47)

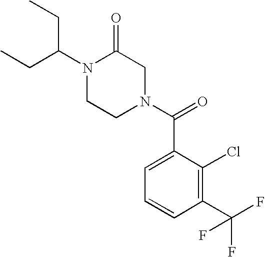

4-{[2-Chloro-3-(trifluoromethyl)phenyl]carbonyl}-1-(1-ethylpropyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 3-bromopentane in place of iodoethane.

LC/MS=377/379 (M+H)⁺, retention time=2.76 min.

### Example 48

### 4-{[2-chloro-3-(trifluoromethyl)phenyl]carbonyl}-1-(cyclopropyl methyl)-2-piperazinone (E48)

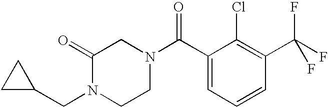

4-{[2-Chloro-3-(trifluoromethyl)phenyl]carbonyl}-1-(cyclopropylmethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using (bromomethyl)cyclopropane in place of iodoethane.

LC/MS=361/363 (M+H)⁺, retention time=2.55 min.

### Example 49

### 4-{[2-chloro-3-(trifluoromethyl)phenyl]carbonyl}-1-propyl-2-piperazinone (E49)

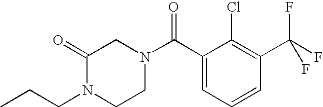

4-{[2-Chloro-3-(trifluoromethyl)phenyl]carbonyl}-1-propyl-2-piperazinone was prepared in a manner analogous to that described above for the synthesis of 4-{[2-chloro-3-(trifluoromethyl)phenyl]carbonyl}-1-ethyl-2-piperazinone in Example 42 but using 1-iodopropane in place of iodoethane.

LC/MS=349/351 (M+H)⁺, retention time=2.48 min.

### Example 50

### 2-(4-{[2-chloro-3-(trifluoromethyl)phenyl]carbonyl}-2-oxo-1-piperazinyl)-5-fluorobenzonitrile (E50)

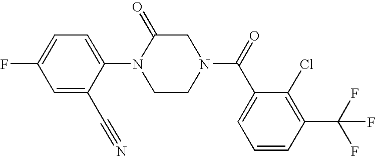

2-(4-{[2-Chloro-3-(trifluoromethyl)phenyl]carbonyl}-2-oxo-1-piperazinyl)-5-fluorobenzonitrile was prepared in a manner analogous to that described above for the synthesis of 1-(2-chloro-4-fluorophenyl)-4-[(2,3-dichlorophenyl)carbonyl]-2-piperazinone in Example 11 but 5-fluoro-2-(2-oxo-1-piperazinyl)benzonitrile.HCl (prepared as described in Example 38) and 2-chloro-3-(trifluoromethyl)benzoyl chloride were used in place of 1-(2-chloro-4-fluorophenyl)-2-piperazinone and 2,3-dichlorobenzoyl chloride respectively. [M+H]⁺=426.1, retention time=2.07 minutes.

### Example 51

### 4-{[2-chloro-3-(trifluoromethyl)phenyl]carbonyl}-1-(cyclobutyl methyl)-2-piperazinone (E51)

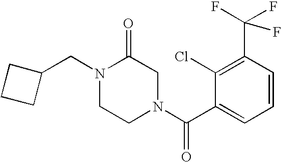

4-{[2-Chloro-3-(trifluoromethyl)phenyl]carbonyl}-1-(cyclobutylmethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using (bromomethyl)cyclobutane in place of iodoethane.

LC/MS=375/377 (M+H)⁺, retention time=2.49 min.

### Example 52

### 4-{[2-chloro-3-(trifluoromethyl)phenyl]carbonyl}-1-cyclopentyl-2-piperazinone (E52)

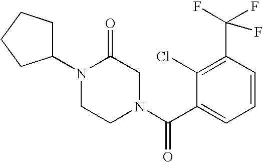

4-{[2-Chloro-3-(trifluoromethyl)phenyl]carbonyl}-1-cyclopentyl-2-piperazinone was prepared in a manner analogous to that described above for the synthesis of 4-{[2-chloro-3-(trifluoromethyl)phenyl]carbonyl}-1-ethyl-2-piperazinone in Example 42 but using bromocyclopentane in place of iodoethane.

LC/MS=375/377 (M+H)⁺, retention time=2.44 min.

### Example 53

### 1-(5-bromo-2-methylphenyl)-4-{[2-chloro-3-(trifluoromethyl)phenyl]carbonyl}-2-piperazinone (E53)

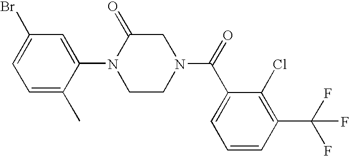

To 1-(5-bromo-2-methylphenyl)-2-piperazinone (260 mg, 0.966 mmol) in dichloromethane (DCM) (5 ml) was added first the polymer-bound triethylamine (453 mg, 1.449 mmol) and then the 2-chloro-3-(trifluoromethyl)benzoyl chloride (258 mg, 1.063 mmol), and the reaction mixture stirred at RT for 3 hours. NaHCO₃ (sat., aq.) (˜5 ml) was added and then the solution filtered through a phase separator cartridge, washing with DCM. The DCM filtrate was evaporated in vacuo to give a yellow oil. The crude product was purified by flash-silica gel column chromatography, eluting with 0-100% EtOAc/iso-hexane. Relevant fractions were combined and solvent evaporated in vacuo to give a pale yellow gum which was triturated with iso-hexane and the solid filtered off to give a white solid, 1-(5-bromo-2-methylphenyl)-4-{[2-chloro-3-(trifluoromethyl)phenyl]carbonyl}-2-piperazinone (300 mg, 0.599 mmol, 62.0% yield).

[M+H]⁺ 477, retention time 2.85 minutes.

The 1-(5-bromo-2-methylphenyl)-2-piperazinone used in the above synthesis was prepared in the following manner:

i) 5-Bromo-2-methylaniline (6 g, 32.2 mmol) in Tetrahydrofuran (THF) (60 ml) was cooled to ˜5° C. in an ice/water bath, and then a solution of potassium carbonate (12.26 g, 89 mmol) in water (30 ml) was added. The chloroacetyl chloride (3.23 ml, 40.3 mmol) was then added dropwise over 15 minutes to the rapidly stirred bi-phasic solution. The reaction was allowed to warm to RT while stirring for 1 hour (LCMS: N6089-39-R1), and then the organic layer was separated. The organic layer was cooled to ˜5° C. again and then the ethanolamine (7 ml, 116 mmol) was added. The reaction was warmed to RT and stirred at RT overnight. The reaction mixture was heated to 50° C. and stirred at 50° C. for 4 hours. After cooling to RT, EtOAc (40 ml) and water (20 ml) were added. Product appeared to crash out in EtOAc layer, so organic layer was separated and then filtered and dried, to give the product as a white solid, N1-(5-bromo-2-methylphenyl)-N2-(2-hydroxyethyl)glycinamide (8.05 g, 28.0 mmol, 87% yield).

M+H 287, 289; retention time 0.91 mins.

ii) To a part-suspension of N1-(5-bromo-2-methylphenyl)-N2-(2-hydroxyethyl)glycinamide (7.05 g, 24.55 mmol) in dichloromethane (DCM) (150 ml) and Tetrahydrofuran (THF) (150 ml) at 0° C. was added the BOC-Anhydride (6.27 ml, 27.0 mmol) and then the triethylamine (5 ml, 35.9 mmol). The reaction mixture was allowed to warm to RT and stirred at RT overnight. The solvent was removed under vacuum, and then ether was added. The resulting solid was filtered off and the filtrate was evaporated in vacuo to give a colourless oil, 1,1-dimethylethyl {2-[(5-bromo-2-methylphenyl)amino]-2-oxoethyl}(2-hydroxyethyl)carbamate (9.5 g, 24.53 mmol, 100% yield).
iii) Methanesulfonyl chloride (0.211 ml, 2.71 mmol) was added to a stirred solution of 1,1-dimethylethyl {2-[(5-bromo-2-methylphenyl)amino]-2-oxoethyl}(2-hydroxyethyl)carbamate (1 g, 2.58 mmol) in dichloromethane (DCM) (10 ml) and triethylamine (0.396 ml, 2.84 mmol). The reaction mixture was stirred at RT for 1 hour, and then a further 0.2 ml of triethylamine and 0.1 ml of MsCl were added and the reaction stirred at RT overnight. DCM and NaHCO₃ (sat., aq.) were added, and the product extracted into DCM (×2) and then the combined organic layers were dried over magnesium sulphate. The solvent was evaporated in vacuo to give a pale orange oil. The crude product was purified by column chromatography on silica gel, eluting with 50% EtOAc/iso-hexane. Relevant fractions were combined and solvent evaporated in vacuo to give a colourless oil, 2-({2-[(5-bromo-2-methylphenyl)amino]-2-oxoethyl}{[(1,1-dimethylethyl)oxy]carbonyl}amino)ethyl methanesulfonate (450 mg, 0.967 mmol, 37.4% yield).
iv) To 2-({2-[(5-bromo-2-methylphenyl)amino]-2-oxoethyl}{[(1,1-dimethylethyl)oxy]carbonyl}amino)ethyl methanesulfonate (5.3 g, 11.39 mmol) in N,N-dimethylformamide (DMF) (45 ml) was added portionwise the sodium hydride (60% dispersion) (500 mg, 12.50 mmol). The reaction mixture was stirred at RT under argon overnight. MeOH (3 ml) was added and then reaction mixture stirred at RT for 10 minutes and then solvent evaporated in vacuo. The residue was dissolved in DCM (20 ml) and NaHCO₃ (sat., aq.) (20 ml) and the product extracted into DCM (×2). The combined organic extracts were dried over magnesium sulphate and then solvent evaporated in vacuo and the residue triturated with Et₂₀ to give a white solid, 1,1-dimethylethyl 4-(5-bromo-2-methylphenyl)-3-oxo-1-piperazinecarboxylate (1.42 g, 3.85 mmol, 33.8% yield).
v) To 1,1-dimethylethyl 4-(5-bromo-2-methylphenyl)-3-oxo-1-piperazinecarboxylate (500 mg, 1.354 mmol) in Dichloromethane (DCM) (12 ml) at RT was added the Trifluoroacetic acid (TFA) (4 ml), and the reaction stirred at RT for 6 hours. The solvent was evaporated in vacuo and the residue was dissolved in MeOH and purified by SCX, eluting with MeOH and then with 2M NH₃/MeOH. The basic fractions were combined and solvent evaporated in vacuo to give a pale yellow oil, 1-(5-bromo-2-methylphenyl)-2-piperazinone (260 mg, 0.966 mmol, 71.3% yield).

### Example 54

### 4-[(2,3-dichlorophenyl)carbonyl]-1-[2-methyl-5-(4-morpholinyl)phenyl]-2-piperazinone (E54)

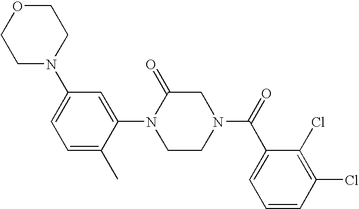

4-[(2,3-Dichlorophenyl)carbonyl]-1-[2-methyl-5-(4-morpholinyl)phenyl]-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 1-[2-methyl-5-(4-morpholinyl)phenyl]-2-piperazinone was used in place of 1-(2-chloro-4-fluorophenyl)-2-piperazinone. [M+H]⁺=449.98, retention time=2.58 minutes.

The 1-[2-methyl-5-(4-morpholinyl)phenyl]-2-piperazinone was in turn prepared using the method described in Example 4 for the preparation of 1-(2-methylphenyl)-2-piperazinone but using 2-methyl-5-(4-morpholinyl)aniline in the place of o-toluidine.

2-Methyl-5-(4-morpholinyl)aniline was prepared in the following manner: Potassium phosphate (8500 mg, 40.0 mmol), L-proline (461 mg, 4.00 mmol) & copper(I) iodide (381 mg, 2.002 mmol) were added to a solution of 5-bromo-2-methylaniline (2.5 ml, 20.02 mmol) and morpholine (2.62 ml, 30.0 mmol) in Dimethyl Sulfoxide (DMSO) (15 ml) under Argon and stirred at 120° C. overnight.

Reaction stirred at 120° C. for a further 6 hours. Reaction allowed to cool and diluted with water (˜40 mL), extracted with EtOAc (×3) organic layer separated and washed with water (×2) which was back extracted with EtOAc (×2). Combined organics dried over Na₂SO₄, filtered and reduced under vacuum to leave a brown oil. Purified by SP4 chromatography to leave a light brown solid. 512 mg. Taken onto next step without further purification.

### Example 55

### 4-{[2-chloro-3-(trifluoromethyl)phenyl]carbonyl}-1-[2-methyl-5-(4-morpholinyl)phenyl]-2-piperazinone (E55)

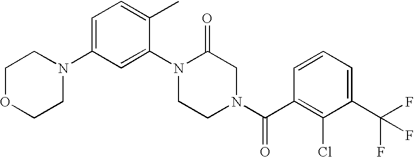

4-{[2-Chloro-3-(trifluoromethyl)phenyl]carbonyl}-1-[2-methyl-5-(4-morpholinyl)phenyl]-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 1-[2-methyl-5-(4-morpholinyl)phenyl]-2-piperazinone and 2-chloro-3-(trifluoromethyl)benzoyl chloride were used in place of 1-(2-chloro-4-fluorophenyl)-2-piperazinone and 2,3-dichlorobenzoyl chloride respectively.

[M+H]⁺ 482, retention time 2.48 minutes.

The 1-[2-methyl-5-(4-morpholinyl)phenyl]-2-piperazinone was in turn prepared using the method described in Example 4 for the preparation of 1-(2-methylphenyl)-2-piperazinone but using 2-methyl-5-(4-morpholinyl)aniline (CAS [1007211-91-7], see WO 2008018426A1 for preparation) in the place of o-toluidine.

### Example 56

### 1-(2-chloro-4-fluorophenyl)-4-{[2-fluoro-3-(trifluoromethyl)phenyl]carbonyl}-2-piperazinone (E56)

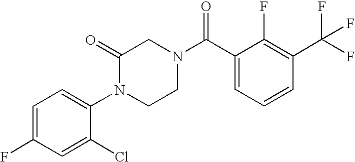

1-(2-Chloro-4-fluorophenyl)-4-{[2-fluoro-3-(trifluoromethyl)phenyl]carbonyl}-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 2-fluoro-3-(trifluoromethyl)benzoyl chloride was used in place of 2,3-dichlorobenzoyl chloride. [M+H]⁺=418.85, retention time=1.03 minutes (2 minute method).

### Example 57

### 4-{[2-chloro-3-(trifluoromethyl)phenyl]carbonyl}-1-{4-fluoro-2-[(1-methylethyl)oxy]phenyl}-2-piperazinone (E57)

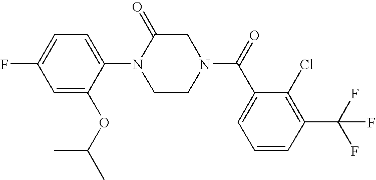

4-{[2-Chloro-3-(trifluoromethyl)phenyl]carbonyl}-1-{4-fluoro-2-[(1-methylethyl)oxy]phenyl}-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 1-{4-fluoro-2-[(1-methylethyl)oxy]phenyl}-2-piperazinone and 2-fluoro-3-(trifluoromethyl)benzoyl chloride were used in place of 1-(2-chloro-4-fluorophenyl)-2-piperazinone and 2,3-dichlorobenzoyl chloride respectively. [M+H]⁺=458.99, retention time=3.02 minutes.

The 4-{[2-chloro-3-(trifluoromethyl)phenyl]carbonyl}-1-{4-fluoro-2-[(1-methylethyl)oxy]phenyl}-2-piperazinone was in turn prepared using the method described in Example 4 for the preparation of 1-(2-methylphenyl)-2-piperazinone but using 4-fluoro-2-[(1-methylethyl)oxy]aniline in the place of o-toluidine.

### Example 58

### 4-[(2,3-dichlorophenyl)carbonyl]-1-{4-fluoro-2-[(1-methylethyl)oxy]phenyl}-2-piperazinone (E58)

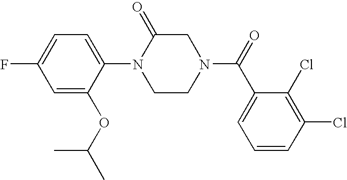

4-[(2,3-Dichlorophenyl)carbonyl]-1-{4-fluoro-2-[(1-methylethyl)oxy]phenyl}-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 1-{4-fluoro-2-[(1-methylethyl)oxy]phenyl}-2-piperazinone was used in place of 1-(2-chloro-4-fluorophenyl)-2-piperazinone.

[M+H]⁺=424.93, retention time=2.94 minutes.

The 4-{[2-chloro-3-(trifluoromethyl)phenyl]carbonyl}-1-{4-fluoro-2-[(1-methylethyl)oxy]phenyl}-2-piperazinone was in turn prepared using the method described in Example 4 for the preparation of 1-(2-methylphenyl)-2-piperazinone but using 4-fluoro-2-[(1-methylethyl)oxy]aniline in the place of o-toluidine.

### Example 59

### 1-(2-chloro-4-fluorophenyl)-4-[(3-chloro-2-fluorophenyl)carbonyl]-2-piperazinone (E59)

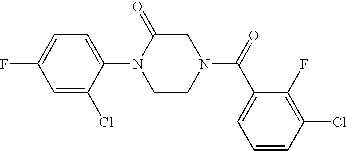

1-(2-Chloro-4-fluorophenyl)-4-[(3-chloro-2-fluorophenyl)carbonyl]-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but 3-chloro-2-fluorobenzoic acid was used in place of the 2-chloro-3-(trifluoromethyl)benzoic acid. [M+H]⁺=384.9, retention time=2.70 minutes.

### Example 60

### 4-[(3-chloro-2,4-difluorophenyl)carbonyl]-1-(2-chloro-4-fluorophenyl)-2-piperazinone (E60)

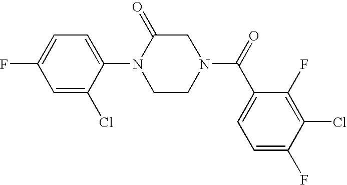

4-[(3-Chloro-2,4-difluorophenyl)carbonyl]-1-(2-chloro-4-fluorophenyl)-2-piperazinone was prepared in a manner analogous to that described above for the synthesis of 1-(2-Chloro-4-fluorophenyl)-4-{[2-chloro-3-(trifluoromethyl)phenyl]carbonyl}-2-piperazinone in Example 10 but 3-chloro-2,4-difluorobenzoic acid was used in place of the 2-chloro-3-(trifluoromethyl)benzoic acid. [M+H]⁺=402.9, retention time=2.77 minutes.

### Example 62

4-{[2-chloro-3-(trifluoromethyl)phenyl]carbonyl}-1-(2,2,2-trifluoroethyl)-2-piperazinone (E62)

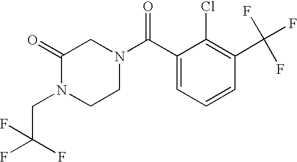

4-{[2-Chloro-3-(trifluoromethyl)phenyl]carbonyl}-1-(2,2,2-trifluoroethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 1,1,1-trifluoro-2-iodoethane in place of iodoethane.

LC/MS=389/391 (M+H)⁺, retention time=2.64.

### Example 63

### 4-{[2-chloro-3-(trifluoromethyl)phenyl]carbonyl}-1-[2-(methyloxy)ethyl]-2-piperazinone (E63)

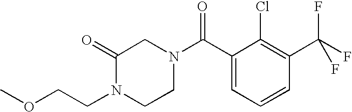

4-{[2-Chloro-3-(trifluoromethyl)phenyl]carbonyl}-1-[2-(methyloxy)ethyl]-2-piperazinone was prepared in a manner analogous to that described above for the synthesis of 4-{[2-chloro-3-(trifluoromethyl)phenyl]carbonyl}-1-ethyl-2-piperazinone in Example 42 but using 2-bromoethyl methyl ether in place of iodoethane.

LC/MS=365/367 (M+H)⁺, retention time=2.26 (5 minute).

### Example 64

### 4-{[2-chloro-3-(trifluoromethyl)phenyl]carbonyl}-1-[5-iodo-2-(methyloxy)phenyl]-2-piperazinone (E64)

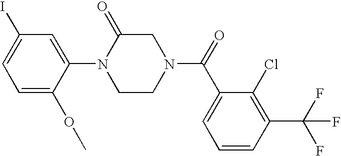

4-{[2-Chloro-3-(trifluoromethyl)phenyl]carbonyl}-1-[5-iodo-2-(methyloxy)phenyl]-2-piperazinone was prepared in a manner analogous to that described above for the synthesis of 1-(2-chloro-4-fluorophenyl)-4-[(2,3-dichlorophenyl)carbonyl]-2-piperazinone in Example 11 but 1-[5-iodo-2-(methyloxy)phenyl]-2-piperazinone and 2-fluoro-3-(trifluoromethyl)benzoyl chloride were used in place of 1-(2-chloro-4-fluorophenyl)-2-piperazinone and 2,3-dichlorobenzoyl chloride respectively. [M+H]⁺=539.0, retention time=3.03 minutes.

The 1-[5-iodo-2-(methyloxy)phenyl]-2-piperazinone was in turn prepared using the method described in Example 4 for the preparation of 1-(2-methylphenyl)-2-piperazinone but using [5-iodo-2-(methyloxy)phenyl]amine in the place of o-toluidine.

### Example 65

### (4-{[2-chloro-3-(trifluoromethyl)phenyl]carbonyl}-2-oxo-1-piperazinyl)acetonitrile (E65)

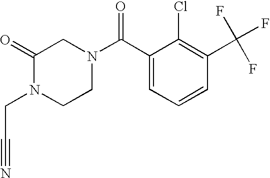

(4-{[2-Chloro-3-(trifluoromethyl)phenyl]carbonyl}-2-oxo-1-piperazinyl)acetonitrile was prepared in a manner analogous to that described above for the synthesis of 4-{[2-chloro-3-(trifluoromethyl)phenyl]carbonyl}-1-ethyl-2-piperazinone in Example 42 but using iodoacetonitrile in place of iodoethane.

LC/MS=346/348 (M+H)⁺, retention time=2.28 min.

### Example 66

### 4-{[2-chloro-3-(trifluoromethyl)phenyl]carbonyl}-1-(cyclohexyl methyl)-2-piperazinone (E66)

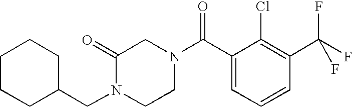

4-{[2-Chloro-3-(trifluoromethyl)phenyl]carbonyl}-1-(cyclohexylmethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using (bromomethyl)cyclohexane in place of iodoethane.

LC/MS=403/405 (M+H)⁺, retention time=3.03 min.

### Example 67

### 4-{[2-chloro-3-(trifluoromethyl)phenyl]carbonyl}-1-(4-pyridinyl methyl)-2-piperazinone (E67)

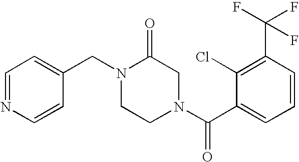

4-{[2-Chloro-3-(trifluoromethyl)phenyl]carbonyl}-1-(4-pyridinylmethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 4-(bromomethyl)pyridine hydrochloride in place of iodoethane.

LC/MS=398/400 (M+H)⁺, retention time=1.66 min.

### Example 68

### 4-{[2-chloro-3-(trifluoromethyl)phenyl]carbonyl}-1-(3-pyridinyl methyl)-2-piperazinone hydrochloride (E68)

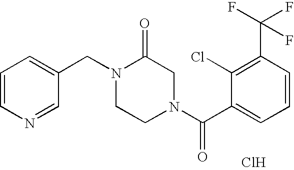

4-{[2-Chloro-3-(trifluoromethyl)phenyl]carbonyl}-1-(3-pyridinylmethyl)-2-piperazinone hydrochloride was prepared in a manner analogous to that described above for the synthesis of 4-{[2-chloro-3-(trifluoromethyl)phenyl]carbonyl}-1-ethyl-2-piperazinone in Example 42 but using 3-(bromomethyl)pyridine hydrobromide in place of iodoethane.

LC/MS=398/400 (M+H)⁺, retention time=1.35 minutes.

### Example 69

### 4-{[2-chloro-3-(trifluoromethyl)phenyl]carbonyl}-1-(2-pyridinyl methyl)-2-piperazinone (E69)

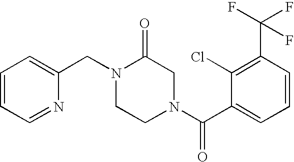

4-{[2-Chloro-3-(trifluoromethyl)phenyl]carbonyl}-1-(2-pyridinylmethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 2-(bromomethyl)pyridine hydrobromide in place of iodoethane.

LC/MS=398/400 (M+H)⁺, retention time=1.72 minutes.

### Example 70

### 4-{[2-chloro-3-(trifluoromethyl)phenyl]carbonyl}-1-(tetrahydro-2-furanyl methyl)-2-piperazinone (E70)

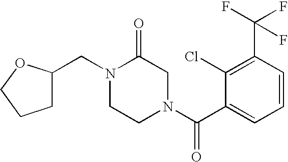

4-{[2-Chloro-3-(trifluoromethyl)phenyl]carbonyl}-1-(tetrahydro-2-furanylmethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using 2-(bromomethyl)tetrahydrofuran in place of iodoethane.

LC/MS=391/393 (M+H)⁺, retention time=2.11 minutes.

### Example 71

### 3-(4-{[2-chloro-3-(trifluoromethyl)phenyl]carbonyl}-2-oxo-1-piperazinyl)propanenitrile (E71)

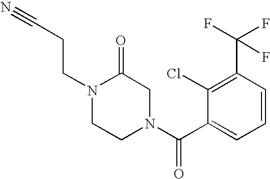

3-(4-{[2-Chloro-3-(trifluoromethyl)phenyl]carbonyl}-2-oxo-1-piperazinyl)propanenitrile was prepared in a manner analogous to that described above for the synthesis of 4-{[2-chloro-3-(trifluoromethyl)phenyl]carbonyl}-1-ethyl-2-piperazinone in Example 42 but using 3-bromopropanenitrile in place of iodoethane.

LC/MS=360/362 (M+H)⁺, retention time=2.01 minutes.

### Example 72

### 2-(4-{[2-chloro-3-(trifluoromethyl)phenyl]carbonyl}-2-oxo-1-piperazinyl)propanenitrile (E72)

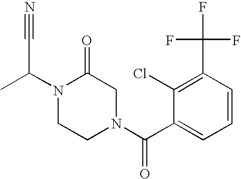

2-(4-{[2-Chloro-3-(trifluoromethyl)phenyl]carbonyl}-2-oxo-1-piperazinyl)propanenitrile was prepared in a manner analogous to that described above for the synthesis of 4-{[2-chloro-3-(trifluoromethyl)phenyl]carbonyl}-1-ethyl-2-piperazinone in Example 42 but using 2-chloropropanenitrile in place of iodoethane.

LC/MS=360/362 (M+H)⁺, retention time=2.23 minutes.

### Example 73

### 4-{[2-chloro-3-(trifluoromethyl)phenyl]carbonyl}-1-[2-methyl-5-(1-pyrrolidinyl)phenyl]-2-piperazinone (E73)

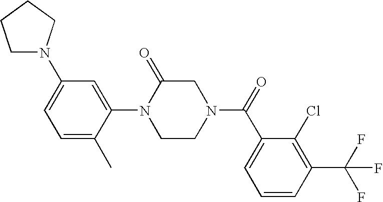

To 1[2-methyl-5-(1-pyrrolidinyl)phenyl]-2-piperazinone (48 mg, 0.185 mmol) in dichloromethane (DCM) (3 ml) was added first the polymer-bound triethylamine (87 mg, 0.278 mmol) and then the 2-chloro-3-(trifluoromethyl)benzoyl chloride (50 mg, 0.206 mmol), and the reaction stirred at RT for 2 hours. NaHCO₃ (sat., aq.) (˜5 ml) was added and then the solution filtered through a phase separator cartridge, washing with DCM. The DCM filtrate was evaporated in vacuo to give a dark orange oil.

The crude product was purified by HPLC. Relevant fractions were combined and solvent evaporated in vacuo to give a yellow gum which was triturated with isohexane and the solid filtered off to give an off-white solid, 4-{[2-chloro-3-(trifluoromethyl)phenyl]carbonyl}-1-[2-methyl-5-(1-pyrrolidinyl)phenyl]-2-piperazinone (23 mg, 0.047 mmol, 25.3% yield).

[M+H]⁺ 466, retention time 2.69 minutes.

The 1-[2-methyl-5-(1-pyrrolidinyl)phenyl]-2-piperazinone used in the above synthesis was prepared as follows:

i) 5-Bromo-2-methylaniline (6 g, 32.2 mmol) in Tetrahydrofuran (THF) (60 ml) was cooled to ˜5° C. in an ice/water bath, and then a solution of potassium carbonate (12.26 g, 89 mmol) in Water (30 ml) was added. The chloroacetyl chloride (3.23 ml, 40.3 mmol) was then added dropwise over 15 minutes to the rapidly stirred bi-phasic solution. The reaction was allowed to warm to RT while stirring for 1 hour, and then the organic layer was separated. The organic layer was cooled to ˜5° C. again and then the ETHANOLAMINE (7 ml, 116 mmol) was added. The reaction was warmed to RT and stirred at RT overnight. The reaction mixture was heated to 50° C. and stirred at 50° C. for 4 hours. After cooling to RT, EtOAc (40 ml) and water (20 ml) were added. Product appeared to crash out in EtOAc layer, so organic layer was separated and then filtered and dried, to give the product as a white solid, N1-(5-bromo-2-methylphenyl)-N2-(2-hydroxyethyl)glycinamide (8.05 g, 28.0 mmol, 87% yield).

M+H 287, 289; retention time 0.91 mins.

ii) To a part-suspension of N1-(5-bromo-2-methylphenyl)-N2-(2-hydroxyethyl)glycinamide (7.05 g, 24.55 mmol) in Dichloromethane (DCM) (150 ml) and Tetrahydrofuran (THF) (150 ml) at 0° C. was added the BOC-Anhydride (6.27 ml, 27.0 mmol) and then the triethylamine (5 ml, 35.9 mmol). The reaction mixture was allowed to warm to RT and stirred at RT overnight. The solvent was removed under vacuum, and then ether was added. The resulting solid was filtered off and the filtrate was evaporated in vacuo to give a colourless oil, 1,1-dimethylethyl {2-[(5-bromo-2-methylphenyl)amino]-2-oxoethyl}(2-hydroxyethyl)carbamate (9.5 g, 24.53 mmol, 100% yield).
iii) Methanesulfonyl chloride (0.211 ml, 2.71 mmol) was added to a stirred solution of 1,1-dimethylethyl {2-[(5-bromo-2-methylphenyl)amino]-2-oxoethyl}(2-hydroxyethyl)carbamate (1 g, 2.58 mmol) in Dichloromethane (DCM) (10 ml) and triethylamine (0.396 ml, 2.84 mmol). The reaction mixture was stirred at RT for 1 hour, and then a further 0.2 ml of triethylamine and 0.1 ml of MsCl were added and the reaction stirred at RT overnight. DCM and NaHCO₃ (sat., aq.) were added, and the product extracted into DCM (×2) and then the combined organic layers were dried over magnesium sulphate. The solvent was evaporated in vacuo to give a pale orange oil. The crude product was purified by column chromatography on silica gel, eluting with 50% EtOAc/iso-hexane. Relevant fractions were combined and solvent evaporated in vacuo to give a colourless oil, 2-({2-[(5-bromo-2-methylphenyl)amino]-2-oxoethyl}{[(1,1-dimethylethyl)oxy]carbonyl}amino)ethyl methanesulfonate (450 mg, 0.967 mmol, 37.4% yield).
iv) To 2-({2-[(5-bromo-2-methylphenyl)amino]-2-oxoethyl}{[(1,1-dimethylethyl)oxy]carbonyl}amino)ethyl methanesulfonate (5.3 g, 11.39 mmol) in N,N-Dimethylformamide (DMF) (45 ml) was added portionwise the sodium hydride (60% dispersion) (500 mg, 12.50 mmol). The reaction mixture was stirred at RT under argon overnight. MeOH (3 ml) was added and then reaction mixture stirred at RT for 10 minutes and then solvent evaporated in vacuo. The residue was dissolved in DCM (20 ml) and NaHCO₃ (sat., aq.) (20 ml) and the product extracted into DCM (×2). The combined organic extracts were dried over magnesium sulphate and then solvent evaporated in vacuo and the residue triturated with Et₂O to give a white solid, 1,1-dimethylethyl 4-(5-bromo-2-methylphenyl)-3-oxo-1-piperazinecarboxylate (1.42 g, 3.85 mmol, 33.8% yield).
v) A mixture of 1,1-dimethylethyl 4-(5-bromo-2-methylphenyl)-3-oxo-1-piperazinecarboxylate (200 mg, 0.542 mmol) and pyrrolidine (0.090 ml, 1.083 mmol) in Toluene (4 ml) was treated with sodium tert-butoxide (78 mg, 0.812 mmol), BINAP (54.0 mg, 0.087 mmol) and Pd₂(dba)₃ (40 mg, 0.044 mmol), and the reaction heated at reflux (˜115° C.) under argon for 2 hours. The reaction mixture was allowed to cool to RT and then was diluted with EtOAc (20 ml) and water (20 ml). The product was extracted into EtOAc (×2), and then the combined organic extracts were washed with water (×1) (20 ml), brine (×1) (20 ml) and then dried over magnesium sulphate. The solvent was evaporated in vacuo to give a dark yellow oil.

The crude product was purified by flash-silica gel chromatography, eluting with 0-100% EtOAc/iso-hexane. Relevant fractions were combined and solvent evaporated in vacuo to give a yellow oil, 1,1-dimethylethyl 4-[2-methyl-5-(1-pyrrolidinyl)phenyl]-3-oxo-1-piperazinecarboxylate (70 mg, 0.195 mmol, 36.0% yield).

vi) To 1,1-dimethylethyl 4-[2-methyl-5-(1-pyrrolidinyl)phenyl]-3-oxo-1-piperazinecarboxylate (70 mg, 0.195 mmol) in Dichloromethane (DCM) (2 ml) at RT was added the Trifluoroacetic acid (TFA) (1 ml), and the reaction mixture stirred at RT for 1 hour. The solvent was then removed in vacuo and the residue was dissolved in MeOH. The reaction was purified by SCX, eluting first with MeOH and then with 2M NH₃/MeOH. The basic fractions were combined and solvent evaporated in vacuo to give a dark yellow oil, 1-[2-methyl-5-(1-pyrrolidinyl)phenyl]-2-piperazinone (50 mg, 0.193 mmol, 99% yield).

### Example 74

### 4-{[2-chloro-3-(trifluoromethyl)phenyl]carbonyl}-1-[2-methyl-5-(6-methyl-3-pyridinyl)phenyl]-2-piperazinone hydrochloride (E74)

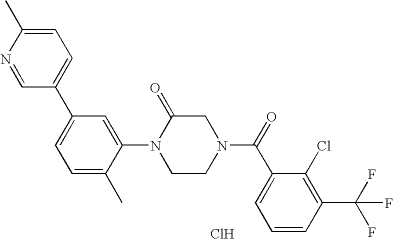

A mixture of 1-(5-bromo-2-methylphenyl)-4-{[2-chloro-3-(trifluoromethyl)phenyl]carbonyl}-2-piperazinone (100 mg, 0.210 mmol, prepared as described above for Example 53), (6-methyl-3-pyridinyl)boronic acid (57.6 mg, 0.420 mmol) and sodium carbonate (111 mg, 1.051 mmol) in 1,2-Dimethoxyethane (DME) (2 ml) and Water (2.000 ml) was treated with Pd(Ph₃P)₄ (146 mg, 0.126 mmol) and the reaction mixture heated in the microwave at 100° C. (high absorbtion) for 2 hours. The reaction mixture was diluted with EtOAc (15 ml) and NaHCO₃ (sat., aq.) (15 ml) and the product was extracted into EtOAc (×2). The combined organic layers were washed with water (15 ml), brine (15 ml) and then dried over magnesium sulphate. The solvent was evaporated in vacuo to give a dark brown oil.

The crude product was purified by HPLC. Relevant fractions were combined and solvent evaporated in vacuo to give a colourless residue (the formate salt), which was dissolved in DCM (10 ml) and NaHCO₃ (sat., aq.). Product was extracted into the DCM and then the DCM was dried over magnesium sulphate and solvent evaporated in vacuo to give a white solid/gum. This was dissolved in 1 ml DCM and treated with 0.5 ml 1M HCl in Et₂O, and the reaction mixture stirred at RT for 30 minutes. The solvent was removed in vacuo and then co-evaporated with Et₂O to give an off-white solid, 4-{[2-chloro-3-(trifluoromethyl)phenyl]carbonyl}-1-[2-methyl-5-(6-methyl-3-pyridinyl)phenyl]-2-piperazinone. HCl (35 mg, 0.060 mmol, 28.6% yield), [M+H]⁺ 488, retention time 1.76 minutes.

### Example 75

4-{[2-chloro-3-(trifluoromethyl)phenyl]carbonyl}-1-[2-methyl-3-(1-pyrrolidinyl)phenyl]-2-piperazinone (E75)

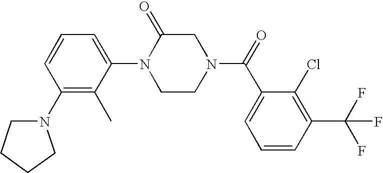

4-{[2-Chloro-3-(trifluoromethyl)phenyl]carbonyl}-1-[2-methyl-3-(1-pyrrolidinyl)phenyl]-2-piperazinone was prepared in a manner analogous to that described above for the synthesis of 4-{[2-chloro-3-(trifluoromethyl)phenyl]carbonyl}-1-[2-methyl-5-(1-pyrrolidinyl)phenyl]-2-piperazinone in Example 73 but using 3-Bromo-2-methylaniline in place of 5-Bromo-2-methylaniline.

[M+H]⁺ 466; retention time 2.02 minutes.

### Example 76

### 1-[2-chloro-5-(4-morpholinyl)phenyl]-4-[(2,3-dichlorophenyl)carbonyl]-2-piperazinone (E76)

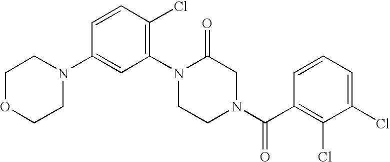

To 1-[2-chloro-5-(4-morpholinyl)phenyl]-2-piperazinone (250 mg, 0.845 mmol) in dichloromethane (10 ml) was added first the polymer-bound triethylamine (396 mg, 1.268 mmol) and then the 2,3-dichlorobenzoyl chloride (0.131 ml, 0.972 mmol), and the reaction stirred at room temperature overnight. Saturated sodium hydrogen carbonate solution was added, stirred for 10 minutes and the organic layer was separated through a hydrophobic frit and reduced under vacuum. The residue was purified by SP4 silica gel chromatography with a 10-100% ethyl acetate/isohexane gradient, eluting in 100% ethylacetate, to leave a white solid.

1-[2-chloro-5-(4-morpholinyl)phenyl]-4-[(2,3-dichlorophenyl)carbonyl]-2-piperazinone (277 mg, 0.579 mmol, 68.5% yield)

LC/MS [M+H]⁺=468.02, retention time=2.53 minutes

The 1-[2-chloro-5-(4-morpholinyl)phenyl]-2-piperazinone used in the above synthesis was prepared as follows:

i) N-Chlorosuccinimide (6.51 g, 48.8 mmol) in chloroform (200 ml) was added slowly to a solution of 3-(4-morpholinyl)aniline (8.69 g, 48.8 mmol) in chloroform (200 ml) at room temperature and stirred for 3 hours. 880 ammonia solution was added and the reaction stirred for 30 minutes after which the organic layer was separated by hydrophobic frit and reduced under vacuum. The crude material was purified by SP4 silica gel chromatography eluting with 0-25% ethylacetate in isohexane (Crude material split onto 4 columns. 250 mL @ 0%, 250 mL @ 5%, 250 mL @ 10%, 750 mL @ 15%, 750 mL @ 20% & 250 mL @ 25%). Pure fractions were combined and reduced under vacuum to leave a yellow solid. 2-chloro-5-(4-morpholinyl)aniline (2.6 g, 11.98 mmol, 24.57% yield)

LC/MS [M+H]⁺ 212.92, retention time=0.82

ii) 2-Chloro-5-(4-morpholinyl)aniline (3.25 g, 15.28 mmol) in tetrahydrofuran (30 ml) was cooled to ˜5° C. in an ice/water bath, and then a solution of potassium carbonate (5.81 g, 42.0 mmol) in water (15 ml) was added. The chloroacetyl chloride (1.530 ml, 19.10 mmol) was then added dropwise over 10 minutes to the rapidly stirred bi-phasic solution. The reaction was allowed to warm to room temperature while stirring for 1 hour. The organic layer was separated and cooled to ˜5° C., ethanolamine (0.924 ml, 15.28 mmol) was then added and the reaction was allowed to warm up to room temperature and stirred for 2 hours then stirred at 50° C. overnight. Ethanolamine (0.924 ml, 15.28 mmol) was added and the reaction was stirred at reflux for a further 3 hours. Sodium iodide (2.291 g, 15.28 mmol) was then added and the reaction stirred at reflux for 2 hours. The reaction was allowed to cool and partitioned between ethylacetate and water. The aqueous layer was extracted with ethylacetate and the combined organic layers were washed with water and brine and dried over sodium sulphate. The crude material was purified by SP4 silica gel chromatography eluting with 0-10% methanol in dichloromethane to leave a yellow oil which solidified under high vacuum to leave a yellow solid. N1-[2-chloro-5-(4-morpholinyl)phenyl]-N2-(2-hydroxyethyl)glycinamide (2.85 g, 8.63 mmol, 56.5% yield)

LC/MS [M+H]⁺=313.97, retention time=0.47.

iii) BOC-Anhydride (2.320 ml, 9.99 mmol) was added to a solution of N˜1˜-[2-chloro-5-(4-morpholinyl)phenyl]-N-2˜-(2-hydroxyethyl)glycinamide (2.85 g, 9.08 mmol) in dichloromethane (50 ml) and stirred at room temperature overnight. The solvent was removed under vacuum and dried under high vacuum to leave a off white foam.

LC/MS [M+H]⁺=414.00, retention time=0.93 minutes.

iv) Methanesulfonyl chloride (2.169 ml, 27.8 mmol) was added to a solution of 1,1-dimethylethyl-2-{[2-chloro-5-(4-morpholinyl)phenyl]amino}-2-oxoethyl)(2-hydroxyethyl)carbamate (3.6 g, 8.70 mmol) in dichloromethane (40 ml) and triethylamine (9.7 ml, 69.6 mmol) and the reaction stirred at room temperature for 1.5 hour. The reaction was partitioned between dichloromethane and saturated sodium hydrogen carbonate solution, the aqueous layer extracted with dichloromethane, separated by hydrophobic frit and the combined organics reduced under vacuum to yield a pink gum; assume 100% conversion. Taken onto next step without purification. LC/MS (JHG11310) shows 84% desired compound [M+H]⁺=491.91, retention time=1.05 minutes.
v) Sodium hydride (60% dispersion in oil) (0.561 g, 14.02 mmol) was added slowly to a solution of 2-((2-{[2-chloro-5-(4-morpholinyl)phenyl]amino}-2-oxoethyl){[(1,1-dimethylethyl)oxy]carbonyl}amino)ethyl methanesulfonate (4.6 g, 9.35 mmol) in N,N-dimethylformamide (20 ml) and stirred at room temperature for overnight. The reaction was quenched with saturated sodium hydrogen carbonate solution and extracted with ethyl acetate (×2). The combined organic layers were reduced under vacuum and purified by SP4 silica gel chromatography eluting with 12-100% ethylacetate in iso-hexane over 40 minutes to leave white solid. 1,1-dimethylethyl 4-[2-chloro-5-(4-morpholinyl)phenyl]-3-oxo-1-piperazinecarboxylate (2.2 g, 5.45 mmol, 58.2% yield)

LC/MS [M+H]⁺=395.83, retention time=1.08 minutes.

vi) A solution of 1,1-dimethylethyl 4-[2-chloro-5-(4-morpholinyl)phenyl]-3-oxo-1-piperazinecarboxylate (2.2 g, 5.56 mmol) in dichloromethane (15 ml) was treated with Trifluoroacetic acid (4 ml, 51.9 mmol) and stirred at room temperature for 6 hours. The solvent was removed under vacuum azeotroping with toluene and dried overnight under high vacuum. The dark pink gum was dissolved in methanol and loaded onto an SCX cartridge, washed through with methanol, and eluted with 2N NH₃ methanol solution. The product containing fraction was reduced under vacuum to leave a pale yellow solid. 1-[2-chloro-5-(4-morpholinyl)phenyl]-2-piperazinone (1.5 g, 4.97 mmol, 89% yield).

LC/MS [M+H]=295.96, retention time=0.46 (2 minute run)

### Example 77

### 1-[2-chloro-5-(4-morpholinyl)phenyl]-4-[(2,4,6-trichlorophenyl)carbonyl]-2-piperazinone (E77)

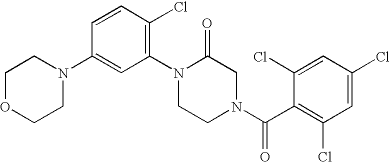

1-[2-chloro-5-(4-morpholinyl)phenyl]-4-[(2,4,6-trichlorophenyl)carbonyl]-2-piperazinone was prepared in a manner analogous to that described above for the synthesis of 1-[2-chloro-5-(4-morpholinyl)phenyl]-4-[(2,3-dichlorophenyl)carbonyl]-2-piperazinone in Example 76 but using 2,4,6-trichlorobenzoyl chloride in place of 2,3-dichlorobenzoyl chloride.

LC/MS [M+H]⁺=501.94, retention time 2.69-2.74 minutes; 2 peaks-different rotamers.

### Example 78

### 1-[2-chloro-5-(4-morpholinyl)phenyl]-4-{[2-chloro-3-(trifluoromethyl)phenyl]-carbonyl}-2-piperazinone (E78)

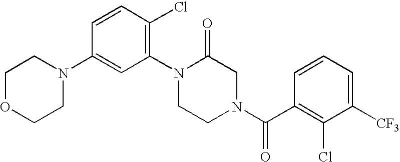

1-[2-chloro-5-(4-morpholinyl)phenyl]-4-{[2-chloro-3-(trifluoromethyl)phenyl]-carbonyl}-2-piperazinone was prepared in a manner analogous to that described above for the synthesis of 1-[2-chloro-5-(4-morpholinyl)phenyl]-4-[(2,3-dichlorophenyl)carbonyl]-2-piperazinone in Example 76 but using 2-chloro-3-(trifluoromethyl)benzoyl chloride in place of 2,3-dichlorobenzoyl chloride.

LC/MS [M+H]⁺=501.94, retention time 2.69-2.74 minutes; 2 peaks-different rotamers

### Example 79

### 1-[2-chloro-3-(4-morpholinyl)phenyl]-4-{[2-chloro-3-(trifluoromethyl)phenyl]carbonyl}-2-piperazinone (E79)

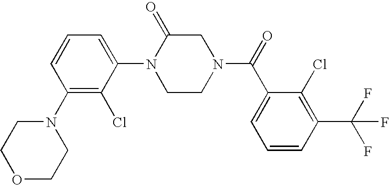

1-[2-chloro-3-(4-morpholinyl)phenyl]-4-{[2-chloro-3-(trifluoromethyl)phenyl]carbonyl}-2-piperazinone was prepared in a manner analogous to that described above for the synthesis of 1-[2-chloro-5-(4-morpholinyl)phenyl]-4-[(2,3-dichlorophenyl)carbonyl]-2-piperazinone in Example 76 but using 2-chloro-3-(trifluoromethyl)benzoyl chloride and 2-chloro-3-(4-morpholinyl)aniline in place of 2,3-dichlorobenzoyl chloride and 2-chloro-5-(4-morpholinyl)aniline respectively.

[M+H]⁺ 502, retention time 2.58 minutes

The 2-chloro-3-(4-morpholinyl)aniline used in the above synthesis was prepared in the following manner:

i) Palladium(ii) acetate (23.74 mg, 0.106 mmol), BINAP (99 mg, 0.159 mmol) and caesium carbonate (517 mg, 1.586 mmol) were combined in tetrahydrofuran (THF) (7 ml) and stirred under argon at room temperature for 30 minutes. 1-bromo-2-chloro-3-nitrobenzene (250 mg, 1.057 mmol) and morpholine (0.276 ml, 3.17 mmol) were added and the reaction heated to reflux at 85° C. under argon for 16 hours. LCMS (N4669-19-A1:HHJ22983) shows desired product at 1.00 min (32%).

The reaction mixture was diluted with ethyl acetate (20 ml) and the catalyst residues filtered off. The filtrate was concentrated in vacuo to yield an orange oil. The crude material was dissolved in a minimum of DCM and loaded onto a 25+S Biotage cartridge. This was eluted with a 0-100% gradient of ethyl acetate in hexane using the SP4. The product did not elute cleanly, but product fractions were concentrated in vacuo to yield crude 4-(2-chloro-3-nitrophenyl)morpholine (0.1498 g, 0.617 mmol, 58.4% yield) as a yellow solid. The crude material will be taken forward to the next reaction.

ii) 4-(2-chloro-3-nitrophenyl)morpholine (430 mg, 1.772 mmol) was dissolved in Acetic Acid (25 ml) and iron (granules) (990 mg, 17.72 mmol) was added. The mixture was warmed to 50° C. for 18 hours. After cooling to RT, the reaction mixture was filtered, washing with EtOAc, and then the filtrate concentrated in vacuo. The residue was partitioned between ethyl acetate (60 ml) and water (50 ml). The product was extracted into EtOAc (×2) and then the combined organic extracts dried over magnesium sulphate. The solvent was evaporated in vacuo to give a dark brown solid, 2-chloro-3-(4-morpholinyl)aniline (340 mg, 1.599 mmol, 90% yield).

### Example 80

### 4-[(2,3-dichlorophenyl)carbonyl]-1-[2-methyl-3-(4-morpholinyl)phenyl]-2-piperazinone (E80)

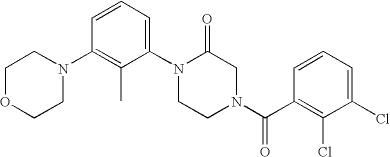

4-[(2,3-Dichlorophenyl)carbonyl]-1-[2-methyl-3-(4-morpholinyl)phenyl]-2-piperazinone was prepared in a manner analogous to that described above for the synthesis of 1-[2-chloro-3-(4-morpholinyl)phenyl]-4-{[2-chloro-3-(trifluoromethyl)phenyl]carbonyl}-2-piperazinone in Example 79 but using 2,3-dichlorobenzoyl chloride and 6-bromo-2-nitrotoluene in place of 2-chloro-3-(trifluoromethyl)benzoyl chloride and 1-bromo-2-chloro-3-nitrobenzene respectively.

LC/MS [M+H]⁺=448.09, retention time=2.46 minutes.

### Example 81

### 4-{[2-chloro-3-(trifluoromethyl)phenyl]carbonyl}-1-[2-methyl-3-(4-morpholinyl)phenyl]-2-piperazinone (E81)

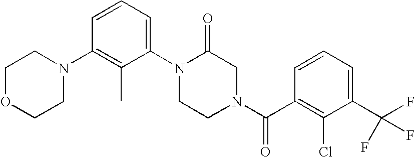

4-{[2-Chloro-3-(trifluoromethyl)phenyl]carbonyl}-1-[2-methyl-3-(4-morpholinyl)phenyl]-2-piperazinone was prepared in a manner analogous to that described above for the synthesis of 1-[2-chloro-3-(4-morpholinyl)phenyl]-4-{[2-chloro-3-(trifluoromethyl)phenyl]carbonyl}-2-piperazinone in Example 79 but using 6-bromo-2-nitrotoluene in place of 1-bromo-2-chloro-3-nitrobenzene.

LC/MS [M+H]⁺=482.09, retention time=2.59 minutes (5 minute run)

### Example 82

### 1-[2-methyl-3-(4-morpholinyl)phenyl]-4-[(2,4,6-trichlorophenyl)carbonyl]-2-piperazinone (E82)

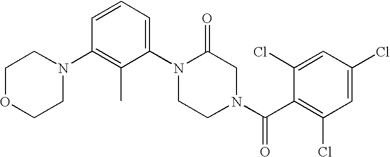

4-[(2,3-Dichlorophenyl)carbonyl]-1-[2-methyl-3-(4-morpholinyl)phenyl]-2-piperazinone was prepared in a manner analogous to that described above for the synthesis of 1-[2-chloro-3-(4-morpholinyl)phenyl]-4-{[2-chloro-3-(trifluoromethyl)phenyl]carbonyl}-2-piperazinone in Example 79 but using 2,4,6-trichlorobenzoyl chloride and 6-bromo-2-nitrotoluene in place of 2-chloro-3-(trifluoromethyl)benzoyl chloride and 1-bromo-2-chloro-3-nitrobenzene respectively.

LC/MS [M+H]⁺=482.03, retention time 2.64-2.68 minutes; 2 peaks different rotamers.

### Example 83

### 4-{[2-chloro-3-(trifluoromethyl)phenyl]carbonyl}-1-[5-(3-hydroxy-1-pyrrolidinyl)-2-methyl phenyl]-2-piperazinone (E83)

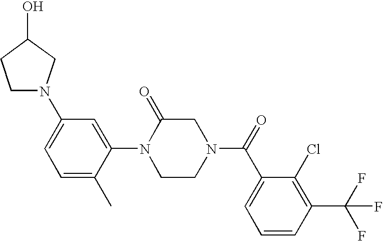

To 1-[5-(3-hydroxy-1-pyrrolidinyl)-2-methylphenyl]-2-piperazinone (30 mg, 0.109 mmol) in dichloromethane (DCM) (2 ml) was added polymer-bound triethylamine (109 mg, 0.349 mmol) and then 2-chloro-3-(trifluoromethyl)benzoyl chloride (30 mg, 0.123 mmol). The reaction mixture was stirred at RT for 2 hours and then filtered through a hydrophobic frit, washing with DCM. The DCM filtrate was evaporated in vacuo and then the residue was purified by flash-silica gel chromatography, eluting with 0-100% EtOAc/iso-hexane. Relevant fractions were combined and solvent evaporated in vacuo, co-evaporated with Et₂O and iso-hexane to give an off-white solid, 4-{[2-chloro-3-(trifluoromethyl)phenyl]carbonyl}-1-[5-(3-hydroxy-1-pyrrolidinyl)-2-methylphenyl]-2-piperazinone (22 mg, 0.041 mmol, 37.7% yield).

[M+H]⁺ 482, retention time 2.39 minutes

The 1-[5-(3-hydroxy-1-pyrrolidinyl)-2-methylphenyl]-2-piperazinone used in the above synthesis was prepared in the following manner:

i) 5-Bromo-2-methylaniline (6 g, 32.2 mmol) in tetrahydrofuran (THF) (60 ml) was cooled to ˜5° C. in an ice/water bath, and then a solution of potassium carbonate (12.26 g, 89 mmol) in water (30 ml) was added. The chloroacetyl chloride (3.23 ml, 40.3 mmol) was then added dropwise over 15 minutes to the rapidly stirred bi-phasic solution. The reaction was allowed to warm to RT while stirring for 1 hour, and then the organic layer was separated. The organic layer was cooled to ˜5° C. again and then the ethanolamine (7 ml, 116 mmol) was added. The reaction was allowed to warm to RT and stirred at RT overnight. The reaction mixture was then heated to 50° C. and stirred at 50° C. for 3 hours. After cooling to RT, EtOAc (40 ml) and water (20 ml) were added. Product was extracted into EtOAc (×2) and then the combined organic extracts were evaporated in vacuo to give a purple solid, which was triturated with Et₂O to give the product as a white solid, N1-(5-bromo-2-methylphenyl)-N2-(2-hydroxyethyl)glycinamide (7.5 g, 26.1 mmol, 81% yield). M+H 287, 289; retention time 0.97 mins.
ii) To a suspension of N1-(5-bromo-2-methylphenyl)-N2-(2-hydroxyethyl)glycinamide (7.5 g, 26.1 mmol) in dichloromethane (DCM) (350 ml) was added the BOC-Anhydride (6.67 ml, 28.7 mmol), and the reaction mixture was stirred at RT overnight (NB: SM gradually goes into solution over time). The solvent was removed under vacuum, and then Et₂O was added. The resulting solid was filtered off and the filtrate was evaporated in vacuo to give a colourless oil, 1,1-dimethylethyl {2-[(5-bromo-2-methylphenyl)amino]-2-oxoethyl}(2-hydroxyethyl)carbamate (11.3 g, 29.2 mmol, 112% yield).
iii) Methanesulfonyl chloride (3 ml, 38.5 mmol) was added to a stirred solution of 1,1-dimethylethyl {2-[(5-bromo-2-methylphenyl)amino]-2-oxoethyl}(2-hydroxyethyl)carbamate (10.1 g, 26.1 mmol) in Dichloromethane (DCM) (100 ml) and triethylamine (7 ml, 50.2 mmol). The reaction mixture was stirred at RT overnight. DCM and NaHCO₃ (sat., aq.) were added, and the product extracted into DCM (×2) and then the combined organic layers were dried over magnesium sulphate. The solvent was evaporated in vacuo to give a pale orange oil.

The crude product was purified by column chromatography on silica gel, eluting with 50% EtOAc/iso-Hexane. Relevant fractions were combined and solvent evaporated in vacuo to give a colourless oil, 2-({2-[(5-bromo-2-methylphenyl)amino]-2-oxoethyl}{[(1,1-dimethylethyl)oxy]carbonyl}amino)ethyl methanesulfonate (3.4 g, 7.31 mmol, 28.0% yield).

iv) To 2-({2-[(5-bromo-2-methylphenyl)amino]-2-oxoethyl}{[(1,1-dimethylethyl)oxy]carbonyl}amino)ethyl methanesulfonate (3.4 g, 7.31 mmol) in N,N-dimethylformamide (DMF) (30 ml) was added portionwise the sodium hydride (60% dispersion) (350 mg, 8.75 mmol). The reaction mixture was stirred at RT under argon overnight. A further 0.5 equivalents of sodium hydride was added and the reaction mixture left to stir at RT under argon for a further 3 hours. MeOH (˜10 ml) was added and then the reaction mixture stirred at RT for 10 minutes and then the solvent evaporated in vacuo. The residue was dissolved in DCM (30 ml) and NaHCO₃ (sat., aq.) (20 ml) and the product extracted into DCM (×2). The combined organic extracts were dried over magnesium sulphate and then solvent evaporated in vacuo. The residue was purified by flash-silica gel chromatography, eluting with 0-100% EtOAc/iso-Hexane. The relevant fractions were combined and solvent evaporated in vacuo. The residue was triturated with Et₂₀ to give a white solid, 1,1-dimethylethyl 4-(5-bromo-2-methylphenyl)-3-oxo-1-piperazinecarboxylate (950 mg, 2.57 mmol, 35.2% yield).
v) A mixture of 1,1-dimethylethyl 4-(5-bromo-2-methylphenyl)-3-oxo-1-piperazinecarboxylate (200 mg, 0.542 mmol) and 3-pyrrolidinol (0.088 ml, 1.083 mmol) in toluene (4 ml) was treated with sodium tert-butoxide (78 mg, 0.812 mmol), BINAP (54.0 mg, 0.087 mmol) and Pd₂(dba)₃ (40 mg, 0.044 mmol), and the reaction heated at reflux (˜115° C.) under argon overnight. The reaction mixture was allowed to cool to RT and then was diluted with EtOAc (20 ml) and water (20 ml). The product was extracted into EtOAc (×2), and then the combined organic extracts were washed with water (×1) (20 ml), brine (×1) (20 ml) and then dried over magnesium sulphate. The solvent was evaporated in vacuo to give a dark yellow oil.

The crude product was purified by flash-silica gel chromatography, eluting with 0-100% EtOAc/iso-hexane. Product did not elute cleanly, but relevant fractions were combined and solvent evaporated in vacuo to give an orange oil, 1,1-dimethylethyl 4-[5-(3-hydroxy-1-pyrrolidinyl)-2-methylphenyl]-3-oxo-1-piperazinecarboxylate (50 mg, 0.133 mmol, 24.59% yield).

vi) To 1,1-dimethylethyl 4-[5-(3-hydroxy-1-pyrrolidinyl)-2-methylphenyl]-3-oxo-1-piperazinecarboxylate (50 mg, 0.133 mmol) in Dichloromethane (DCM) (2 ml) at RT was added the trifluoroacetic acid (1 ml, 12.98 mmol), and the reaction mixture stirred at RT for 1 hour. The solvent was then removed in vacuo and the residue was dissolved in MeOH. The reaction was purified by SCX, eluting first with MeOH and then with 2M NH₃/MeOH. The basic fractions were combined and solvent evaporated in vacuo to give an orange oil, 1-[5-(3-hydroxy-1-pyrrolidinyl)-2-methylphenyl]-2-piperazinone (30 mg, 0.109 mmol, 82% yield). Crude product taken to the next step.

### Example 84

### 4-{[2-chloro-3-(trifluoromethyl)phenyl]carbonyl}-1-[2-methyl-3-(6-methyl-3-pyridinyl)phenyl]-2-piperazinone (E84)

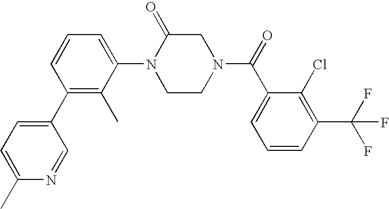

A mixture of 1-(3-bromo-2-methylphenyl)-4-{[2-chloro-3-(trifluoromethyl)phenyl]carbonyl}-2-piperazinone (250 mg, 0.526 mmol, prepared as described in Example 46), (6-methyl-3-pyridinyl)boronic acid (144 mg, 1.051 mmol) and sodium carbonate (279 mg, 2.63 mmol) in 1,2-Dimethoxyethane (DME) (2 ml) and water (2.000 ml) was treated with Pd(Ph₃P)₄ (364 mg, 0.315 mmol) and the reaction mixture heated in the microwave at 100° C. (high absorbtion) for 2 hours. The reaction mixture was diluted with EtOAc (15 ml) and NaHCO₃ (sat., aq.) (15 ml) and the product was extracted into EtOAc (×2). The combined organic layers were washed with water (15 ml), brine (15 ml) and then dried over magnesium sulphate. The solvent was evaporated in vacuo to give a dark brown oil. The crude product was purified by column flash-silica gel chromatography eluting with 0 to 100% EtOAc in iso-hexane. No product was found in the fractions collected, so the product was purified again by flash-silica gel chromatography eluting with 0 to 50% methanol in EtOAc. Relevant fractions were combined and solvent evaporated in vacuo to give a brown solution. The mixture was stirred with charcoal and then filter through celite to give a yellow pale product.

The product was transformed into an hydrochloric acid salt by adding 2 ml of DCM and 1 ml of hydrochloric acid in ether and the solution was left to stir during 1 h at RT. The solvent was evaporated in vacuo, to give a yellow powder. The compound was dried, triturated with ether and then dried again in the oven. The product was dissolved in DMSO and purified by mass-directed automated HPLC.

Product-containing fractions were concentrated under vacuum. The collected fractions were purified by SCX eluting with methanol and then with 2N NH₃/methanol. Ammonia fractions were combined. The solvent was evaporated in vacuo and the product was transformed into an hydrochloric acid salt by adding 2 ml of DCM and 1 ml of hydrochloric acid in ether and the solution was left to stir during 1 h at RT.

The solvent was evaporated in vacuo, to give a pale yellow powder, 4-{[2-chloro-3-(trifluoromethyl)phenyl]carbonyl}-1-[2-methyl-3-(6-methyl-3-pyridinyl)phenyl]-2-piperazinone (65 mg, 0.133 mmol, 25.3% yield). [M+H]⁺=488.08, retention time=1.74 minutes

### Example 85

### 4-{[2-chloro-3-(trifluoromethyl)phenyl]carbonyl}-1-(1-phenylethyl)-2-piperazinone (E85)

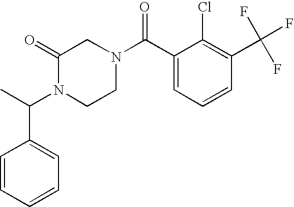

4-{[2-Chloro-3-(trifluoromethyl)phenyl]carbonyl}-1-(1-phenylethyl)-2-piperazinone was prepared in a manner analogous to that described above for the synthesis of 4-{[2-chloro-3-(trifluoromethyl)phenyl]carbonyl}-1-ethyl-2-piperazinone in Example 42 but using (1-bromoethyl)benzene in place of iodoethane.

[M+H]⁺ 411; retention time 2.71 minutes

### Example 86

### 1-(2-chloro-4-fluorophenyl)-4-[(4-chloro-2-fluorophenyl)carbonyl]-2-piperazinone

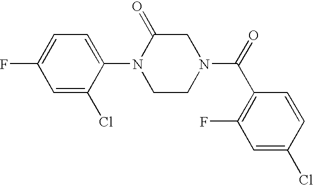

### Example 87

### 1-(2-chloro-4-fluorophenyl)-4-[(2,3-difluorophenyl)carbonyl]-2-piperazinone

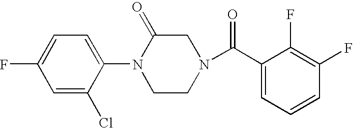

### Mass-Directed Automated HPLC

Where indicated in the above examples, purification by mass-directed automated HPLC was carried out using the following apparatus and conditions:

### Hardware

### Waters 2525 Binary Gradient Module

### Waters 515 Makeup Pump

### Waters Pump Control Module

### Waters 2767 Inject Collect

### Waters Column Fluidics Manager

### Waters 2996 Photodiode Array Detector

### Waters ZQ Mass Spectrometer

Gilson 202 fraction collector
Gilson Aspec waste collector

### Software

Waters MassLynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol:Water 80:20
Needle rinse solvent=Methanol

### Methods

There are five methods used depending on the analytical retention time of the compound of interest. They have a 13.5-minute runtime, which comprises a 10-minute gradient followed by a 3.5 minute column flush and re-equilibration step.

### Large/Small Scale 1.0-1.5=5-30% B

### Large/Small Scale 1.5-2.2=15-55% B

### Large/Small Scale 2.2-2.9=30-85% B

### Large/Small Scale 2.9-3.6=50-99% B

Large/Small Scale 3.6-5.0=80-99% B (in 6 minutes followed by 7.5 minutes flush and re-equilibration)

### Flow Rate

All of the above methods have a flow rate of either 20 mls/min (Small Scale) or 40 mls/min (Large Scale).

### Liquid Chromatography/Mass Spectrometry

Analysis of the above Examples by Liquid Chromatography/Mass Spectrometry (LC/MS) was carried out using the following apparatus and conditions:

### Hardware

### Agilent 1100 Gradient Pump

### Agilent 1100 Autosampler

### Agilent 1100 DAD Detector

### Agilent 1100 Degasser

### Agilent 1100 Oven

### Agilent 1100 Controller

### Waters ZQ Mass Spectrometer

### Sedere Sedex 85

### Software

Waters MassLynx version 4.0 SP2

### Column

The column used is a Waters Atlantis, the dimensions of which are 4.6 mm×50 mm.

The stationary phase particle size is 3 μm.

### Solvents

A: Aqueous solvent=Water+0.05% Formic Acid
B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3 ml/mins.

The injection volume for the generic method is 5 ul.

The column temperature is 30 deg.

The UV detection range is from 220 to 330 nm.

### Pharmacological Data

Compounds or salts of the invention may be tested for in vitro biological activity at the P2X7 receptor in accordance with the following studies:

### Ethidium Accumulation Assay

Studies were performed using NaCl assay buffer of the following composition: 140 mM NaCl, 10 mM HEPES [4-(2-hydroxyethyl)-1-piperazine-1-ethanesulfonic acid], 5 mM N-methyl-D-glucamine, 5.6 mM KCl, 10 mM D-glucose, 0.5 mM CaCl₂ (pH 7.4).

Human Embryonic Kidney (HEK) 293 cells, stably expressing human recombinant P2X7 receptors, were grown in poly-D-lysine pre-treated 96 well plates for 18-24 hours. (The cloning of the human P2X7 receptor is described in U.S. Pat. No. 6,133,434, e.g. see Example 3 therein). The cells were washed twice with 350 μl of the assay buffer, before addition of 50 μl of the assay buffer containing the putative P2X7 receptor antagonist compound. (A small amount of dimethyl sulfoxide, for initially dissolving the compound, is optionally used and present in this 50 μl test compound sample.) The cells were then incubated at room temperature (19-21° C.) for 30 min before addition of ATP and ethidium (100 μM final assay concentration). The ATP concentration was chosen to be close to the EC₈₀ for the receptor type and was 1 mM for studies on the human P2X7 receptor. Incubations were continued for 8 or 16 min and were terminated by addition of 25 μl of 1.3M sucrose containing 4 mM of the P2X7 receptor antagonist Reactive Black 5 (Aldrich). Cellular accumulation of ethidium was determined by measuring fluorescence (excitation wavelength of 530 nm and emission wavelength of 620 nm) from below the plate with a Canberra Packard Fluorocount (14 Station Road, Pangbourne, Reading, Berkshire RG8 7AN, United Kingdom) or a FlexStation II 384 from Molecular Molecular Devices (660-665 Eskdale Road, Wokingham, Berkshire RG41 5TS, United Kingdom). Antagonist pIC₅₀ values for blocking ATP responses were determined using iterative curve fitting techniques.

### Fluorescent Imaging Plate Reader (FLIPR) Ca Assay

Studies were performed using NaCl assay buffer of the following composition for human P2X7: 137 mM NaCl; 20 mM HEPES [4-(2-hydroxyethyl)-1-piperazine-1-ethanesulfonic acid]; 5.37 mM KCl; 4.17 mM NaHCO₃; 1 mM CaCl₂; 0.5 mM MgSO₄; and 1 g/L of D-glucose (pH 7.4).

Human Embryonic Kidney (HEK) 293 cells, stably expressing human recombinant P2X7 receptors, were grown in poly-D-lysine pre-treated 384 well plates for 24-48 hours at room temperature (for a time sufficient for growth of a homogeneous layer of cells at the bottom of the wells). Alternatively, human osteosarcoma (U-2OS) cells (commercially available), transduced with modified Baculovirus (BacMam) vector to deliver the gene coding for human P2X7 receptor (i.e. transiently expressing human recombinant P2X7 receptors), were grown in substantially the same conditions as for the HEK293 cells except that the well plates were not pre-treated with poly-D-lysine. (The cloning of the human P2X7 receptor is described in U.S. Pat. No. 6,133,434, e.g. see Example 3 therein). The cells were washed three times with 80 μl of assay buffer, loaded for 1 h at 37° C. with 2 μM Fluo-4-AM [4-(6-acetoxymethoxy-2,7-difluoro-3-oxo-9-xanthenyl)-4′-methyl-2,2′-(ethylenedioxy)dianiline-N,N,N′,N′-tetraacetic acid tetrakis(acetoxymethyl)ester], a Ca²⁺-sensitive, cell-permeable, fluorescent dye (Tef Labs. Inc., 9415 Capitol View Drive, Austin, Tex. 78747, USA), washed three times again (3×80 μl), and left with 30 μl buffer, before the addition of 10 μl of the assay buffer containing the putative P2X7 receptor antagonist compound, the compound being added at 4× the final assay concentration chosen. The solution of the putative P2X7 receptor antagonist compound was created by (i) dissolving the compound in dimethyl sulfoxide (DMSO) to create a stock solution in DMSO at 200× the final assay concentration, and (ii) mixing 1 μl of the stock solution of the compound in DMSO with 50 μl of the assay buffer to create a solution at about 4× the final assay concentration.

The cells were then incubated at room temperature for 30 mins, before addition (online, by FLIPR384 or FLIPR3 instrument (Molecular Devices, 1311 Orleans Drive, Sunnyvale, Calif. 94089-1136, USA)) of 10 μl of the assay buffer containing benzoylbenzoyl-ATP (BzATP) such as to create a 60 μl μM final assay concentration of BzATP (BzATP was added at 5× this final concentration). The BzATP concentration was chosen to be close to the EC₈₀ for the receptor type. Incubations and reading were continued for 90 sec, and intracellular calcium increase was determined by measuring fluorescence (excitation wavelength of 488 nm and emission wavelength of 516 nm) from below the plate, with a FLIPR charged-coupled device (CCD) camera. Antagonist pIC₅₀ values for blocking BzATP responses were determined using iterative curve fitting techniques.

The compounds of Examples 1-3, 5-11, 13-18 and 20 were tested in the FLIPR Ca Assay (using HEK293 or U-2OS cells) for human P2X7 receptor antagonist activity and Examples 1-3, 5-11, 13-15, 18 and 20 were found to have pIC50 values of about 5.5 or more in the FLIPR Ca Assay. The compounds of Examples 1-20 were tested in the Ethidium Accumulation Assay for human P2X7 receptor antagonist activity and were found to have pIC50 values in the range of from about 6.1 to about 8.3 in the Ethidium Accumulation Assay. In particular, Examples 1-2 and 4-19 were found to have pIC50 values in the range of from about 6.7 to about 8.3 in the Ethidium Accumulation Assay.

The compounds of Examples 21 to 87 were tested in the FLIPR Ca Assay (using HEK293 or U-2OS cells) for human P2X7 receptor antagonist activity and Examples 21 to 35, 38 to 40, 42 to 64, and 66 to 85 were found to have pIC50 values of about 5.0 or more in the FLIPR Ca Assay. The compounds of Examples 21 to 87 were tested in the Ethidium Accumulation Assay for human P2X7 receptor antagonist activity and were found to have pIC50 values in the range of from about 6.2 to about 8.6 in the Ethidium Accumulation Assay. In particular, Examples 21 to 32, 35, 38, 39, 45 to 50, 53 to 58, 64, 66, and 73 to 85 were found to have pIC50 values in the range of from about 7.0 to about 8.6 in the Ethidium Accumulation Assay.

1-17. (canceled)

18. A compound of formula (I) or a pharmaceutically acceptable salt thereof: 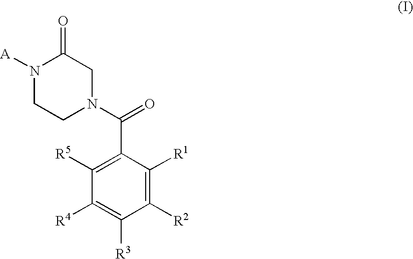
wherein:
A is C₁₋₆alkyl, C₃₋₆cycloalkyl, —CH₂—R⁶, —CHMe-R⁷, —CMe₂-R⁷, or optionally substituted aryl;
wherein, when A is optionally substituted aryl, said aryl group is optionally substituted with 1 to 3 substituents, which may be the same or different, selected from the group consisting of halogen, C₁₋₆alkyl, —CF₃, C₁₋₄alkoxy, C₁fluoroalkoxy, cyano, NR⁸R⁹, and pyridyl wherein the pyridyl is optionally substituted by one methyl;
and wherein:
R¹ is chlorine, fluorine, —CF₃, cyano or C₁₋₆alkyl;
R², R³ and R⁵ independently are hydrogen, fluorine, chlorine, —CF₃, cyano or C₁₋₆alkyl, such that at least one of R², R³ and R⁵ is other than hydrogen;
R⁴ is hydrogen; and
R⁶ and R⁷ independently are C₃₋₆cycloalkyl, C₁fluoroalkyl, —(CH₂)ₘ—O—C₁₋₃alkyl wherein m is 1 or 2, —(CH₂)ₙ—CN wherein n is 0 or 1, tetrahydrofuranyl, tetrahydro-2H-pyranyl, unsubstituted pyridyl, or optionally substituted phenyl;
wherein, in R⁶ and R⁷, independently, the phenyl is optionally substituted with 1 to 3 substituents which may be the same or different and which are selected from the group consisting of halogen, C₁₋₆alkyl, —CF₃, C₁₋₄alkoxy, C₁fluoroalkoxy, cyano, NR⁸R⁹, and pyridyl wherein the pyridyl is optionally substituted by one methyl;
and wherein:
R⁸ and R⁹ are taken together and are: —(CH₂)₂—X—(CH₂)₂—, —(CH₂)₂—X—(CH₂)₃—, —(CH₂)ₚ¹—, —C(O)—(CH₂)ₚ²—, or) —(CH₂)ₚ³—CH(R¹⁰)—(CH₂)ₚ⁴—;
X is O or S;
p¹ is 3, 4, 5 or 6;
p² is 2, 3, 4 or 5;
p³ is 1 or 2, and p⁴ is 1, 2 or 3, provided that p³+p⁴ is 2, 3 or 4; and
R¹⁰ is OH or C₁₋₃alkoxy.

19. The compound or salt as claimed in claim 18, wherein:
A is C₁₋₆alkyl or optionally substituted aryl; wherein said aryl group is optionally substituted with 1 to 3 substituents, which may be the same or different, selected from the group consisting of halogen, C₁₋₆alkyl, —CF₃ and cyano;
R¹ is chlorine, —CF₃, cyano or C₁₋₆alkyl;
R², R³ and R⁵ independently are hydrogen, fluorine, chlorine, —CF₃, cyano or C₁₋₆alkyl, such that at least one of R², R³ and R⁵ is other than hydrogen; and
R⁴ is hydrogen.

20. The compound or salt as claimed in claim 18, wherein:
A is phenyl optionally substituted by 1 to 3 substituents, which may be the same or different, and being halogen, C₁₋₃alkyl, C₁₋₃alkoxy, cyano, or NR⁸R⁹;
or A is unsubstituted naphthyl.

21. The compound or salt as claimed in claim 18, wherein A is phenyl substituted by 1 to 3 substituents, which may be the same or different, and being fluorine, chlorine, bromine, C₁₋₃alkyl, or NR⁸R⁹.

22. The compound or salt as claimed in claim 18, wherein A is phenyl substituted by 1 to 3 substituents, which may be the same or, different, and being fluorine, chlorine, bromine, methyl, or NR⁸R⁹; provided that one of the phenyl substituent(s) is NR⁸R⁹.

23. The compound or salt as claimed in claim 18, wherein A is substituted phenyl and has the following sub-formula (a): 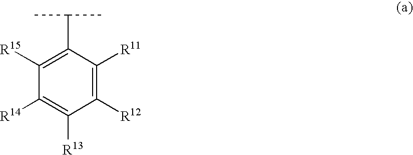
wherein:
R¹¹ is chlorine, C₁₋₃alkyl, C₁₋₃alkoxy, or cyano;
R¹² and R¹⁴ independently are hydrogen, halogen, C₁₋₃alkyl, C₁₋₃alkoxy, cyano, or NR⁸R⁹;
R¹³ is hydrogen or fluorine; and
R¹⁵ is hydrogen.

24. The compound or salt as claimed in claim 23, wherein:
R¹¹ is chlorine, methyl, or cyano;
one of R¹² and R¹⁴ is NR⁸R⁹, and the other of R¹² and R¹⁴ is hydrogen;
R¹³ is hydrogen or fluorine; and
R¹⁵ is hydrogen.

25. The compound or salt as claimed in claim 23, wherein:
R¹¹ is chlorine, methyl, or cyano;
R¹³ is fluorine; and
R¹², R¹⁴ and R¹⁵ are hydrogen.

26. The compound or salt as claimed in claim 18, wherein R⁸ and R⁹ taken together are —(CH₂)₂—O—(CH₂)₂—; —(CH₂)ₚ¹— wherein p¹ is 4 or 5; or —(CH₂)ₚ³—CH(R¹⁰)—(CH₂)₁₃⁴— wherein R¹⁰ is OH, p³ is 1 or 2, and p⁴ is 2 or 3, provided that p³+p⁴ is 3 or 4.

27. The compound or salt as claimed in claim 18, wherein R¹ is chlorine.

28. The compound or salt as claimed in claim 18, wherein:
R² is hydrogen, chlorine, —CF₃ or methyl;
R³ is hydrogen, fluorine or chlorine; and
R⁵ is hydrogen, fluorine, chlorine, or methyl;
such that at least one of R², R³ and R⁵ is other than hydrogen.

29. A compound which is:
4-{[2-Chloro-3-(trifluoromethyl)phenyl]carbonyl}-1-(2-methylphenyl)-2-piperazinone;
4-[(2,4-Dichlorophenyl)carbonyl]-1-(2-methylphenyl)-2-piperazinone;
4-[(2-Chloro-4-fluorophenyl)carbonyl]-1-(2-methylphenyl)-2-piperazinone;
4-[(2,3-Dichlorophenyl)carbonyl]-1-(2-methylphenyl)-2-piperazinone;
4-[(2-Chloro-4-fluorophenyl)carbonyl]-1-(2-chlorophenyl)-2-piperazinone;
1-(2-Dichlorophenyl)-4-[(2,3-dichlorophenyl)carbonyl]-2-piperazinone;
1-(4-Chlorophenyl)-4-[(2,3-dichlorophenyl)carbonyl]-2-piperazinone;
1-(2-Chlorophenyl)-4-[(2,4-dichlorophenyl)carbonyl]-2-piperazinone;
1-(2-Chlorophenyl)-4-{[2-chloro-3-(trifluoromethyl)phenyl]carbonyl}-2-piperazinone;
1-(2-Chloro-4-fluorophenyl)-4-{[2-chloro-3-(trifluoromethyl)phenyl]carbonyl}-2-piperazinone;
1-(2-Chloro-4-fluorophenyl)-4-[(2,3-dichlorophenyl)carbonyl]-2-piperazinone;
4-{[2-Chloro-3-(trifluoromethyl)phenyl]carbonyl}-1-(4-fluorophenyl)-2-piperazinone;
4-[(2,3-Dichlorophenyl)carbonyl]-1-(4-fluorophenyl)-2-piperazinone;
1-(3-Chlorophenyl)-4-[(2,3-dichlorophenyl)carbonyl]-2-piperazinone;
1-[(2-Chloro-4-fluorophenyl)methyl]-4-[(2,3-dichlorophenyl)carbonyl]-2-piperazinone;
4-[(2,3-Dichlorophenyl)carbonyl]-1-methyl-2-piperazinone;
4-[(2,3-Dichlorophenyl)carbonyl]-1-ethyl-2-piperazinone;
4-[(2,3-Dichlorophenyl)carbonyl]-1-(1-methylethyl)-2-piperazinone;
4-[(2,3-Dichlorophenyl)carbonyl]-1-(2,4-difluorophenyl)-2-piperazinone; or
4-[(2,4-dichlorophenyl)carbonyl]-1-(2,4-difluorophenyl)-2-piperazinone;
or a pharmaceutically acceptable salt thereof.

30. A pharmaceutical composition which comprises the compound or salt as defined in claim 18, and a pharmaceutically acceptable carrier or excipient.

31. A method of treating a human suffering from pain, which method comprises administering to said human an effective amount of the compound or salt as defined in claim 18.

32. A method of treating a human suffering from rheumatoid arthritis, which method comprises administering to said human an effective amount of the compound or salt as defined in claim 18.

33. A method of treating a human suffering from osteoarthritis, which method comprises administering to said human an effective amount of the compound or salt as defined in claim 18.

34. A method of treating a human suffering from Alzheimer's disease, which method comprises administering to said human an effective amount of the compound or salt as defined in claim 18.

35. A method of treating a human suffering from mild cognitive impairment associated with aging, which method comprises administering to said human an effective amount of the compound or salt as defined in claim 18.
